SEQUENCE LISTING (30 sequences)

SEQ ID NO: 1 - DNA, 52 bp, Homo sapiens
  variation (27)..(27): Nucleotide at position 27 is c or t
cataaggacactttgaggaagacccayttcctgcaggcaagcaggatgaagt

SEQ ID NO: 2 - DNA, 52 bp, Homo sapiens
cataaggacactttgaggaagacccatttcctgcaggcaagcaggatgaagt

SEQ ID NO: 3 - DNA, 52 bp, Homo sapiens
acttcatcctgcttgcctgcaggaaatgggtcttcctcaaagtgtccttatg

SEQ ID NO: 4 - DNA, 52 bp, Homo sapiens
cataaggacactttgaggaagacccacttcctgcaggcaagcaggatgaagt

SEQ ID NO: 5 - DNA, 52 bp, Homo sapiens
acttcatcctgcttgcctgcaggaagtgggtcttcctcaaagtgtccttatg

SEQ ID NO: 6 - DNA, 52 bp, Homo sapiens
  variation (27)..(27): Nucleotide at position 27 is a or g
ccttaatcatttgtaatagcaatttcrgaagcgtccaaagcttgtgcttaag

SEQ ID NO: 7 - DNA, 52 bp, Homo sapiens
  variation (27)..(27): Nucleotide at position 27 is a or g
tcttcttccttctttatgcatccttarctcaacattattacaagttatcttt

SEQ ID NO: 8 - DNA, 52 bp, Homo sapiens
  variation (27)..(27): Nucleotide at position 27 is a or g
acctgggtgactggaagtccgacattrcctgaagtaggcttggtgtatacct

SEQ ID NO: 9 - DNA, 50117 bp, Homo sapiens
ttagcaggcatcataaagttttacgtaccaagaaaatgttgctgttttctgaatactatgcatcaaaaaatgttaccactaatttttaaagctctgctaaggaatattggggcaccctcagatgcaccttttaattgatgtcatattttcctaatccatactttattcatgagaatttgagtcaccccagcattagcttggaatttccttatttcccatttgctttgcaggtgccttggagtcagatctggttttgaatactatcttcctgttatgtgatcttgggcagttacttaattttctagtcaataacccgtatctataaaatagagaaaataatcctacacaccggggcctgttgtggggcggggagaggggggagggatcgcatttggagatatactaatgtaaatgacaagttaattggtgcagcacaccaacatggctcatgtctacatatgtaacaaacctgcacgttgtgcacatgtgccctagaacttaaagtataataaaaagaaattttaaaaaatcctgtcaaataaggttatagtagagaataaggatgtgtaaagcatttagtcacgtaaatgcttaaaaaaatgtaatttttacttctttcactgcctcatttaattagttttatctttaataataccttggattcagggtaaagtttcagttatgtcccagtaatcatttattttaccctcgaatctgcaatttggatagaacatggtggggacagctcgtctctattccttgcagcattaacaggctggaggcaccacttctctggccagcaagttgggcctggttgttggctgagagcctcagttcctttctgcacaggttcctctttacataggcttctcaacagggctactagagcatcgtcaccatagcagctgtcttataacagagagtggtcggtctgagagacaaaaaatggaagctgccaaattgttctgggtctggaaactgtcagggcatcacttgtgccatattcagttggcctaagaattacagagcctgcctcgattcaaagggagaggatagagaggactgaaggaatcagtgctcatctttaatatgcagcaggacaggtttgggattttttttcccccttgagtctgtgaaggcattacttaagaacaaagtcaggcatgtataattgaactacagttacttgaaatataagcccagaaagtttcagataataaatacaactatttttctgctgttacccttgtacctaaagatgccatcctaatccccagatctccacaactatacctacatagtagaaggttaaaatgtatccctctttttctggtgcatccagcaaaagtaatatcatgaattatgagctctctgagagcaaggatcatatcagtcttgtttattgttgcagtgaacaagtacagttgcagatattcaggagtaattatctaaatggcagtaggcttataaaactgaattttcaccagccacaccctccccccaactccttatctgtaaaaagcttatttgagtggttacctgtcttcagtaaagattgcgcttgcatatttgctgtcattgcatattctgcttaattaagctctgttgatattgcagtttctgtgcatacttacatcttagatgcaatctgagggcctaggaaggccttttaaaaataaaacaattccgattgcagagaaagtgtaagtcaaggacagttaattcaaggggaacatagaaagctatttagattttagttgatggtgccagtcttcagcgtaaagtcaaaagtggagggaagtttagtaaggaaaaaatgttgggcttggaatacattgtttagtcttcaaagcactttactttttatgaaatatattttagacattcagcaaatattgaatacttactatatcaggcagtaaagatataaattcattcttaaaatgtgcaacatgttcaaactgaaaaaaatacattcttaaacaggaaactttttccttcatactttttaattaacaagacatataagagttgcattaatgggcgtgcttatgattgatcacccagcagcatcattagaaataatatattttattcatgtgcagaaatcttttggttgtcctggggaaccttgaacacagaaaagagcttttattgataaggtaattgaacacacttgacaattagcttaatatggtttaataccatttgtgggagaagatgaatcagccaggctctttacgtcaagaatatgaagtttctcttgagtcaaccaacttaagatgagctacggagactgcagtgaaaagttaaatatccaagtacaccagccaatttcacacagtggaaccatgctgtcctcgggcaccctgcacctcgcccaacagtcatcaactagatggaggctcctggctgcaaggaggatttgatgggaatgagtaaatgtgtcagcatagtccgtcccttctaatggaaaagcaacccaaagagcaaatcctattaatggctggatcagtatcatctacttgtcaaaaacattccatgaattatgagtcaaaattttatttatggtggcattacacacattaagagatgaggacttctgttagcataatttattagctggaaaagttgagaaggttctctggactcatttttataggtggaacctaagtgatctggataattgcccaccagcaaaattgctgggcatggtggacaaagaaaatgttccttctaatgattttttatgagctgagtagctattgttcccagctgagtgctcttttcctctttttattgttgctgagcaaaagaatttataaaaagctctttcttttgtattaaaaaccctgctcaattgaaatgcaagttcattaagtaatcttcatttctcttcctgccataataaccctttccctctctgttcgattcaacagtatctagcagcactgctccaaattttaagtctgaacagactatattacatagatgtagagaaatactcaatcttcagcattaagagggagcttaatttcacacgggtggaatatgatcactcaggctagatgttggccataaatttcaaattagtatctcaacttagcaggggggatcaacagtggcaaacttcaattatgacaggataaaaatcacatagagatattggttcaatatggacatctaaactataatgctaaaagccaataattagaataagttcattttaagaaaagcattaataatattagctaacgtttagtacctgtgccaaacattctacctatgttaccttgattttcatagccagcctaagaggtactattatgtatccccattttacaggttaagaaacaggctcagaggagtttaggatcttttccaagattacatagccagtaagtggtggcactaggaaccaaattcagactctgaatcgcatgctgtttatattatattgcactcattctaaatatgtgggaatcagaatgaaggggcttgtatgacttttggctcattttttgatgcatgtgacctgggattataaatgtgaaattaggtttacgaaaggatccagtgtcattgtgcatcatgggcaaggagtacctaatctctttaattcttccctggaagcttacgatgtccatccaagtgcacatagcaaaagttctgttgtaaagtttagcagagtgactttctttgactcagagtgatgacggaggaagctttgataagattttatctgaaatgttcatggacaagagctttcaaggagaacatccagagcaaggttctgaagacagctcatgaaggtgaagcagcagacctggcacaagaaatgaagagagagctcagtgtattaaagatgaaaacaagaaaaccgaatatattgaaaggagcagagaggcaatgaaaacaagacaactgaaatgaggtaacttgcagcaattgaaagggaatttcagtacttttatagaattcttaaaaattgtttcctgctgtttattttcaattttgaacagggttatttgtccatgccatactttttttgccaaattccaaaattgtgtatagttctatagttgtctggtggagtcaatggaactttagttaccagtctaagaatgtgtctttgagattgtccagttaattctctatttccagtagctgtaataaatggtgaaaaggtttctgactcctggagaaagtttctaactccttatgactaatattcataacagacttgtgagttccttgaacatggatacacctatatgcaagagtgtattccaaagctaactcagtgatctttccatttatctattcttggattagtggtgcctttgctctttccttctgtaaatgtgaatagttaagagttgactgcagaagtgtttacactttggcttccatgcctctggaatgtttgtgctttggtggtgagatgtgagactatatttgtatagtctgcatctctcaggctgccccagaatgttgtacagtgcagtgctgaagaaagcagcaggtacacacagaaatgcagcctttcctggttaaccctgcttggatctgagttacactttgtttcctgacttcttgggacttaggtaatcagtttgccttctactctatctcattttgtactcgcttacatactacattcttgtttgggctttcgtttcttcttgtaagcagagattttttaaaatccaatatgtgaaaatacggatgcactacaattaaataaataaaatgctgttgtgtttgttttgctttaaaattgtaaaggataaacaataagatagttttatctatgtggttttcccgatgcagttaaaataaaacctaatctgctaaaattgaatttacactcaatttttctgttgtataattcttgaagaacacctcatgtgttgtacaacttcatctacattgtgaactcttttatttattgaaagtctatcttagtctttcactttttctacacttccctttacttcctcatgagatgctcagcccagttgtccctcaggacacagtatcgattggttcacttttaaaagcaactgttagaaacccaaatcatagacatgtgaattttctcagaagccattattgaaacttggatttttacctctcataccaaaataaagaagggaaaaaatagaaatcagcccaacaatacttttaaattgatttttatatttatgttgtaatgcaaataccaaagaaaagtgcctagttccttggtttatttacttatatcttgcctctgttatcttcatatcaatttttggccttagcacttttgactgcataaagaactctaaacataggcaatggtaatagaaatgctgacacatctgaactgtcaggcccagaatatattattgaatatgactgcacaaatgcctggaacctatacttagcgtaaagaataaggctgctgttttcaacttccaacaggaggagagtacagacatatgtgccctctgtttcccctctctggctctttcattgtctttcccctatgtgtgcatgcaggcaggcacacatacaaacacatcctgccccatgaagtggtcacatcttaatatctactaaggccagttagacattctgtgggccgagtgtatgttgcagttgcttaactggcctaaaggtctcatgatatcatgggcactatcgagtgatgtttccaaatctgtagaaggaggaggactggtagtgttgggcttgcataacataaagggcctgtctaccataaaacaggagcttggatttgtttttttcaggagatgaatatgtttttcacagcagtctagtgaattaagaacaatacacagaatcccacagcgagcgtgttctatttgaatctgttgatgggatttatgtatggaacaacaattttctgtatagacataattgaatacatattaccatgatctgaaaccatgcaagaatatgtatggtgtgtgggaaacctgaagacagatacaactgatgaaattattagtttacattttttattcaaaatatttgtgaagcagctattccctacatatgctctatgtagaaatgcagagacaagtagatgattataatgtgtcaccatctcttttttggactgattttttttttttaaagacggagttttgctcttgttgaccaggctggagggcaatggcacagtcttggctcaccgcaaccaccgcctcctgggttcaagcgattctcctgcctcagcctctcgagtagctgggattacatggactgaatttttaatagagcaggaatgccaaaagggaagtccctcagatctggtgtgtggctcaataatgcgctcagttccatgcagcttcttcccttagggctctgagtaaagtctctggtgccagaaggtatgagggtctcttgtatttaataatatcaagggagtacagtgttttccctcaaagcctggccagcagttactcctggagaaagaagaacaatctggttgacaaatgtgtcacccatgtaaactcaccagaaatatctagagccatgaacacctagtggggtcagcaataggtcaggcagtaattgctcagtgcctcgttcgctagcagcagtgggaactgctgtagtgtaaattggttttaggggccaaattgatcaaccaaaagaggcttagagatttttggtgtttgagttactgacgagggttccaactttctggatcacgtgatgaaatgcatggcagttgactctgctatcctctaggttttgtgtctcccttccaacccccatcagcaaccgtttcttgttcttgaggctgcttagcctaagaacttttggtctttgctgtctgggagttacaaatccacaatggcagtgaaagcttttctttctccctcatcctttgtcctttcctacttttggcctttttttgcttttgcaccagttacacttgtgtccatctgctagacacccacagcatcagggtgcttgggcgtggttcctgctgggcagttgaggagctctgggtccacgacccagctctccaagccgcctcaccttgctttccccttctgaaataccactcacctacctgcccacagagggagacagtgagactaacgagaagggcatagggagtaatgagaagaaatgcacagcgtgttctggactcctcgaagagaagtgtttgttcaaggcattattaatatttttgcaaggcacagtaacattgattttttcatccaagaaccatttcgcatttgcacagagtagtcactagagtgttgaaccattttcttttatacatggaggaagtggctcatcctcggcctaagagcaccaaatttgagttggcattttttatttccagttccaggcatgaatctttgctccattttgcaggtagctggaaaatcttctgtttgtttaaatccacgttgggtaatactgcatcattttaagttttctatctggagcccggtacaggaattcaccaatgccaagcacggactctgtgcatagatgccttgtataggctccaagctgcaagcctcccacactgatggatttaaatttttattttacatcccagccaatcaggtgtctgactataacttgaagccaattagtatttttcacagagccaggtgcaaagctgccaggtaacatctgcaagtgacttgcattgctctaatgggatctgcatatgttcagctctgtatgaagatgtttttcacttgacatgaagtcatctttggtcttgttctccctcgactccattaagcagacacacctacgtgtgtggccaatttgatccactttcaaaggaggatggtgcctagtgtttcttttcatgatctattatttatgctgatgcctcttggaaaactgtatctacccatccaaagtgctttgagaggggaggggaaggggtgtggcaacacatctctcagttactatcaaggcattttctgataatacgtggcccaacctaaattatccaataaagatttacttgaggactttgtaaatttcttttaaaaaatatatctgatggaggcaatttttttctcaaagaacatacctgagttcctttaactttgaggaacaaagacgtgggttctagtctcctagtttgcagctgatgagctatgtggctctaggcaggacagcctttgctgagccacttgcataggtgctcactgatggccataggacctgtagaaggagcacacctttccgagtcacactgacctcagtttgaatccctgaccctctctgttggcacagtcatcttgtggaagttttgtgacctctcttttggtttcctgacccatgaaatgggtataataatacatacctcagagggttatttggagagtttaatgctttcatatagacatgcctgcttagtgcctgtacatgatagcttttcaaaaactgctcatcttcacagcctgtcttttttactccactgcttttattttatttcaagtactatacaaatattccaagtggatttttaaaagaatggataactaccagtatttccaacatcctaacctcaactatattctttttttctggggtctcttctagtacatattcacataaagccttaggaattgatcttataggaaatgcctgtaggcaaatatgctcatctctagactggaaattgaaccaaagtattttacaacgacactgtggctaaataagaaaacgtgtttaacagctgaaaacgtcatccaagagcatttagaggaaaaaagaaacagtcctttggccaggattatggcaaagcaaggtatgataatattaacggctgttaattactaagtgcctgatatttgtgccaaacattctttatattatttctttcatccttatagcacccacttagctatgcatctcttttcaaataatgttttcaaatgcataacaaaaaaaaatttcacaggaaaccaattagtttaaaacaacattttttataaaattgaaaaacatttgtgatatagtaacgtatgtgcctcattattaatgcattaaatttcaagatttatttcacaaagaaggggttcaagagcataagtacttttcatagaaccacatggtcaggaaggcttctgctcccaggtcttctacctgcatagtctgtcctcttacccccttatcctgctgcctgaagacacttagggagcttaagactcttagcctaagttcctgaaaacttctctcctggcttaaagtgaatgctgggtaatttatttagtctgctcggggtagtcattcattccaacatgattgtagtccaaggacctcccttcctttcagaacatttgtcacttgtcatgttgcatttaggacgccagaggaggcatccagtgaaatgaacaggaggcacttttgtgacaaatagacttcggaagccaaagctggaactggaaaagaccttgcagatcttcaatcctccttaaggtgcagcctgatggatttccagatccgtgacattttttaaacacttgatcacagcaaaaagaataaacactgatttaaaaggaagacgccgggcctaggtgtaagagagaatgttgccatttttatgtcatcttcacagggaaatagcagatttcatgaactcagactttggagaaaggctaatgtggatttaaattccaggtcatatacttacttgtgtggctttggacaatttactgtaaaatggaggtatatcacctacttcacaggattactgtgagaatgaaataaaagtgatgcgtgtaacacattgacacttggctggcgctccgtaaatgtgtgcacaatacaattcctttcactaaaccagtgatcctctgctttgcctgtttcttagaatcccctgtgtaccttttacaagttctaatgcccagacagcaccccggaccatcagactttttggggtgggagccacgcaacagtatttctccatatccagccaacgttgagcatccctggactagatgcaggctgagtgaggcccaggcacagagggcagcctgtctgcaagggctctgcaggaacacagctcaggacggcagcaggggtcagggcctggggcagtgtggacctctcagctcactccaagagctgcaggcaggaccatccccagggcgtaactgagggcaaatcactctgagggcttttcttctgccacagcacttttatacaagcgaaacagaaaaaaagggcagcctggagaaagattatgatcaaagcaggcagcttaaagagtgatggtaaggaaaggggaaaggagacagccaaacggggtggtgagaaccagcctgcacccgcacatctgactgtgaaggctctctgactttcagtgcaactgtccaaagtgatcgtctctttcacccgctcagaagtcaaggactgggagactcattattttgaatagagtttccccgaatcattattgggaagttcaagtgtggtcccaggatcagcagtgcagcatcatcggagagctggtcagaaatgcagaatctcagaccctgccacagacctgctgagtcagaatctcgagtggtggagcctaggaatacgcattttaacaaattcacaagtgagcatgattgagaagcactgctgtgtatagtgaaaatagagcaaactttaggggcaaacagcacttttgagtttgagccctggctctttcacttactgtgtgatctttgaaaggttgcttaactcctctgaactcgtgtttcctcatttgtaaattggggatacaaagatttaccttaataatggagtccaaggaaattgtgaatgaaatagaggggggtctttggacttggattggaaaaaaaatcatttttgtttttcacaaatttctaactaaaatttagcattttcttttattatgaatgtcagcaacatttgttagcagcctgtgactttgtcaccagaacaagccacacatatttctttttctatttctttcttttttttttttgagacagagtctcgctctgtcgcccaggctggagtgcagcggcacaatttcggctcactgcaagctctgcctcccgggttcacgccattctcctgcctcagcctcccgagtagctgggactacaggtgcccgccaccacgcctggctaattttttgtatttttagtagagacggggtttcactgtgttagccaggatggtctcgatctcctgaccttgtgattcgcccacctcagcctcccaaagtgctgggattacaggcgtgagccactgcgcccctatattacattacagttgttacagataactccaaatatcatttgtattcatccagttcaaaattacagttgcttttaaacttgccgctagatatttaatgcattagtaatgaggcacatacaccatgatatcacaaatgttttttaattttataaaaactgcattttgaattaattggtttcctttataattttttttatacgcagttgaaaacattatttgacgagaggtccatagactcatggcacaaaacactttcaaaagccctccctgcctggaaagttgttgtgatgatttagtgatggaatcaaatgtgtccagcactttttagtgattcaaataacagtgccctgattgtcctccccaacattgtcacgtcttgctttgtgctaagcctgggccaaagattctctagactttcctgggggtgtgtgagaatacacactgtctggtcaactttccctgcaggcatttctctggctaatcctggttttatggaatgagagctgttgtgtgttgagtgcctgctctttgccagattggtgaagtatgctgccttgaatcacatggccagttaagtgggaaagcaggtgggtggtctccatgctgccatgtatattcctgggtggagctaagtccagatgttggcggacagactaaggactcctgcatcctcctagttcaagaagatgacagaagggctatcagtgaaatcactaagcacctacaatctcttagttcttgtacattcaataagcattaattaagcacttactatgtgcctggccctatgctagtcactgggatacgcagctggataggccgtgagccttggctctaagaagactctatgggccctgggagcagagaagggtatcccctaaaccagtggggcagaggtggggactgaaacaaggttcccctgagcaggtggttaatttgtcaaattggctgggcactgtaatgccccggtgtttggtcaaacattattctggatgtttctgtggggtgtttttggatgagattaacattctaatgggaggactttaagtaaagcagattgcccgccataatgtgggtgggccttatccagtcagtcaaagactttcataggacaaatactgacctcctccaagcaagaaggaattctgccagcagacagccttccgacttcagctgcagcgtgacttcttccctgggtctccagcctggtgacccacctggcagattttcaactttccagcctctataacctcctgagcaaatctcttaaaatggatctccccttctatctgcacctcccattgattctgtttctctggagtgccctaatacaaagtccaactggctatattcatttgtgattatgctcctatgtgctttactttttatttgattatgtctcattacacagttcctgtcctggggttctgggcccctgagggattttcattttattacatagaccccttgtgacaatggtgtatctgggctaatgtaaccagtgctgtttcactgggcacaaactatttggggtgcactgggaagcatacatgtaggagggaaaaggaaagacatttattagtgcccactgagtgccaggccctgtgcattttacactaatttcaacctctaatactcggtatacaacaaggtacacagaattattcccattgcacagacaaagaaactgaggtctggaaagggtaagtgacttgtctgagcacaccctgctaataagaagaatcaaagatggcacccaaatctcaggatctgcctgacacccaagacacccgaggccaggctctttaaaacataaaatgctgtttcacacaaaaggtgccaggccaagggaagaggagccccttctcaggacagttgtggttccatagtccattaaaccttcccaccaccaaggggccctgcagccaccccaccccctcccatgagagatcctctgcaccttgcttgaagctcatcaggaaggtcaccaaattgtaaaacaaaggaatccttcagtctcaggaggatctttgaaacaccgcacctcaatccttggccccagagcctaggatccacccagggctccagggcctcaggcatttggcccatcaagtgctgcttgactcctctgtatctcaccccattgtgaccctggcattttgctcagctgatgaggatatcatgtcgggcagcattgcagctcttgtgcagcccgggagattgcatctattgttgtttccttatctgtgtgctctttcattctatcagacaaggaaattaaattggtctggcaccatttgctctctagaaagctactttggctgctaaatccaccaaagggggtggatttatttgggtgtttctttcctaagtgagtgaacaggccttttcagagccgcccacaattaactgaaaggcccgagctgctgtgccaggcccacctgttgtggggatggaagccaaatggggcagatgggatcgaagcggggccagaaaaatagcctcttaatttagcaccagagtcctctcccactctgctccagcccccctacaactggaaaaatgtggaaggcacaaatatgagtcagagaaaaccatccaggaacctgggtgttaaaaccccacctccagactgcagcagggaacgttaggaaaaaagggctgaaaaccgaacatcaatattggttttgctcctgatgcctggaagggtctcccttgccttgtcctcttcatgaatgagcattcacctcttcatcctccaaagggggccctcgcagttttccgcagcctccgcgtccccatgtccccaggcagagagcccactggcccttttgcatctccggcatgttgaaatgacctgttggcatacttacgggctgatattattatggttttttttttttttttttgatttgccccaaaaaggagttgaggtggattacaaacttataactgaactttaaaataaatcactcacttgaaagttggtggcaactttcaagatcagggcaaaaggaaataagaaaaatttatgtaaaatatttactctggggtcttcttggtgggccatggctttggccctcagctttccagcagccaacccgaagctggtgcgtatcagttccttgattcacgctgttcacagctggtcaggtaaggcacggcttttccagttctgaggcctaagaggaactttgctcatagtttctcataaaaaggaccctgtgaggggtcatgaacagtgtccttaacatgtcatggacattgtccttaggtagacatatgtttcatgggactgtgtatcatataacatcctttctgtccgcctatggcaccacatcaccatgcccttcggggaaggcagttctacagggtggccaatacaatgggacttggctgtagtttcctgttagcctgcctagattcacaataggctttccggaatctagaggattcaggatgtagttctcgcttgcctcagtggagacttgaatgagtttatgcagtagtcatgaatgaatattcattcttaccatcgctgctgagcatctgagcccgctccttggctttgtggaattccctgagctaaggcagatcttgcctcccattttagagactgaaagtggcagctacttgcttttctagcctcctttgcagctaacaggtggccagatggcccagtcaccaccaaacagacacatcgccccaagttataaccatgacaagggtgctggtagtggctgcagaaggataatctttttagggcagcacgtgcagcacactgagtgggggcatccagtgactggagctagcagtatcagcaatggagactgtgacctcctcacttgacccaagctactgtccaggccgtaggagcctgaacttattccttctgccctcctgctgaatgtgtgatcttctcgatgtccttgtaatgagtgtatattctgcttaaatcagcctgagttggcttggttgcaagaaaaaacctgcttgatgcaagcagctgttcttctccgctccccgcccacccacatgcaaggcataggaaagtcaaactgctgtgaaagccaaggatgcgtacacgcataaggacacaagccaggtctgctcgcagagttcctgtgatagatagacggacagaaaacttgagggccctgcctgcagcacaggcctgggggcagaaagactgtcccagagcttgctagcctggaatccacagtctagatttatactgtccaatatggtagtcagtagtcacatgttactgtttacatatgaatttaaattagttaaaatttagtacgaccactatggagaatgctttggaggttcctcaaaaaaataaaaattaggctaccataggatccagcaatcccactgctgggtatatacccacaagaaaggagatcagtatatagaagagatatctgcactcctatgtttgttgcagcagtgtttacaatggctaagatttgggagcaacttaagcatccatcaacagatgaatggaaaaagaaaatatggtacataggccaggcatggtggctcacgcctgtaatcccagcactttgggaggccaaggcaggtggatcacctgaggtaaggagttcaagaccagcctggacaacatggtgaaactccatctctactaaaaatacaaaaattagctgggcatggtggtggttgcctgtaatcccagctactcgggaggctgaggcatgagaatcgcttgagcccaggaagcggagtttgcagtgagctgagatcacgccactgcactccagcctggggaatagagcaagattttgtctccagaaaaaaagaaagaaagaaagaaaatatggtacttatattaatacataatggagtactaatcagccataaaaaagaatgagatcctgtcatttgcaacaacacagatggaactggagaacattacgttaagtgaaataagataggcacagaaagacaaacatctcatgttctcacttatttgtaggatttaaaaatcgaaacaattgaactcatggacatagacatagagactagaaggacagttaccagaggctgggaagggaagtcgggggctggatagggggaggtgggtattaaaaaatagaaagaatgaataataactattattttatagcacaacagggtgactatagtcaataataacttaattgtacattttaagataactccaggagcgtaattggattgtttgtaactcaaaggataaatgcttgaggggatggataccccattctccatgatgtgcttatttcacattgcatgcctgtatccaaacatctcatgtaccccataaatatgtacacctactatgtacccacaaaaattaaaatataatgaaacaataaaaacattaaattaaattagttaaaattaaatagaattaaagatctgtttccacaggcacatcaaccacttacggagtgctcagcagccacatgcacctacagactaccattatgtacaagacacatagagaacatgtccagcatcgcacaaggttccattgcacggtgatggcctagaaccttgacattccaggctgagtggcaggatttctgcctttttgagtcacacctattgcttctggtggctgacgaggctgggagttcccaagcaatgaggcagcagttccgccctcctccatcctggtggtctgaaatccacaccagctaccaggaccacagagcactgggccaaggctctgggaaccctcttttccctgctgcatcttcgtctatgtcccttttgctacttcttgttggctgcttcagggggagacaaaagtaaagacaaccttgggtcaagttctcaccatcattctcattatcctggaacctggaccaggctctgcagcctgggctggtgcagaggccatctcaggtatgcaaatgagtgtgttatgcaaatgagtgcttcctacaaataaatgctttatgcaaatgagtttttgcattctctatctctcacactgggctgagcagggactttgtacttaccccaggacctggcacagggcttgacacatcaaagtactcaggaaacattcactgggtacacgtggatgtaacacagatcccgagggacctgacgcacgacaataatttccatgcagagtgtcttcctttcagtccaaggcaaggggggtgacacagtaataatgatgataatgagagtgatgatatgtatatgaaaaaatgaaagtatagagtatgagtttattataaaagtgtaaagaaattggttacatgtgtaaggaaatgtggagttatgggcatatttttcttttcaaattccttcaacattgttattacccatatcagagtagttaagaatatgctcagacagacatggtccccctttgtgaaatgggagctgttacctcatttggctatgtaaggactaaatgagataatgcatgaaaaaattcagcactgcctttggcatataataagtgctaagtacaagggagcaattattgttatttttgcctagaaattcgtgtgataacaatagagcggagcgggactcttggcgtccctgtcattttgtggctgttgcttccctccccactggagtggatggaaagctctgggcctgggtgtgatttctgtcctgacagcccagccctctgcccacagggcaactgggccaagtccccactgcgtgctcccagcaagagggcactgccaaacttctcatacctttttatgcttctcggtctaagccatgggggtttggagggatttttttttttttttaatttgagatctgtcatccttgcaaatgagtttgccctttgcagcaaatccacacaaaacagggacaatctccagttttctttctgttgccttttctcctcccttctgttcgagtcctgcagctactaatgagggcctctgtcagctccctgggagggcaggaagagctgccttcgccctcctccctctccctctgtctctctctctcgcaaacacacacacacagactcaggagagctgcccttctcatctggtcctccttttcctcctttcccccagcttctgccctttatcttgctgttttcacacttccttcctccatctctcctgtgactctttttctctgaatctcatcttctcttctatttcctctcgcataataagtgtgatataagtatttgctatgattatgatgtctcccccatctctttctctctttccctctctctcttcctgtgtctatctttgtaatctttctgtctctcttggtaactggctatttctgtctctcactggctctgtctatctccctctctgtatgtatttctctgtcgtcatctttctgtgtgcatctatttctgtccctctcctctgcctcgtctgtggctctctggctggctcttcatgtcccttaaatcttctaagcccctctgagggctgtggtctgagcacaggaagtaataaggaggagtatgccccaccctgaccccagccaccccaaatcctcaccacagtgcctgagaagtggatccgctccctcttggcccaagataaaaatgcatctctcttcttacagcctcttcctacagagaaaatactgttcatggggtcctgcctggaaaaacttctggttgaggaagtcattttgcgatgcttgcaaaacaaactttcagaaaccaaatttcttgaaacaaaatctttgtggctgtttaactgatttagtctactggtaggcacacagaataactaagactaacacgtttatagttaaagcattgcactttatcttcaccctttcataatctactttttgctacttcagcccactcttcttgtttctcagcataacctttttgtagtaggagaggtgccattattattatctccatttttaaaagtgggaaccgatactcataaagatgcgatgatttgctccctgtcacggagttattttcagtgccgcagggcgcagaacgcttattccctagggaccatgagcccctctcgcgcccctccatgcccctgtcagctctagggctctgtgatttccaccagaaatcccaaacatctcccaccaaacctgcccaagggagactctttgtggctttttcgcacatcgtgtcctttgtaagtcatcgttcccgctgcacacgcgcccaccatctccaggctgaacggctcagagatttaatcacgttctcggactcaggccatggcagtgaatcttggagccacgcggccggagctgcagcagaaaccctggcaacggaaggagctctctgagggtttggtggaataaattactcaaacacttgcatttctacaaaagtagtgacacttaattcctgctatgatattaggataaacaaaatgtcatctgccgcattgtccttgcccatccaaatcttatcttctttttccgtgtaagtaattgggggaaaaaaattacagaaaccttctggagtattatcgtattaggatccgattttaaccagaatgctgaaaatttcatactgaaggcaagtgcatagttgccgtaaatgcaggcgaacttcaaggtgaaggaactcccctgtacaatcgcgaatgcaaatgtttaaagccactcaacaattttccccttgcagctgcccgttttatctcttgcaaaactagtaatgatacaagcagttgattatggtttacaatagcaataaacctctcagaattggttgtttccaaaataaatagccattctgttaaatgttaaataacaagtcataaactgctgaaaacaaccaatttcttgtggattattgcctgaataaatttcagcaggaacggcagatcattaagctgtgagcgtcacagatcaaaaccgttggattaaaaaaaaatcaatcaattaacttaaatccatccaaaggaattcccattttacaaaccaagtcacaccacacatttttacatcttcttttgagcttttcctttgctggtcatggttagcttgattctggccaaaatcttttcattctttttcagagattaaagcatacctaacctgggtggttaaattggctcattctgagagcatgcagctttcctcagctgcattttctacaagtctccatctacagtgttccaattgataaaacacagacctgtccactaagttaaacatttgagtcaaggctcagtggcaaatatatattcaaactgcagctctaatagaggctgttctggtaagagaagccaacttcatcgggatgattgacagaagattcctcgccatagcaaccagggttatctctctggaataacaacattaacctccttcataaccagtttttcatccctgaatataaaaggcagctgcatgcatcttgtatgatcctttttttttatttttgaatgaacagttagcacttctatataaatgggacaagatgcagcaaaatcaaattaattttgaattcctcttgaaaaaaagcatggcaatggatcttgtaggcagaacagttagagcaggaagggttcctgtctcttctgagcatagggcagtagcagtgagatttctagtcatctactgcctctgaaatagcctcctgcattgatttgctttctgctgctgtggagggtgggaaggggcaaataatttgtttcttgttttgttcctgtcttattctttgtcctcacaactcccaacgcatgcacacacacacacacacacacacacacacacacacatccacacacatcttagaggctctcaggtgcaaggcaatgaatggtctatgggaaaatggtgggaaaatgcatattctttctgaaaaaaaatgtatattccatcgctttttttgaaagaaattgctaaatggaccccaaaccatctcctctgacttatatctaatgttttgtgaaagccgtggacattggtgccttgaaatgtcactgaagcctctcaccagcccctcacagaggtgctgtgccatgctctggacctgacttctcatgccagtctctgcaagtagcaagcatggcactcaagccacagggaataaaaactgtgacaaggaaagacagactgtccacctggttggaccactgccaagtcatgtccttccctgtgtcattccatctaggatatttttcccattggcaccaacttcattgcaattgccaccttgaaaagtggagccctcttggttgaggcttctgtgtcactcaaggtcacaatcttgttgctcaaggccctggttgtaacctcaaagaacgtcagtcctctgctgaggaaagaggctgagcagggaaaaggtgccacctgggcccaagcaaggggaacctgggaacatggcatttacaaagtgcaggacagcgtggggcctcagaggggaaagtggtcattcagcagaaacactcgggtgaatctctggcctgatctctatgtaagcagacaaactagccaaaaggcatgactggtggatttcaaaagatgttctgtgaagaaatgttatttctgagttggaagtaggaggaactctttgagatccccactgtattacacaaatgttgacagcctcctcagattccttcaaagccaccttacttccaaagctgctgcctgcccctcacacccctcaccccaccaacagcatcactgtctttgccaagccctggaactgcctggaatccagttggggccctgagtctgtgtgaccctgttaccagaaaggggtctcaattcagaccccaagagagggttcttggatcttgcacaagaaagaatttggggtgagttcatggagtaaagtgaaagcaagtttattagagaagtaaagaaacaaaagaatggctactccataggcagagcagtggcatgggctgcttgactgagtacacttatgtttatttcttgatgatatgctgaacaaggggtgggttattcacgaattttccaggaaaggggtgggcaattcctggaactgagggttcctccactttttagaccatatagggtaacttcccagtgttgccatggcacttgtaaactgtcatggcgctgatgggagtgtcttttagcacacaaatatcacaattaatatataatgagcagtgaggatgaccagaggtcacttccatcactatcttgggtttggtgggttttggctggcttctttactgcgtcctgttttaccagcaggggctttttgacttgtatcctttcatactagtcctgccaacctcctatctcatcctgtgactaagaatgcctgatctcctgggaatgcagcccagcaggtctcatcctcattttacccaactcctattcaagatggagtcactctggtttgaatacttctgataaccccatccccactgctgagcatcctcattcctggtcacttttccactccaggatttggataaaataccacagccatgggcatgggacctggcctcctgagcagctcctgaggagccagatgatacccagaaatgcaaggtgttacctccatggtgtgtgaacctgaaccaaggtaactggaggtggagagtatgtcctccctccaccctgaggtgtgattcaacctggctaatcttctgcagaagtcccatgagctgagcggacacactcagggcagtttgtgtctctagccactcaccaagaagcagcatctagagcagcaccattgcatcaccttgatcctcatctttcctgcctttcctcctctttctttcctcacctctccaaggttacacctctccaataaagcagccgcactgtaatccttgcctcagcctctgtgtggaggacggggctaggaaaactagctacatgagtcatactcagagctcctccctgtggcaagcattttaggtaaaggagtaggtggtgaaaatcatggcctttctctacgaatcagtccaaagactctccccttcatttgctaaatgacccctgatgttacagccacctctggaagctgcaagtttcccaaaagctgaggttgaggaagggtcggttggttaattgtttttaagaagcagtgtaatggaccttattacttaattgacaaagggcttgactggtggaaaatgaagcctccttgcctccaagatgttgcaattagttttctttaaaacagaaaagaaatgttcctgaataatcaaaagaacaatagcaatagtagcaacaccccaagttacaaattgcctgggctattggcctcctttaaacacacacatagggggcattgctttttcctgtccagaggaaagaaagctgatgctcttcccctctcactcaatgccctcctcacaaagtcttgctgaccctgcctcgtgtaaatatttcattatttacgggtgtgttggtttattcacgcacctgcccagccatcctgtgatctctctgtttgtgtggagcctgtgtgtgactagttggttgcagctggtgctaatgaggccaaagcctgggttcaatccccaagggggccagttaacttcacctgctccgtggctagaggctgcactcaacagctctgaatggagcttgtgttgtgtgtactgggcgacaggcacaggggtagagggaagggaggagatggttcagcatgtggtccccatgtctgctggagaaacagccacagcccttggcaccttccacccctgcaaaggtcagcactgtgtttcttgttttttttccccctaagattacttcttttatcctaaggttagaggacgtgggcatgtatgttttaaaatttcccctttttatttaattggcaagtattatgtcttatgagttaaagagtaaggcacatttttttggcctcaattcctttgggcatccattattcatttactagaacacacagcctactggagaggtgcatctgtggacatcagaaaaaaaaaaactaggaggagttacagattgtggcataaggtttggtgaaaatattcaagaaaaaaattattcactcaacaaatacttattgactgcctattatgtctcaggcactacactaaagatacaatagtgaacaaaatagattttaaaatttctgctctatggagcttaaattccagagggagaaggtggtgcaatagatagtaaataagaaaaaggtagtaaaaaatatgtattttacataaccataattttgaagaagaatagagcaggaaatggacagaagtcactaggaagggtactccaactataataggatggttaaggtagacctcagtgaaaaatgatgttagggcaaggacctgaagtaagggagagtgagctgcgtagctagcagagggaagagcaaaggtgagaagggcaaggagggagcatgcatagtgcctggtatgtttgaggaacagccgggcgggggtgcagtgtggctgaggcagagggagtgagggagaggcgatgttcctgaactgaactggggtgtgctcacctgctgcaataagatcaaacatccacacagaggtttgcagagggagaaaggagggcatttatttgcagggcaccaggcaatgttgattgggcagctcatgcttaagacctgaacccttgcaaaggtttgcaagcgagggtttttaaaggcaagggaaattttcaggaaaacaagttaatacaggcaaatttaaatcaaggcatggaggttatacattggtttggcctaagaaggtgggacaccttgaagcggggcctttacacgtcataggtgggttcacagattttctgatttgcaattggttaaggaaaagaagctttgtttaaaaatgtggggttagcagaaaagaacgttagctctggctcacaggtgtgacttcttccaggcccatcaggaagaaagaacgaagagcagtggtcggagttcagtccccggttcccccttatccaaggtctgtgtgccagcagatacatttggtgggggtccaggttttgaaaacaactcagggacatacgttaagaattaatctttagtttctatagggaaccaaacatcccatgactctaatttccttggctattgttttaggctactattaccttcttgctgatcaaattgctcatttacttctcagggccagctaagtgcctgcaatctgaaggaacttaagatttttcatttccatgcttgtggtagtgaggggggcacaggcccctaagaggggtccctgctccatctcaggatgggagatgcagccaagaaaaaagggaggaaggagaatcaggtggcgtctacaggcccttgcaaaagctttggcttctttctcaaaaagttagaaaaggcttcctcaaagatgttgcttctgagtcgggtcccaggagctgattagtgttaactcattcatctgcactccggctgaagcaggtgttgccagactgaccgtgtaatttggtagttaacatgtgtgtaccaggctgtgttttcagacttgaagacagagtggagttagcatctgctcctacccctccctcacccaccccatccccacgcactcatacacactaccggcctgggagtcatatgaagcttatatggtcccaccatgggggcccacacaaggaccgggattagtttgcccaccagccaggcaggtgggggctcagaacagataaagatttgtattatttcttgagccacagttatagatgtcagctcatgcttgctaaagaaacaaatgaaaattaactggcaaggcaaccccagggcaactgaatgagggagaaagcaaacactccccgggttccaagcaggaaggagccacatgggggctactttgggggcatggaggaaaaggtaattatagaacactttgaggagcagataagatttgagtgggtcttgaagcacaggtgagtgtgaatcttcaaatgggagggagagcatgacaagtcattgttcctttgtgaggggcggtaggaatttttggaaatgttcaggagtcaggaagaggaccatctaggctagaatggagtggctgtgtaggaaaagggctggcagagagtgaggcagattgtggaagtcccggaatgccagctggaaaagtttggacttttgctgagggtgattctctgctttggatggcaggactgtagcagacagaaccatgtctgttcccatatgcatataagtatgtgtgtggtagcctcgccagatataaaatgcaagggaaactctgttaagtttggatttcagatcaacaacaaataagttttcagtataagaatgtcctatggggccaggcacggtggttcacgcctgtaatcccagcactttgggaggccaaggcgggtggatcacctgaggtcaggagtttgagaccagcctggtcaacgtggtgaaaccccgtctctactaaaaagacaaaaattagccaggcgtaatggcgggtgcctgtaatcccagctacttgggaggctgaggcaggagaattgcttgaacctaggaggtggaggttgcagtgagcttagattgcgccactgcactccagcctgggtgacaagagcgaaactccgactcaaaaaaaagaaaaaagaatgtcctatgaagccaggcacagtggctcatgcctgtaatcccagcactttgggaggctgaggcgggtggatcgcttgaggttgggagttcgagaccagcctggccaacatggtaaaaccccaactctactaaaaaatacaaagaattagctgggcatggtagcgggagcctatagtcccagctggtagggaggctgaggcaggagaatcgcttgaacccagaaggtggaggttgcagtgagccaagatcgcgccatcgcactccagcctgggcaacaagagtgaaactcagtctcaaaaaaaaaaaaaaaaaaaaagaatgtcctatgaaatatttggcacataaatacaggatgcccaggtaaatttaaatttcacataaactatgtacagtttattttatttattttattttatttttttgagacaaggtctctctctgtcacccaggctggagtgcagtggcacgatctcggctcactgcaacctccacctcctgggttcaagcgattctcttgcctcagcctcctgagcagctgggactacaggtgcatgccaccacacccagctaatttttgcatttttagtagagatggggtttcaccatgttggccaggctggtcttgaactcttgacttcaagtgatccgcccacctcagcctcccaaagtgctgggattacaggcatgagccaccgcacccagccaaacaatgtgcaatttttaagtataattacatcttgcaatatttgggatgtagttatgctaaaaaaaattcatcactcatttgaaattcaaatttaactggacatgctgtatttttatttgctaagtctggccatcagtatgtgatgctgaataaatgctacttaccagtgctgtgaactccagggcagagctccctcctgttacactaactagcctggagagttccgccaccagagagccaatttctcggccgcctttgggtgctgactcagctctctgatgacccaagtttatgagttacctgtgagaatatctgggcccccagttttctcaagagtcagccccagatccccaattctgcctctcaaaatcgaggaataggagccctggctttttccagcagcagctggaatcaataagcagccctgatgtgtggggtgggctggaacacagcctgctaccttgagccctgcagtattgatcgcacaggcagctgtctgtaaaccaagcaggcatgctgaaatggggtagtgttgtcagccttcactttcatagggatgactcttcagatttctctcaaagcctgtcgggctcaaagggaaaaccagcaagttcttttttttcactaatgtaatgccttggggagctgccgcattacattctttttttcttttttcttttttagtgcaaagttgtgggggtggaaggcagaaaccacaacaatcctctctcttttgggactctctctcttaaatgtggtccatcattcagttttaataactgaagcacaaaatatgaaaactaaaaaaagtcattttttatcgaaagcctcaaatgtactgggcatgagagcgagcagtttgtatgtcttgtggtgttttatgcttctaatgattctctgagattgatatttattactattcccattttacagattcagaaatggaggctcacccaggctaagtaatttgctcagggccgtgcgtcccgcagtgagtctgctatggtcgaaacccaggtctgtttgacatctaacactactgtctgctcaatgagcccaagacattgcaaacactgctggcttgtcatcctagcttgtccttttagattcctgagtgatgaacatcctctgagaaactgtccctggttgctctgaaaaaagccgggtacctccctctgctctcagatgactttcttcccacctgtgtgttgtagaacctccctgtggggctgtaaaaacacatccacctttctgtgtcctcattaatccacaaggtcctccagggcagcggcaaaattgtttttctaccaagtgtgctcctctctagctgcagggactagactatcgtttagcacacaagctctataaatatctgctcgtggttattactgttttgaaatctcttttcccatttgtttagggagcttcaaggatgctgtcctgtcaccatgtttcattgtgatgccggtcttctgcgttcttacctatgttttttacaccgaattttgtattggcctgtcctcattgaggactcatctggctggtccataccaatattaacagaaagttcttccatggttgaatgactgaacaagtatggttgccatgatgactatatttgcacaaatccaagttggtcttctcaatggttcttcctataatttggtagaagggtatagaggtaaatgaacccaatgggtagccaaattcctgcacctgggatctcaaggtcaatgtttttttttatcacagtgatatagggtgtctcttggagactcaagagaaagagggttgtcaatgagagagggtaaaagatgacagagtggctttgatcctaccttctcacaaaagcttcttagaaaagtcaacctacttttgagaggagcacaggttctcacaaaaccccttccaggaagtagagaagcaaactttgccatgattctgctaccaggagtaaacatgtgatcccacatgtactctgccccttcagggatgaaagagctatgaagctgatggttgatcctcagggacagacattggctgagaggacttgatggatttcagatctgcccacctgaagcccaaacgctgaggaagtctctaaatggggattatagcatttaaacattttttatctttatgttcttctgcaggtctgaggcccttcagactaacagtgatccccctgtcacattaattcttctcacatagactgtttttcttataggtcctatctggggcaaagatgactatgacagaaatctcagcttgttcttgctgacagagttaaatttggtctgtgtttgctctgcacaagaagaaagcctgaactgtgctgaacaactgtcttgagggcacgtccagagccacgtttgtctctctcatgttcctccttttattgtttttgttcctgtacatatttcagtgatgtcaggattttttttaagcctccaccttaggggaagctcaagaaatggtctgtgttagcactggtgaggccaagacagttttatgcatataagcctggctgtttgttgatcccttggaatgagaaaccaattggaatatcctggctgtgagaaatataacccttgaccagccaaagtcatttgtctgccttccctgttgaaagaagcacatttaaaatttctcacctgggagaaacttcctggaaagaatatttcatgcaaaataatacacttcttggacctgagttgcaatttggattagaaaaaaatgacctcattatgtctgagcatatatttttgttagaagccattctgagaacaagagcatgtggccgggtgcagtggctcatgcctgtaatcccagcactttgggaggccaaggtgggcggaccacttgaggtcaagagtttgagaccagcctggccaacatggtgaaacccgtctctactaaaaatacaaaaattagccagacgtggtgttgcacacctataatcctagctattcagggggctgaggcaggagaatcgctggaacccgggaggcagaggttgcagtgagccgagattgtggcactgcactaaagactggagacagagcgagactccatctccaaaaaaaaaaaaaaaaaaagggggcatgggttattcaagggattgttgagttgtttatacatgatctataccttggagtaggttttggatgcattttgctccacatcccgaatgaggggtttgaaggttgaactgctatgttgatttaatgctgtggttgctagggaggaggccccagaaaagttcagaatgttaaaatcaagtgttaaaatatgctgggaggaaatgcagaacagttctcaatttccatcatgagagctttatagatagttactggaagaaaaaaaacctccaaaaattaaagtcagagatttatattttgtacacattttgaattttggagaatgttacttcttcctccagagatcttgttttgctttttaaattagccaggttttatgaaatacattccctaaggaacgctgaagaaatttccaatcaagattcttagtttctagtgctttgagggcaacaattgtacccaaaaaatttctctctttcttagaatctgccacttccagaggcctgtgcttggaaggcaggtgaccaagtggcatttgggtaccttgctggagaatggatgttagcacctgctctggggtaactagggttgccagtcctgctgggcaattggttatgcatctgttgcagctgtacactttgcgaaagatggatgatggggcatgactgcagaaaccttagaaattaaaaaagatatattttatatatatcatcagggagatcttgagaatttgccattcagagtatgaccgggagaaacaagactattttgaatccacaggttcaaatacagcatagaagatgcaggaagtggtatttggcctgatgtttctctcaatgtcattgacaacaacaaggatatatgctaaaaccagatggcagtgccttcctgatttctactgaggaaagctgtgcttctcgaatgctaatgtgtatatgaatcaccagaggattgtattaaattgcagattttggttcagtaagtttgggtagagcctgagattcttgcttcttgagctcccaggtgatgttgaagctacaggggcaggatccacctcctccctcactcttgccaggagaatacccaacatctttccagtgtctatcagactaggctgagcacttcctctaagatctgctgaaccctggatactcaagtttgcaacacaaaggaaggaaccaaggggtagaaactcccctgggaagagacagtgttctctgtggtagaattggggagggaacccaaatggaagcagggaccccgaaggagggagctggatggagaaaacagggtattagcaagacaaaggacagggagggatgtctccatgggaagctgaagccctttacaacctccaccttccaaagactcaagtctgcaaggctgtccttctgcaacagaattcaatggttaaatgaaatctacttaggacttctatttctcactcatggaaatgctactttgagctatatctgatctttcaataaataactttctcctggcaccactataatctcgttatcatacctatatcagcaggacacagtggggtaccaacagaaaaccccttcattttctcttactgttgtctttcaaccccaatcctccctgaccttggacagtatctaggcccaagtgcatagcttttctgattacttccaggtaatgatgtacacacttatcatcagatattaaaaacttcaaaaacaacctgatctcaaaactgtaatctctcatcctatttcaagcttctcctctctctctagctcagaccagactcctgcagaaacctgcagggaaagaaggtgtgaacaattccatgtcctcccccgctgctctgctgcttcgggttcaggttggactgggagagggcagaggagcagcaaggggagtcaggagcttttcacctgagctgttggaatctgcagttaggaatctgccctctgtgtgacctgagtcctaactcagctgtctgctagttcccaggataattccaacaggagcacttaccaagtaacgacctatatcagcactcttctagatgtcacgggaaatgaacatcctcaaagtaactctcattgcagagtaattcagagttggcaatttgatttttctctttcttggtctcataaattgttctccctctaggaaagctgtataaattcccactaaaagtggcccttgccttgaagactggtttgcaggcatgagacctttccctgggttactggcagccctttaattcacgtatgaagcctctttgtcctgcagtcacgggtccaagaagatggctcaggataggcagccccacattttttttgctcaacatatatagatgactggaagtggaactattcaaactactaaaactttcagacccctctccagaatttctaggataatgtctacaaagtacattctaggtttagcataagctactatggggaaaagaaagaagagaaaaagagaaggaagaatactccccagaagtccaacctcagaggctccgtgggtttctggtcggactgccctgatgctcagaggacaaacatcaacttggggccagtgtgaggtgctgaccccttggccagatgagctggattatattcacgcctcgggtcctgctcacagagtgagaatgaacccttaatactttgctggattcacaagagggcatctggttttaaagattcttttgctgtgggttgaattgtgtctcccctatgctccccaaaaagataggaagtcctaacctccatactagagatgtgatcttatttggaaatagggtgattgcagatgtaatcggtttaggtcagactggagtagggcaggccccgaatccagtaagactgatgtccttagaagaaggcagccctgggcagacagagacacaccaggagagctccaagtgatgatgaggccagaaccaaagaaagccacaggatgctgaaggcagagatgtaggaagggacaaagaagtatcctcccttagggctttcagagggagcgtggccctgccaacaccttgatttccaacttttggcctccagaactgagacatacatttctgttgttttaagcaacccagtttgtggtactttgttatggcaacccagggaaactaatacacctcatatggcccttacaggaggatggtaactgcatttgcttgcgagcatatctgatctcacgcaatagcgtggaagctcctaaggttccctggagcccattgcctaacacagaacaaatacagtgtgtgccagtaaattttggttgaatgaacaaatgtaggactttcaatcttaatggtagattaaatacaaacaaaatataagaattaacaagggagaaaggcatacctagattggtgtgtatatttcattcacaacaacattatataatcttgtttatgatttgcataaatgagctattatgccataatttgaaactcaaatcggtggcgtctccctgcaacatgaatgagatgaaacaagtcttcctctcctctctgcatgttaaagatgattgctgtgttagtccaatgatagatcccggtcttcactgtctcataacgtctcagcatcttttctggtttctcactggggctggaatagctgctgagggactcttcctgcctgtgagatttacattatttgagatttaaagcaaacgctatttcaaaactagcagtaacttcaataattggtggaagtgattgccccaaggtggaatctccggtcatcaatttaacttacctagttcttaatctccagaaaatagctggcctctgaatcattgctgtatacttaagaggccacgatagaagagtcccagggccttggaaaagatttgtggtgataatctcattgggactttatttcttcatggattcctttctgaatagcaacttagcacatacacaattgagagggataactgcatgtatgtgtgtgcatgtgtgttcatgcatggagtacaaccagcagaagagaaggactagaggacccatattgcaatcacttatgggaggatgcataagaaaatacaagggagataagaaaaagaaaacctaataaattagaattaattattaattgcaaattagtaaccaattcctttaataattttgtctctcttttcacaattctttatcttttgtatttaaagattctggttccatttggtgatattcatttgcttcttattcaaatgagatgaccagggatagtttgggggaattttttttggaccctgacaggggtcatgtctttcaatcctatccctaaattcatacccatatcttcttcagtccaaatttaattcagcccaacacatatgggatgatttcctatcttatgccaggctcagtgcttgacagttgaatacgaagatgagtaacacggctccagattatgaggagtttctaatctggcaaggggatgagatgcattacctaggtgtgacagggacctttcattttctgtccttatgggtgatctttgccaatccctggagggcaatcaagaggttaagacatattgcctcccaaatctggagtcttctctagcttctcccgggttgtgagtctcttctatgcagctgatcctacaggtgatctcaaaagcatcccacccaaagggttgtacaggcccatttctccttgggccaacctacattttagaccttgccaaattcaatacttgagactacaatttagacacatggaattctctaccgaatggctattggtttggagtttccatttgggttctaaccaattgagcaatggtgcaagcgttccccagggggaacaaaaagtgggatctattttattgtagatgcagcgagtgcccagtgacctcgagaacatggggctcctgggcaacgctggactgcagcattgtggggcccccagaaccaaaatctaccttccgattccttccaccagaatggaagttatgtggctcccagtgaatatcaggctgacctcattgagtcataaatgaaggagaaagatgagatagaagagaggggtgagcctgtttgggcttcccaataacaggcctgtctggtatgtgacccagcatggagcctgaatacaaaggacccatcctgcctgagcatagatgggccaagagagcaagacaggggctgaccctgttttctacttttaaagccaagcaccccactcactggcatgtgggatgtgatcccaagagtttctgcctctcttggatcacgaatcaagccagtttttttaagaaggggaaaacagtaaaatggggatccccccttttttttaagaaggggaaaacagtaaaaatggggatcaaaatgtacattcccacacttcctatcttggagtggaaatctcctttcctctatgaaaacctggatgtgctgttgggtaggggaggcagctgagaatgttcctggcaatgaggcaagcaaactactactagacaaaaacttgcaagactatttccaagtatgtagcagggattcgagttgaaattctaggtagacatttccttctcttgtaccttcccattttggccctgcatgaggaggctgtctcctcagatgttccgtgagcacttgtcctgaatttcttccctctgatcatccactctgcagctatctcctcaggtctgtgagtgcaggaacaccacatctgtgtagggaaaatgtcgtgcctctctacgggttgatttaagactcttgtgacctagagcttcagctacatgtgtaatgatgttttctgctgagtcaccgcagctgctgggtgcaggtgggctgagaactcctaaatgggacccagggaaaagctacaggtgggcttgtcaccttacaagtcatctatgaagttgcaccttgccttagagatcacacagcctgaccctataatcctacatggagaaaggggcatgtttccaatttgcctttcttttagctttgcctcttagggacagtggacacctcactgcagggcaaggaagcattggatcctcttctgcccctcccaggcctggctgccttatgaggccatcaggagaaactcgttgtcacagagtgagtgacgatgtcaagtgcggggcagtgccgtggattaaattgtttgtaggtcttctgataaaagcaggcaggaaagagggtctgcaggttgctccaacatagacacataggtaatagagcttattcaggggagggaagagtgagaccccacaggaagtatcaataagattttgaaggttttcaaagagtaaaaagctcaaacctaacaggcagtaagggatgcaagctggaggcagtgctgcgggaggttgggggaagcagatggaagagcaaggacctggggtgaaaaagcaaattcagcctcggtttgtgttcaggcaaagggagggtcagcttttggctgtggtatagaaaatcagtaaggaagtgagaagactggaaaaggcaaaggggctgaaagccaggctgcagagttgatatttcatacgggagtatcctgcagttattggaagagtctgaggagaatgatctgatcaaagcagcactttaggaaggctaatctgtcacagtgtgtaggagggactggaggagaaacaggcaggaaggcagaggtgggagaactgtcagctaggatgaggatgacgatggtgatgaaaagatgatgtaaatcgtgaaaaagatgaagatgaggatagcagctaagattcattgtttattaatgacattcattattctaagtactttgtatctgttatcttatgtaatcctcacaccctcctatgaggtgagactgttttacaaataataaaactgaaaaacagagaagtaacttgtccagggtaactagctaataagtggtggacctagggtacaatccaggtgctctaatttatttatccaggcaaaatgcaatgagctgagcaaaagcagagtggtggtcatgggaaggtggagatggattgggatgtcacagggttggaggccacctgatcagatgtgatggcaggagagaggtaggtggattgggagaaggaaaagtggaaagggccatccagcttattgccataggtcactggggacatagtgggaatatcaattaaaataggggtgcccagaagagggactggtttccaggtgaagatggcacaccagtcagccaccaccatctctgatctgcagtgctgcaattatgtctctttgtcccttacaatcattctttacatggcaactcctacatgatgtaaagcggatcatgttattccccattaacatccttcaacgtctccacagctctcgaatctaaaatccagtctccttactctggcagacagattccacaggagctgccccagcctctttctggcaccgtgtcatagcttgtggctgcatcactctaacccccaaggctagtaccttcaaatctctctatgctccatcatcagattgctttctcctccatgtgtcaatgtcagaatctttctctgggccaaatgcagtggctcacacctgtaattccagcattttgggaggccaaggagggaggacggcttaaacccaggagttcaagaccagcctaggcaacatagtgagattccatctctacaaaaaattttgaaaaaattagctgggagtggtggtgcacacctgtagtcctggctacttgggaggctgaagcaggagcatcacttgagaccagaaggtggaggctgcagtgagccatgttcacaccactgcactccagactgggctagacagtgagacctcatctcttaggagaaaaaaatctctctctgcttctcaatcagaaagatacttatgtttgtacttagggtccactctgagaatccaggataatcttcccatgtcaagacccttatttagtcaaatctgcaaagaccctctttccatgtaaggtgacatgtataggttccggggattaggacctgatattttatgggaaccattacctagcctcctataatttctgctgccctccacttactctgtgcgggaaatcagctgtaccctaggtgcctagaagggtaccaggcacattgtagatgctctactgtgatttattaaactagcaaataaatgaatatagccaagtgtattttattctaaaaagtattttccctatatgttcataatttaataacaatacatcatattgttttaattctaaaacagtcaaacagctttgatacgacagtcatcttatataaatgaaaacgtgatctaatgaaatggcattttatgagctttaggcgaagcaaccatttgaatgatcttgtttaaattaaatggcccttttgtgttcagtagtcagcatcactaagtgtgtctatcttgctaatacagtggcatcattataatctaaattatacaatttaattacatgtaacttgaagctgaaatattggtgaggtacaagtatgacctttacatcacttattaatgttttttcaaatgacatgacactcagaaaaatttaacaaaatcttatgtttttgaatcggcttatgaatcggctaaataaaaccttaagacactgacaaacccagttccaacctttgctttgccatttaatggctgcatgatcctgggaaagatatttgagtactccaagattcacagtccccactctatagagttcttgagaagagtgaaatgagttaagcatctaaaatgtgcctcccaattactagccaatattagctccctcttcctccttcctttgccacaaacataccaggaaaaataaaattctgttttagatctgataaataattcctgaaacccagcatagcaaacataaacacactgtcctaaaatacatgggcagcatagtgtgtagccatcatgagccccgcctgcttgctttcccactgcattatgccctgagaaattgccccatgtgttttctggtgtttgattctgtgccagagccattagggtagaagaatgagaaggagttgagttcagtcctttcagagaggacactctcctttgggcatctgctggttatgagggagcttcttgccacaattcaggctgcatgctgctggcagagggaacgaagggaatgcactgtcagaaggacagagagacagagaagcagacagaagcatggcgatggccaagtgttctttagggagaaacaggctacatacgggataagatttttggaacatgaaagaagggttactttaccaatctgaggctgctcaggcagtggtgccagaagtcacagaacatataaagcagagtttttcctcatgcagtcatccctcaagatccatggggtgttgcttccaggacccccttagataccaaaatccatggatgctcaagtccctgatataaaatggcacagtatttgcatataacctacacacatgctcctgcttaatttaagtcacctatagatcactaacacctaacacagtgtaagtgcgatgcaaattgttgttgcattgtttaaaagttttatgattttttcttattgatatttttaattgttttttcaaatatttttgatccgtgcttgattgaatccagattgcggaacctatagatagagagggatgacttgtttttttccttctgttctccctttattccagctcattatttatttcatagaatctcaggtttggagaagtccttagagatattttggctcaacctccttctccatgcagaaatctcctttacaacattcacgacagatggccattgtggtgttgctttgttataatttgttttatttagtaattggtttttatagaacaccattaagttaaacagtttatttttcaaggctccatggaaatggctctctttaccatctgtgtgtgcctctcatatgagattagaccctgggactttctgccccttgatggcctgaactattagtaagggcagaaaagggaaaaggacttgcctcacttctcaatcaagactcctgggtaagattcagtgccagggaacggggagggaagggtagcaatgatttaaaagatatttactagcttgagaaaggtcattctgaccttcggtgttgtttctttaaaaataatactatttgagattttctgattatgcaagtaacccaggtgcacaaacttttaaaaattattctaaaagcacacaaaattactaaatatttgggagttctgcgatgatactgcttcttcagttttatattctgccctttttacattatagtgtgtcattacatgttcttccataatataagtcctagttatgtatcatattctgtttcatagctgtggcattatttacttaaccagccccttctggtttcctctcacctcttgcttcctcatgctggtcagaaaagctgcctctggcataagcacttcttaatctgccttgactttcatccagacacataaacgcttgtcccaatgcatttggatggaatgatacgattccagagtgtttcctgggagtaatcatggtgttgctgcggagtccatctttgggctttgtgtagttgatgtgttaattacactctgtcaagccacaaacttctattccccaagcctctccaggcatgcacaatcatctgtgtgtcacactgcaagtgaggataagtctaaatggaccctttaaatcagcttggctgccaactttggctctattgacttgtcagggcccaggcaatggcacagccagcagtcttggcccatattgaccagaatgggctgcagcagaactaccagcctcgcatcctgtgacaaacgacccaggacaatgggtggtacctgggctcccgctctaggagcaggcagtcctgctaacataccaccgcttccacccctgtctggctctctgctggccctgatccaaatggggcattgtgctttgttattgttgccataagaagtggcagcagggcctggaaatgcaaggtaaacatttgcaatgggattccaggggcatctccatggcctgatgctttgaggaggacagaaataggaatactaatgtcccagaatctgaatctgtctagaactgactgcttattgatgtgggttattttgctctgtaaagggacacacacacatacacacctcaaccttttagaagaaatcctgggttaataatactccacctgctggttccatctctgagttccccattcttggtcccatttggtcctgactgaactgctgaccatgaggctgacctcggcccttggtccttggtccttgacagtcttgagtctgatgacctccacctcctcttcacatggctccacattgctggatattgtcatcttgcttcctctcacatcctttgctagatatgactgctggtttcaagaggggcccttgggtgtcaggctaggagttgttgcataaaagaatcccctttgggtcttctttgtgatgtgctttgtcactgggacttaagcagtgctgcttgtgctctgagtctcatgacagcaatgcccaggctttagcatgtggctctggagggagcaggcatgggccttcaggaaccagagcaggaaaggaagacaaatgatgatgctgtggtggcctgtggtagcaattgattctggctttccgctctctcctccctagttacagaccttgtcttctagctgaaaagacaggctcctgatccaggcctagccaatcacagtgcccctttccctggacacagcaacagcccaggcgtgggtgtgagtcccacacagagttcatcagagtctgtccctgggattgattcatagatgttgggagagaaaggtgcttcctttccactgggtttgctaagcaagggctgccatgtggtgtgccatgggttacagagaaaacaaagctggtacataaaagagagcagagacagacaaaaatgagagacgggaaaagagaagcagcacagcagaggtgctgagcactagcatggccctcgaggctgctgtgccctccactgctacttcaattacatgaaaagctgcagcatctttctcaacaatgggagcccattattctctcgtctttcataagcgagtttgtgatgggctctgacacacatggccgaaagggccctgcctaatgcactttctgatgccagcaggtgccagtttgatttgtgaatacagatgagacatccctccaggaactttaggaaatagcataaggacactttgaggaagacccatttcctgcaggcaagcaggatgaagtcttctgccataataatttggattataaaagggaagtatgaccttgtttggatatcataagctggtgagccagaaatatgtaggctattctctcaaaaattcatacacatctcttaccagattggcttctggctggacctcctgcccccggcccaggccccaggccatcagacctgcttagaaacttgtctgaaactgagccatggaggtcgcctgagctggatgcaccatctacctcacccaactcaacagagagaagttcctgcaagagaattgtgaggttttagtggacaggtgaatgtagagcaggagaaataatcaggagtagcatcaagattggagactggggaggcaggtgtttggtgaatccattaacctagagaaaagattattaagggaagaggaggtatgtgggagagactaacttagagttagatgtattacacacacagtaggtgcagaacgcctggctctactgtgtctgctgtttcaggaggtgcttggaaacataggtctccctctcagaaaagagctccaggccagcacatacacatgtgtatgtgtgtgtgtccccttacagagcagaataacccacatcaataagcagtcagttctagacagattcagattctgggacattagtattcctgactatttctatcctcctcaaagcatcaggccatggagatgcccctggaatcccatcacaagtgtatgtgaagtagaagtagacgtcactggagtgtaggtgaaatagaagccatgagggttgaataaaattgcttaagaagagcattatattggtttcccagggctactataacacattaccataaagcagggtagcatgaaacaacagaaatttattctcacacagtttcggaggccagaaatttgaaatcaaggtgtcagcagggctgtgctccccctgaagtccctaaagaaggacccttccttgactcttcctagcttctggtagttgctgtccatccttgccattccttggcttccatctgcaccactcctatcgctacctccatagccacatggccttcttccctctgtgccttcctatctctgtgtgccctctcctcttcttacaagcacattggtcacattagatctagggcccactctaatcccataggacctcatcttaactatttatatctgcaagatcctaggtccaactgaggtcacattctgaggttctcagttggatctgaatttggggttggggagtgctatttaacccactataatggtgaagagagaaaaagagaaagctaaaggcagaatcatgagacgcacgaacattctggtcgttagtagggaagtgttagcaaaggaaagagaaagagagagagagagaccacattcctagctgctgagaggccgggtggggatgcaagcaggtaactgtgtgtgaccatcagaaaacactggtaacggcagttgggtgactaactaatatatttcacccaggactagagtggcagtccaggatgcagaactttcggtgctaaaacccaacagtaccaagcacactggaatggttggacactctacctgggaaccagcagttctattagaactgaatgtggggagaacgcacggctgaagcttaatggcttggggaatgagtaagccttaagaaatgtacttgcctggggcgtgattctggttgcgttgtttttctgacaattcaaccacatttggaattttcttacgtaggatatgggcttgagtttttaatttaaagtcttcgggtaaggtcagctcagtccctaaccaccggacatcgtggaacaaccaagttattggagattaaagtgtccttcaactgtcatgaatgccactctgaccctccttgtctggaggggacctccagggcatgctatttaaatgaacccaattttgaaagttcacctatctctactgaatgtgatagctatcttgctctgccttcagagcacttatcttcaaagtattcagcgccaggcaaaagccttgatttttccttgaaaaagaaattgtcaggatgtctgaatccatccagacctttgcatgtgtgatttcattattcatagtcagcattcctcttctggcttttgatctgtagtttacagaatgtgtgcataaggaggctctctttcccataaactgatctctttaccctctcagcccggaataactttccatttattcttttgataaagcgttgatgccgtgagccagcatacattaaatttatcatgatacaaacacagcactacactcttatccagcccagatcagcaaccagatcactgccagtgcccctcaagtgtctctgatgatgaacagcaaaggggacctgtggaatttctcacgaccatcaaatctgaagaaggaattcttctgagagcagtttgcaaagtctatcaccagtatcctgactctattattagcttgcaaggtagttaaagatatcttctcagccttgactcactaataagcacttttaaatatatatcaaaaagccctcttaattaaaatgcttcaactgctttctacctctcttctgataaatgccaatatcattaacatggctgctcagaaccctgcctgcctctccagtcttgcctggccccagactcctctttcccttctgctacttcccctaagttgaatatttttcattcttctttcacctgcttatctcctactcatccttctcttgtcatgtcaattatcactttcttgaggaagcttttcctaacccaccccatgtcccatccacctacttgcccctgatccccaggatacgttagagacctttgctttcacagaactgctaccaagtcaagataggctgacatgtgaccatagcaagcaccaccaaaacctcagggtggatgaatgaggatccacactgcttgagacactcagcttccaaggccaagtccatgaacctgccctctcctgggagcgcacggtgtatacatgtatatagatgtgtatttataacccaattattgtaacaaattatttattagatatagacatccatatcttatctacatttcaagatagaagtactttggaaataggaagatactgtgtctgtttcacctttgagtctcccactgtgcctataatctgcactgacagacatgagatgttccataaaggtcaattaattaatgaatgaaatcatgagcaaatgatttttgaaatcattttttcttacatatttttgagaaatgtaagcatctttgtgttttcccatcaatatttttagaaagcttcatttttcaacagctctgattaataatataattgaaaagcaaagaagtctttttaaatatgtactgtataagtctgtgatacttttgatcccacagagagtcaattccatttagttcaattcagcaagtgttaagtgcctatgatgtcaaatgcctgtagtgtggcaacagaatgcttcatttcagtgggataatttgggatgaaaaacttctaaacacgtttcagctcctaagtctgacttcagcagtcacaaagcaattctcccatgtttagactatagccattcattcatttgttcaataaaactttattggacatctattagatatgatcatgactgctctggattagatctcttgaaaactgtgatacaaaagaagagattgtgcaaggtctttcccacttggaaagaaaagttaaatagataaagcacctgagtaagagtaagcactgagtaaaagacctgggtttaatttcccacctcattctgttgttaagtagtgaagtgagcttagcaagtcactccatagctctgggcctcaatttgttatctgtgtagtaagggcagtagactggatgatgtctaaagaatattagagattagaaagtcattgtcctggatggctcacaccagtcactggagttgcttggcaggaaggcataacagcattttgtacatctagagctctgtccctctcatggggcaagtgatggaagaatccagtggcaaagttaggtttagcaggaagtcctctcccaataccaggggtctgagtcccggtgccatgctttgtttacctctatcacttgcaaggatgaaaaagcatctctaggcctggcccaatgtatggaatctaattcatgggactagatattttattagcatttccagctgaatgagtgatttggggttggacaatgtgacagtgaatagatgacatattccttaactagtgagtatgaaactgcatagaaataagatttcacaatgttatgatattaatgtactttctaatattttacatgatccatatgaagacatataatttacagtttagggtagatgtacataggtaacaaaaataactggagactccttttggagaagtctgcaattggatcaattagcatacattcccttacaagtgactcaaaacccaactagcaaagacggaaattttaggaacatttattgtttacccagcaagatgtctgaagagggtggttccaggtgtcagggcctggatttgtgtctctgccattttcttgctttttcccagtcacaagataactaacacagctgcagatatcaaatcctcacatgactgatgactgtaactacagaaaggaagcaggggagatgaacagacctctccctttgtacctctctcctagtattagagagaaaaacacctttccagaagcttccccagctgacctctcctctgtctcagcagcaaagaaagtttgagaaagcagaaatctgtggttttcagcctctactttggaaggaagacaaattaaaagcaagttggggatgtctgtggataggctgccaaagtgcttaccagaatccatcccacaggccacccaccatctgtagatatctttcctctcccacagccactgtgggtggaatgctgtggcccattgtctgagagagaactccacaggatcatccagttactgtagctagcaccaaatcttgcacatctgggtgatttgtagtctccatgccatcacatctcggtgtagattcctacggtgtagcagttgataactaaaagaaaatgtgtctgcttcaaatgcaatgatgctgaagtttaatttacctttcattaaaacttgcaattggaaaaaagaatgataggaaacacaatcgtcactgtgggtttttttccccccacatatctagttttgttgatctagaataatgaggttgagagaggattaaactccttggttagcccatctggcagcctataatactgatctttgggaaaatgttcctcattcatgtcctctgtgacccacctgagacattagagaagacgctccccttagaaattgcacagcctttccagctcacttcgttttagatcaaggttttgggatcttgatattgtcttaggagttaaacagagctcaatggtcatagcctgttgttgaccagccttagagtctatctggtcatgcaattgcttcaaaatcttagtgggctttctgatatctttgcttcaagtcaatttctgcaactaatgtgtctgtttgggttctttaggaagcagaacactgagatggagttaggagtgtaagcaatttatttttaggtggtgacacttatgaaagctaaaagttaaggaggctggattggaaagggagaaccttcagactatgatgtaaatctcacacctgtcacaagagatggggtagaagtaggattgggcaggagaatcctcagactatgacacagatctggcagaagtctttgccaactcaacagggaactgcagagcaaagacagctcattaagagagctacacattgggcagaaatagcccggaccttggatgcctactgtacttgtcattatctagaagctgctcagaaacagtatgatttccactccaaagtggagcaggatcccaaaggaactggcggctgtcagctaaccactctcactgcaagttctttctcgaagggagatctgattagggcacctccatggtggccatggcccaggatctggtctggctgcttcatctgtctcccctctttataggcttaatggtggttgttctgaagcaattcagaataagaggttgggtgcagaggctctattttgatcagacctttgccagcagacttgccttctttgcctgtcgaagaatgatggatgaggggagtttgggaacttgggaatgttgtcagtcttatttcttgttatgtctatactcccctgcttctgactccagcatgtctccaaattatgaaactctcagttaattcagacctcacactactaacagagaacatctcagaagagttccttctctttcctccctcctgacagattttctctaatctgtgttcatcttcttcttgcaggactttgccaaaagcttcaagaagtagccaacataactcaacatccaaaatgtctcctgccatttcccttaacactacagcctcagctggcacgtgattggaatctcaaggcatgacaagcaagagtccaacgcaaatgtcttgccgccacccagcaagtgttacagacatccagcctattctagctggaccctcactgctctctgccctgccacctcaactcagccattctgtattttcagatattatctgtgctgctctatttactgcaactaaattcaaaggaatgtgtttggctacaagtaacagaggataggtcaataaaaaggattttatttagtctacaagaaatctggacaaaggtggttccagggtaagtttagcacttccatgacatcaaggcaccaggttgttatcttagccagtctctggccttcccctcatggccacaagatggctgccacagcttccagggttatcctcacctaacaacatccaaaggcagaaaggagggtcacagaagagtgttatctttgcatgcctccctcctgtgatcagagaagagaaatccttcccagaggcctactggcatagttctgttatactgccttcagttgcaaaggaagcttgtaacatgaatctctggcattttcattctttagagtgggaggaggacaaaggcggagagcgatttggagcacagccagacagcagcgtttgccatgggatcatgcctattgatcttagttttccaacctccaacttggaaatagataaaccatctgtcttcccttcctggggagtagcaggttatgaataaattatgctgccaga

SEQ ID NO: 10 - DNA, 25 bp, Artificial Sequence
  Synthetic oligonucleotide
gccctgcctaatgcactttctgatg

SEQ ID NO: 11 - DNA, 25 bp, Artificial Sequence
  Synthetic oligonucleotide
cttttataatccaaattattatggc

SEQ ID NO: 12 - DNA, 52 bp, Homo sapiens
  variation (26)..(27): Nucleotides at positions 26 and 27 are a and g or are absent
tggccaaagcagtggttagagaaaannaaaatttgctatactgagaaactta

SEQ ID NO: 13 - DNA, 52 bp, Homo sapiens
tggccaaagcagtggttagagaaaaagaaaatttgctatactgagaaactta

SEQ ID NO: 14 - DNA, 52 bp, Homo sapiens
taagtttctcagtatagcaaattttctttttctctaaccactgctttggcca

SEQ ID NO: 15 - DNA, 50 bp, Homo sapiens
tggccaaagcagtggttagagaaaaaaaatttgctatactgagaaactta

SEQ ID NO: 16 - DNA, 50 bp, Homo sapiens
taagtttctcagtatagcaaattttttttctctaaccactgctttggcca

SEQ ID NO: 17 - DNA, 56 bp, Homo sapiens
  variation (26)..(31): Nucleotides at positions 26 to 31 are tttcag or are absent
ttaaattgtgattggacaaaatgttnnnnnntttctgtcttttttttttttttttt

SEQ ID NO: 18 - DNA, 56 bp, Homo sapiens
ttaaattgtgattggacaaaatgtttttcagtttctgtcttttttttttttttttt

SEQ ID NO: 19 - DNA, 56 bp, Homo sapiens
aaaaaaaaaaaaaaaaagacagaaactgaaaaacattttgtccaatcacaatttaa

SEQ ID NO: 20 - DNA, 50 bp, Homo sapiens
ttaaattgtgattggacaaaatgtttttctgtcttttttttttttttttt

SEQ ID NO: 21 - DNA, 50 bp, Homo sapiens
aaaaaaaaaaaaaaaaagacagaaaaacattttgtccaatcacaatttaa

SEQ ID NO: 22 - DNA, 62 bp, Homo sapiens
  variation (26)..(37): Nucleotides at positions 26 to 37 are tgtgtgtgtgta or are absent
acttcatggtgtgtgtgtgtgtgtgnnnnnnnnnnnntatatatcacattttctttatccgc

SEQ ID NO: 23 - DNA, 62 bp, Homo sapiens
acttcatggtgtgtgtgtgtgtgtgtgtgtgtgtgtatatatatcacattttctttatccgc

SEQ ID NO: 24 - DNA, 62 bp, Homo sapiens
gcggataaagaaaatgtgatatatatacacacacacacacacacacacacacaccatgaagt

SEQ ID NO: 25 - DNA, 50 bp, Homo sapiens
acttcatggtgtgtgtgtgtgtgtgtatatatcacattttctttatccgc

SEQ ID NO: 26 - DNA, 50 bp, Homo sapiens
gcggataaagaaaatgtgatatatacacacacacacacacaccatgaagt

SEQ ID NO: 27 - DNA, 20 bp, Artificial Sequence
  Synthetic oligonucleotide
tgaggaaagatcactcctgg

SEQ ID NO: 28 - DNA, 20 bp, Artificial Sequence
  Synthetic oligonucleotide
ctgtcctcatagaggcatac

SEQ ID NO: 29 - DNA, 20 bp, Artificial Sequence
  Synthetic oligonucleotide
ctgtgcccagtccaaagtac

SEQ ID NO: 30 - DNA, 20 bp, Artificial Sequence
  Synthetic oligonucleotide
agcctggatgacagtgagac

(19) United States
(12) Patent Application Publication
(10) Pub. No.: US 2015/0292016 A1
(43) Pub. Date: Oct. 15, 2015

(54) NOVEL MARKERS FOR MENTAL DISORDERS

(71) Applicant: UNIVERSITÉ LAVAL, Quebec (CA)

(72) Inventors: ALEXANDRE BUREAU, QUEBEC (CA); YVON CHAGNON, QUEBEC (CA); MICHEL MAZIADE, QUEBEC (CA); CHANTAL MÉRETTE, QUEBEC (CA); MARC-ANDRÉ ROY, QUEBEC (CA)

(21) Appl. No.: 14/703,089

(22) Filed: May 4, 2015

Related U.S. Application Data
(63) Continuation-in-part of application No. PCT/CA2013/050824, filed on Oct. 31, 2013.
(60) Provisional application No. 61/722,424, filed on Nov. 5, 2012.

(51) Int. Cl. C12Q 1/68

(52) U.S. Cl. CPC C12Q 1/6883; C12Q 2600/156; C12Q 2600/16

(57) ABSTRACT

Methods and kits for diagnosing a psychiatric disorder such as schizophrenia, schizo-affective disorder, bipolar disorder or Tourette syndrome, or a predisposition thereto, in a subject, are disclosed. The methods and kits are based on the detection of the presence of SNP rs1156026, and/or of deletions located in introns of the DNAJC15 gene, in a biological sample from the subject.

### CROSS REFERENCE TO RELATED APPLICATIONS

This application is a continuation-in-part (CIP) application of PCT application No. PCT/CA2013/050824 filed on Oct. 31, 2013, which claims the benefit of U.S. Provisional Application Ser. No. 61/722,424 filed Nov. 5, 2012, which are all incorporated herein by reference in their entirety.

### SEQUENCE LISTING

Pursuant to 37 C.F.R. 1.821(c), a sequence listing is submitted herewith as an ASCII compliant text file named “Seq listing-11229.338_ST25.txt”, which was created on May 1, 2015 and has a size of ˜70,000 bytes. The content of the aforementioned file named “Seq listing-11229.338_ST25.txt” is hereby incorporated by reference in its entirety.

### TECHNICAL FIELD

The present invention generally relates to mental disorders. More specifically, the present invention relates to the detection of a predisposition to, and diagnosis of, psychiatric disorders such as schizophrenia (SZ), bipolar disorder (BP) and Tourette's syndrome (TS).

### BACKGROUND ART

Schizophrenia and related disorders such as brief psychotic disorder, delusional disorder, schizoaffective disorder, and schizophreniform disorder, are characterized by psychotic symptoms. Psychotic symptoms include delusions, hallucinations, disorganized thinking and speech, and bizarre and inappropriate behavior. Schizophrenia is characterized by psychosis (loss of contact with reality), hallucinations (false perceptions), delusions (false beliefs), disorganized speech and behavior, flattened affect (restricted range of emotions), cognitive deficits (impaired reasoning and problem solving), and occupational and social dysfunction. Diagnosis is typically based on the patient's self-reported experiences and observed behavior. No laboratory test for schizophrenia currently exists.

Bipolar disorders are characterized by episodes of mania and depression, which may alternate, although many patients have a predominance of one or the other.

Gilles de la Tourette Syndrome or Tourette's syndrome (TS) is a neuropsychiatric disorder characterized by chronic intermittent motor and vocal tics with an onset in childhood.

The diagnosis of psychiatric disorders typically requires evaluation by a trained mental-health professional and usually an interview, administration of a variety of personality tests (and in some cases, neuropsychological tests), and gathering of background (including medical) information about the individual.

There is thus a need for the development of novel markers and methods for the detection of a predisposition to, and/or for the diagnosis of, psychiatric disorders, in subjects.

The present description refers to a number of documents, the content of which are herein incorporated by reference in their entirety.

### SUMMARY OF THE INVENTION

In a first aspect, the present invention provides a method for diagnosing a psychiatric disorder or a predisposition thereto in a subject, said method comprising determining the identity of single nucleotide polymorphism (SNP) rs1156026 set forth in SEQ ID NO: 1 in a biological sample from said subject, and diagnosing said psychiatric disorder or predisposition thereto based on said identity.

In another aspect, the present invention provides a method for determining the risk that a subject suffers from a psychiatric disorder or has a predisposition thereto in a subject, said method comprising determining the identity of single nucleotide polymorphism (SNP) rs1156026 set forth in SEQ ID NO: 1 in a biological sample from said subject, and determining the risk that the subject suffers from a psychiatric disorder or has a predisposition thereto based on said identity.

In another aspect, the present invention provides a method for determining the risk that a subject develops major psychosis before the age of about 26, said method comprising determining the number of thymine (T)-containing alleles of SNP rs1156026 set forth in SEQ ID NO: 1 in a biological sample from said subject, and determining the risk that a subject develops major psychosis before the age of about 26 based on said number of thymine (T)-containing alleles of SNP rs1156026.

In another aspect, the present invention provides a method for screening a subject for susceptibility or predisposition to BP, said method comprising determining the identity of one or more of insertions/deletions (indels) rs10569822, rs113886253 and rs146020684 alleles set forth in SEQ ID NOs: 12, 17 and 22, respectively, in a biological sample from said subject, and diagnosing said susceptibility or predisposition to BP based on the identity of said one or more indels.

In another aspect, the present invention provides a method for the differential diagnosis of SZ and BP (or of a susceptibility/predisposition to SZ versus BP or vice versa) in a subject, said method comprising: (i) determining the identity of single nucleotide polymorphism (SNP) rs1156026 alleles set forth in SEQ ID NO: 1 in a biological sample (i.e. determining which allele(s) is/are present in the sample at the polymorphic nucleotide position of SNP rs1156026 (SEQ ID NO: 1)) from said subject); (ii) determining the identity of one or more of indels rs10569822, rs113886253 and/or rs146020684 alleles set forth in SEQ ID NOs: 12, 17 and 22, respectively, in said biological sample (i.e. determining which allele(s) is/are present in the sample at the polymorphic nucleotide positions of indels rs10569822, rs113886253 and/or rs146020684 (SEQ ID NOs: 12, 17 and 22)); wherein: (a) the presence of two cytosine (C)-containing alleles of SNP rs1156026 (CC genotype) and the absence of a deletion in rs10569822, rs113886253 and/or rs146020684 is indicative that said subject is more likely to suffer from BP, or has a susceptibility/predisposition thereto; (b) the presence of one or two thymine (T)-containing alleles of SNP rs1156026 (TC or TT genotype) and the absence of a deletion in rs10569822, rs113886253 and/or rs146020684 is indicative that said subject is more likely to suffer from SZ, or has a susceptibility/predisposition thereto. In an embodiment, the method comprises determining the identity of indels rs10569822, rs113886253 and rs146020684 in the sample.

In another aspect, the present invention provides a kit for diagnosing a psychiatric disorder or a predisposition thereto in a subject, or for determining the risk that a subject develops major psychosis before the age of about 26, the kit comprising (i) at least one reagent for determining the identity of single nucleotide polymorphism (SNP) rs1156026 set forth in SEQ ID NO: 1 in a biological sample from the subject. In an embodiment, the kit further comprises (ii) a container.

In another aspect, the present invention provides a kit for screening a subject for susceptibility or predisposition to BP, the kit comprising (i) at least one reagent for determining the identity of one or more of insertion/deletion (indel) rs10569822, rs113886253 and/or rs146020684 alleles set forth in SEQ ID NOs: 12, 17 and 22, respectively, in a biological sample from said subject.

In another aspect, the present invention provides a kit for the differential diagnosis of SZ and BP (or of a susceptibility/predisposition to SZ versus BP or vice versa) in a subject, said kit comprising: (i) at least one reagent for determining the identity of single nucleotide polymorphism (SNP) rs1156026 set forth in SEQ ID NO: 1 in a biological sample from the subject; and (ii) at least one reagent for determining the identity of one or more of insertion/deletion (indel) rs10569822, rs113886253 and/or rs146020684 alleles set forth in SEQ ID NOs: 12, 17 and 22, respectively, in a biological sample from said subject.

In an embodiment, the kit further comprises a container. In an embodiment, the kit comprises at least one reagent for determining the identity of indel rs10569822. In an embodiment, the kit comprises at least one reagent for determining the identity of indel rs113886253. In an embodiment, the kit comprises at least one reagent for determining the identity of indel rs146020684. In an embodiment, the kit comprises at least one reagent for determining the identity of indels rs10569822, rs113886253 and rs146020684.

In an embodiment, the above-mentioned psychiatric disorder is major psychosis.

In an embodiment, the presence of one or two thymine (T)-containing alleles of SNP rs1156026 is indicative that said subject suffers from major psychosis, or has a predisposition thereto (or has a higher risk of suffering from a psychiatric disorder or having a predisposition thereto, relative to a subject not having one or two thymine (T)-containing alleles of SNP rs1156026). In an embodiment, the presence of two thymine (T)-containing alleles of SNP rs1156026 is indicative that said subject suffers from major psychosis, or has a predisposition thereto (or has a higher risk of suffering from a psychiatric disorder or having a predisposition thereto, relative to a subject not having two thymine (T)-containing alleles of SNP rs1156026). In an embodiment, the presence of two thymine (T)-containing alleles of SNP rs1156026 is indicative that said subject has a predisposition to develop said psychiatric disorder before the age of about 26. In an embodiment, the major psychosis is schizophrenia (SZ) or bipolar disorder (BP). In a further embodiment, the major psychosis is SZ. In yet a further embodiment, the major psychosis is BP.

In an embodiment, the above-mentioned method further comprises measuring an episodic memory parameter in said subject. In a further embodiment, the above-mentioned episodic memory parameter is verbal episodic memory (VEM), visual episodic memory (VisEM), or both. In an embodiment, the episodic memory parameter is VEM. In another embodiment, the episodic memory parameter is VisEM. In another embodiment, the episodic memory parameter is VEM and VisEM.

In another embodiment, the above-mentioned psychiatric disorder is Tourette's syndrome (TS). In a further embodiment, the presence of one or two cytosine (C)-containing alleles of SNP rs1156026 is indicative that said subject suffers from TS, or has a predisposition thereto (or has a higher risk of suffering from TS or having a predisposition thereto, relative to a subject not having one or two cytosine (C)-containing alleles of SNP rs1156026). In an embodiment, the method further comprises determining the identity of one or more of SNPs rs1323142 (SEQ ID NO: 6), rs883877 (SEQ ID NO: 7) and/or rs9567343 (SEQ ID NO: 8) in a biological sample from said subject. In an embodiment, the presence of (i) one or two adenosine (A)-containing alleles of SNP rs1323142 (SEQ ID NO: 6), (ii) one or two thymine (T)-containing alleles of SNP rs883877 (SEQ ID NO: 7), and/or (iii) one or two guanine (G)-containing alleles of SNP rs9567343 (SEQ ID NO: 8) is indicative that said subject suffers from TS, or has a predisposition thereto (or has a higher risk of suffering from TS or having a predisposition thereto, relative to a subject not having the above-mentioned alleles of SNPs rs1323142 (SEQ ID NO: 6), rs883877 (SEQ ID NO: 7) and/or rs9567343 (SEQ ID NO: 8)).

In an embodiment, the above-mentioned kit further comprises instructions for using the kit for diagnosing a psychiatric disorder or a predisposition thereto, or for determining the risk that a subject develops schizophrenia (SZ), schizo-affective disorder (SAD) or bipolar disorder (BP) before the age of about 26.

In an embodiment, the above-mentioned kit further comprises at least one reagent for determining the identity of one or more of SNPs rs1323142 (SEQ ID NO: 6), rs883877 (SEQ ID NO: 7) and rs9567343 (SEQ ID NO: 8) in a biological sample from said subject.

In a further embodiment, the above-mentioned at least one reagent is one or more oligonucleotides.

In an embodiment, the identity of SNP rs1156026 is determined by sequencing a region encompassing SNP rs1156026 in a nucleic acid present in said sample. In a further embodiment, the nucleic acid is genomic DNA or cDNA, in yet a further embodiment genomic DNA.

In an embodiment, the identity of indel rs10569822, rs113886253 and/or rs146020684 is determined by sequencing a region encompassing indels rs10569822, rs113886253 and/or rs146020684 in a nucleic acid present in said sample. In a further embodiment, the nucleic acid is genomic DNA.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 shows a projection of the cases (darker grey “+”) and controls (lighter grey “x”) on the first two principal components defined from autosomal single nucleotide polymorphism (SNP) genotypes;

FIG. 2 shows the observed vs. expected distribution of −log₁₀ p-values in the case-control analysis of the SNPs in the candidate region. The results of the analyses under the allelic, dominant and recessive models are pooled together. MAF, minor allele frequency; HWE, Hardy-Weinberg equilibrium;

FIG. 3 shows the association to genotyped SNPs in the region defined around the linkage signal at 13q13-q14. Results of the Fisher exact test are shown. Mb: megabases;

FIG. 4 shows the association of SNP alleles and haplotypes with the narrow definition of schizophrenia;

FIG. 5 shows a global association tests with haplotypes consisting of three SNPs within 200 kb on either side of rs1156026. 1: rs1156026-rs2657099-rs1008913 triplet, 2: rs2120753-rs2657100-rs1156026 triplet, 3: rs2657100-rs1156026-rs2657099 triplet;

FIG. 6 shows the linkage disequilibrium between rs1156026 and neighbouring SNPs, measured by the squared correlation in the data from the 1000 Genomes project (Phase I version 3, Aug. 2012 update)

FIG. 7 shows the association of SNP alleles considering either all cases or early-onset cases only for schizophrenia, bipolar disorder and the common locus phenotype in the kindred sample;

FIG. 8 shows a distribution of the age of onset of (a) the common locus, (b) the schizophrenia and (c) the bipolar disorder phenotypes for the three genotypes of rs1156026. The distribution is shown for the narrow version of each phenotype.

FIG. 9 shows the genes at 13q14.11-13q14.2 that showed association with SZ (arrows) or surrounding rs1156026 (which is identified by a black square). Positions are in Mb (Build 37.3). DGKH: diacylglycerol kinase, eta; DNAJC15: DnaJ (Hsp40) homolog, subfamily C, member 15; ENOX1: ecto-NOX disulfide-thiol exchanger 1; HTR2A: 5-hydroxytryptamine (serotonin) receptor 2A;

FIGS. 10A and 10B show the receiver operating curve (ROC) curves for two logistic models: one with factors visual episodic memory (VisEM) and SNP (rs1156026), FIG. 10A; the other with only factor VisEM, FIG. 10B;

FIGS. 11A and 11B show the ROC curves for two logistic models: one with factors verbal episodic memory (VEM) and SNP (rs1156026), FIG. 11A; the other with only factor VEM, FIG. 11B;

FIG. 12 shows the results of the likelihood ratio test of the association of the 45 SNPs genotyped at 13q13-q14 with Tourette's syndrome;

FIGS. 13A to 13K show the sequence corresponding to nucleotides 43683486 to 43733602 of NCBI Reference Sequence NC_{—}000013.10 (or nucleotides 24663486 to 24713602 of NCBI Reference Sequence: NT_{—}024524.14). The sequence corresponding to SNP rs1156026 is underlined in FIG. 13J, with the C/T polymorphism between brackets;

FIG. 14 shows the prevalence of schizophrenia and bipolar disorder for each genotype category formed by SNP rs1156026 and deletion rs10569822 in the 10 kindreds linked to the 13q13-q14 region. SNP: single nucleotide polymorphism genotype, Del: deletion, SZ: schizophrenia, BP: bipolar disorder, OR: odds ratio, P=p-value;

FIG. 15 shows the prevalence of schizophrenia and bipolar disorder for each genotype category formed by SNP rs1156026 and deletion rs113886253 in the 10 kindreds linked to the 13q13-q14 region. SNP: single nucleotide polymorphism genotype, Del: deletion, SZ: schizophrenia, BP: bipolar disorder, OR: odds ratio, P=p-value;

FIG. 16 shows the prevalence of schizophrenia and bipolar disorder for each genotype category formed by SNP rs1156026 and deletion rs146020684 in the 10 kindreds linked to the 13q13-q14 region. SNP: single nucleotide polymorphism genotype, Del: deletion, SZ: schizophrenia, BP: bipolar disorder, OR: odds ratio, P=p-value.

### DISCLOSURE OF INVENTION

Terms and symbols of genetics, molecular biology, biochemistry and nucleic acids used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like. All terms are to be understood with their typical meanings established in the relevant art.

The articles “a” and “an” are used herein to refer to one or to more than one (i.e. to at least one) of the grammatical object of the article. By way of example, “an element” means one element or more than one element. Throughout this specification, unless the context requires otherwise, the words “comprise,” “comprises” and “comprising” will be understood to imply the inclusion of a stated step or element or group of steps or elements but not the exclusion of any other step or element or group of steps or elements.

As used herein, the term “predisposition” or “susceptibility” refers to the likelihood to develop a disorder or disease. An individual with a predisposition or susceptibility to a disorder or disease is more likely to develop the disorder or disease than an individual without the predisposition to the disorder or disease, or is more likely to develop the disorder or disease than members of a relevant general population under a given set of environmental conditions (diet, physical activity regime, geographic location, etc.).

The term “polymorphism” refers to the occurrence of two or more genetically determined variant forms (alleles) of a particular nucleic acid at a frequency where the rarer (or rarest) form could not be maintained by recurrent mutation alone. A single nucleotide polymorphism (SNP) or single nucleotide variant (SNV) results from a single base difference between related alleles at the same genetic locus. Exemplary nucleotide polymorphisms include the C/T polymorphism of SNP rs1156026 set forth in SEQ ID NO: 1.

The term “homozygous” refers to an individual having two identical alleles of a certain polymorphism. A non-limiting example is an individual having the TT genotype of SNP rs1156026. This individual is referred to as a homozygote for this SNP. The term “heterozygous” refers to an individual having two different alleles of a certain polymorphism. For example, an individual having the CT genotype of SNP rs1156026 is referred as a heterozygote individual for this SNP.

As used herein, the term “psychiatric disorder” refers to a mental disorder or illness that interferes with the way a person behaves, interacts with others, and functions in daily life. Examples of such disorders include, but are not limited to Anxiety Disorders, Attention Deficit Hyperactivity Disorder (ADHD), Eating Disorders, Manic-Depressive Illness, Schizophrenia (SZ), Schizo-affective Disorder (SAD), Schizoid Personality Disorder, Schizophreniform Disorder, Schizotypal Personality Disorder, Tourette's Syndrome (TS), Obsessive Compulsive Disorder (OCD), Panic Disorder, Post Traumatic Stress Disorder (PTSD), phobias, borderline personality disorder, Bipolar Disorder, sleep disorders, Acute Stress Disorder, Adjustment Disorder, Antisocial Personality Disorder, Asperger's Disorder, Avoidant Personality Disorder, Brief Psychotic Disorder, Bulimia Nervosa, Conduct Disorder, Cyclothymic Disorder, Delirium, Delusional Disorder, Dependent Personality Disorder, Dysthymic Disorder, Generalized Anxiety Disorder, Histrionic Personality Disorder, Major Depressive Disorder, Paranoid Personality Disorder and Shared Psychotic Disorder. In an embodiment, the psychiatric disorder is a psychotic disorder, for example Schizophrenia, Schizoaffective Disorder, Schizoid Personality Disorder, Schizophreniform Disorder, Schizotypal Personality Disorder, Shared psychotic disorder or Paraphrenia.

In the studies described herein, the present inventors have shown that SNP rs1156026 is associated with psychiatric disorders. More specifically, they have demonstrated that the T allele of rs1156026 is associated with SZ and with early onset (e.g., before the age of about 26) SZ, BP and SAD, whereas the C allele is associated with Tourette's syndrome (TS). Also, combining measures of episodic memory with the SNP rs1156026 T allele was shown to improve the prediction of SZ.

The present inventors have further shown that three deletions located in introns of the DnaJ (Hsp40) homolog, subfamily C, member 15 (DNAJC15) gene, represented by indels rs10569822, rs113886253 and rs146020684, were significantly associated with BP, in particular in subjects having at least one T allele at SNP rs1156026, and that the combined detection of SNP rs1156026 and these deletions may be used for the differential diagnosis of SZ and BP, e.g., to determine whether a patient presenting a first episode of non-specific psychosis is more likely to suffer from BP or SZ.

Accordingly, in a first aspect, the present invention provides a method for diagnosing a psychiatric disorder or a predisposition thereto in a subject, said method comprising determining the identity of single nucleotide polymorphism (SNP) rs1156026 alleles set forth in SEQ ID NO: 1 in a biological sample (i.e. determining which allele(s) is/are present in the sample at the polymorphic nucleotide position of SNP rs1156026 (SEQ ID NO: 1)) from said subject, and diagnosing said psychiatric disorder or predisposition thereto based on the identity of said SNP.

In another aspect, the present invention provides a method for screening a subject for susceptibility or predisposition to a psychiatric disorder, said method comprising determining the identity of single nucleotide polymorphism (SNP) rs1156026 alleles set forth in SEQ ID NO: 1 in a biological sample from said subject, and diagnosing said psychiatric disorder or predisposition thereto based on the identity of said SNP.

The present inventors have shown that the age of onset in carriers of the TT genotype of SNP rs1156026 was on average approximately 5 years earlier than in carriers of the CC genotype for SZ, BP and for major psychosis globally, comprising SZ, BP and schizo-affective disorder (SAD). Accordingly, in another aspect, the present invention also provides a method for determining the risk that a subject develops early onset schizophrenia (SZ), schizo-affective disorder (SAD) or bipolar disorder (BP), said method comprising determining the number of thymine (T)-containing alleles of SNP rs1156026 set forth in SEQ ID NO: 1 in a biological sample from said subject, and determining the risk that a subject develops early onset schizophrenia (SZ), bipolar disorder (BP) or schizo-affective disorder (SAD) based on said number of thymine (T)-containing alleles of SNP rs1156026. In an embodiment, the detection of two thymine (T)-containing alleles of SNP rs1156026 is indicative that the subject has a higher risk of developing early onset SZ, SAD or BP relative to a subject in which no or one thymine (T)-containing alleles of SNP rs1156026 is detected.

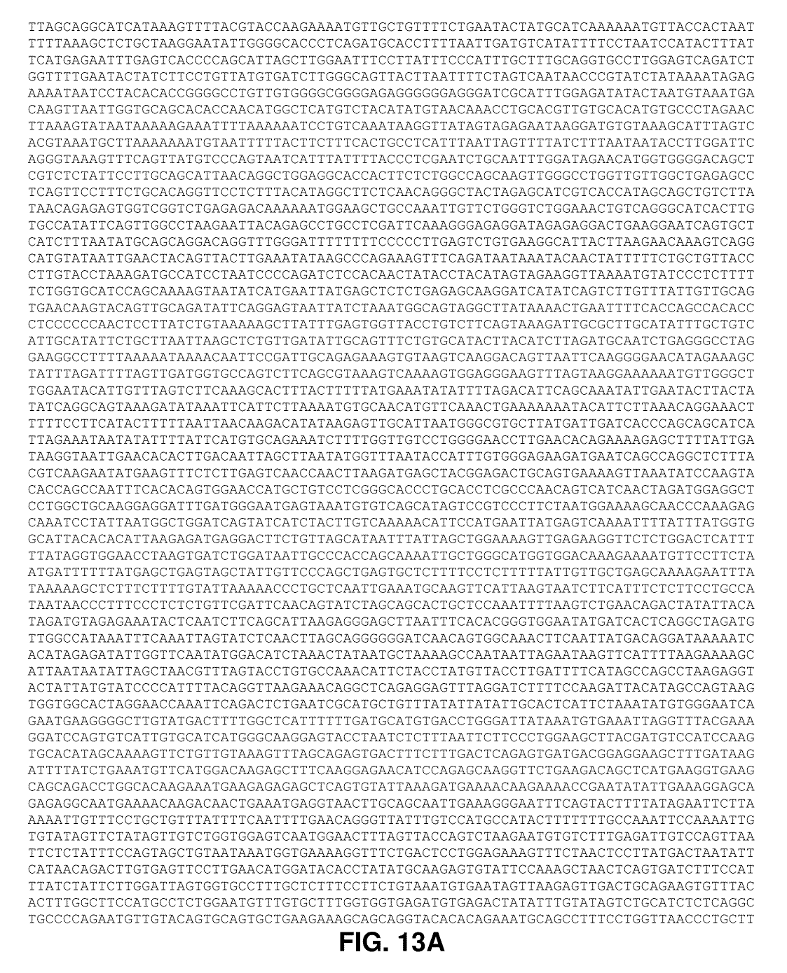

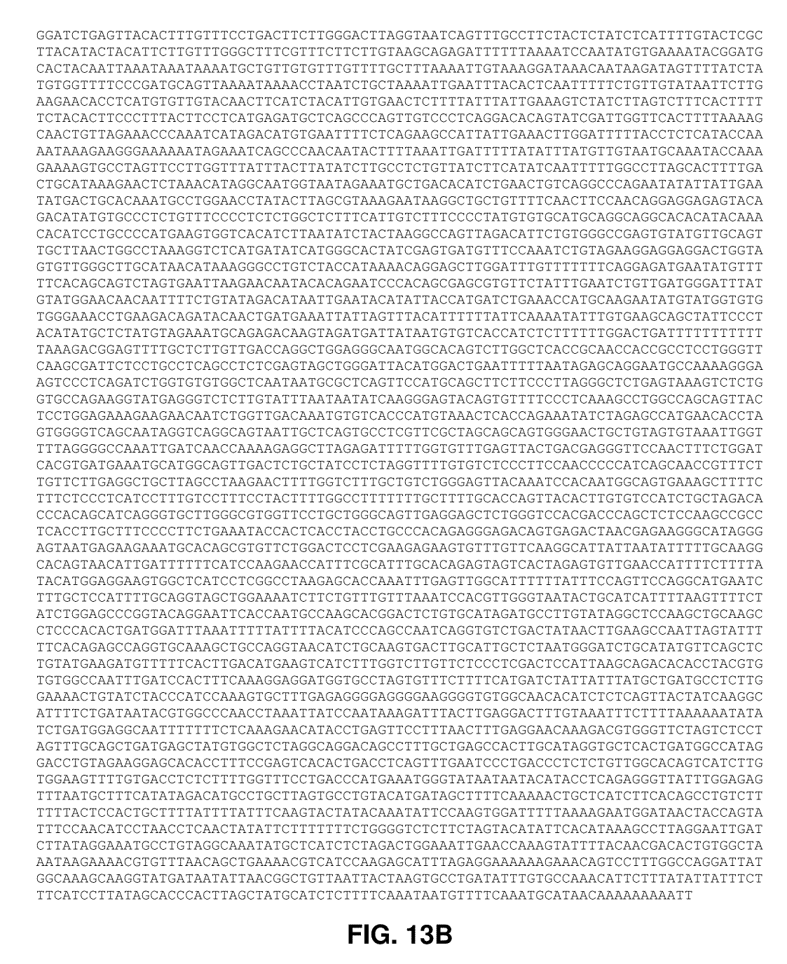

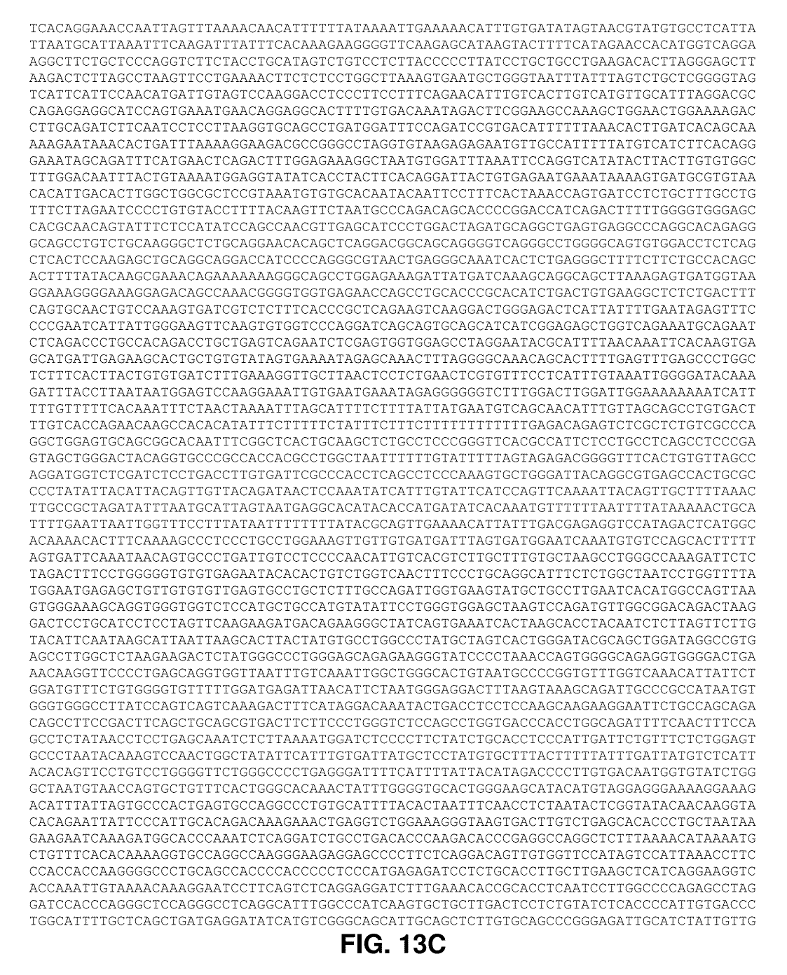

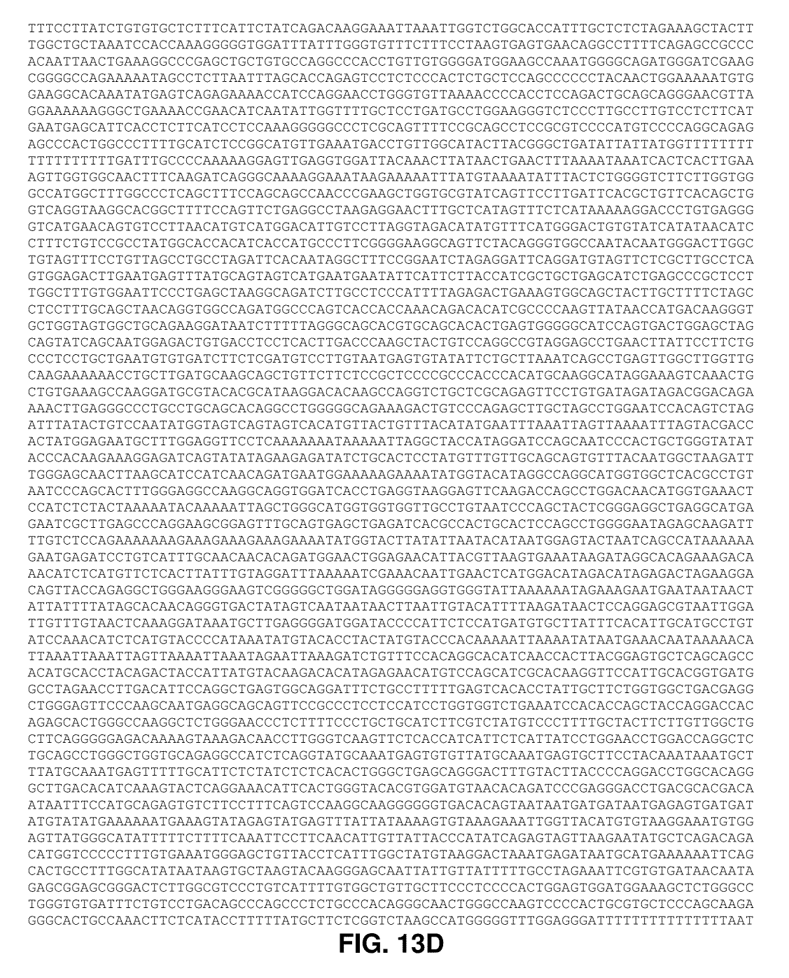

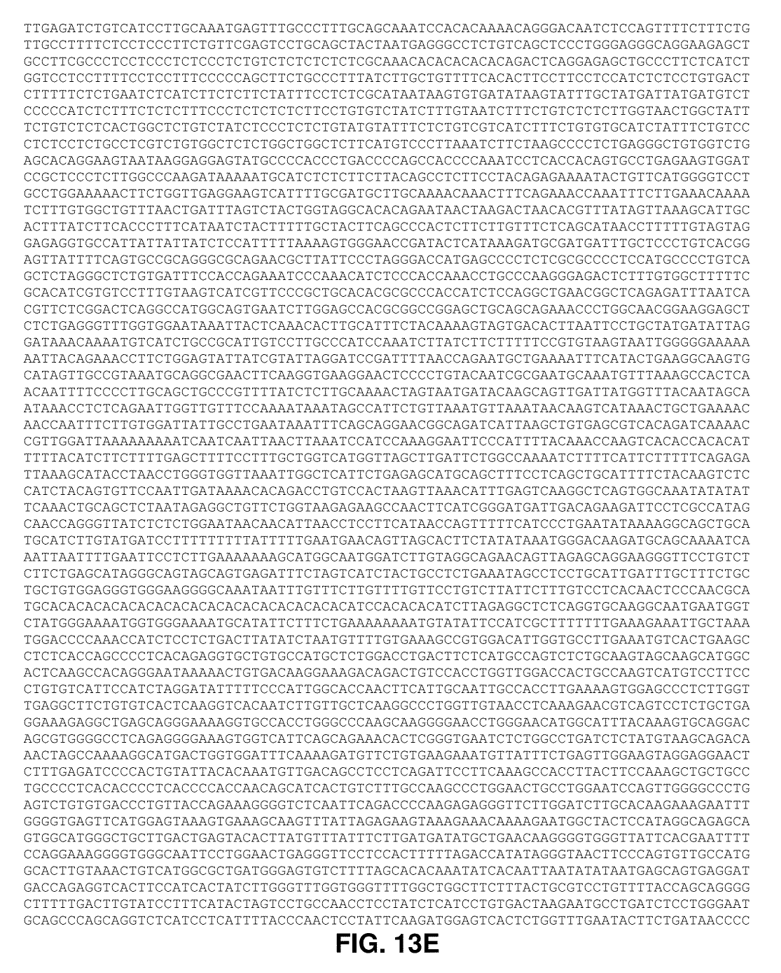

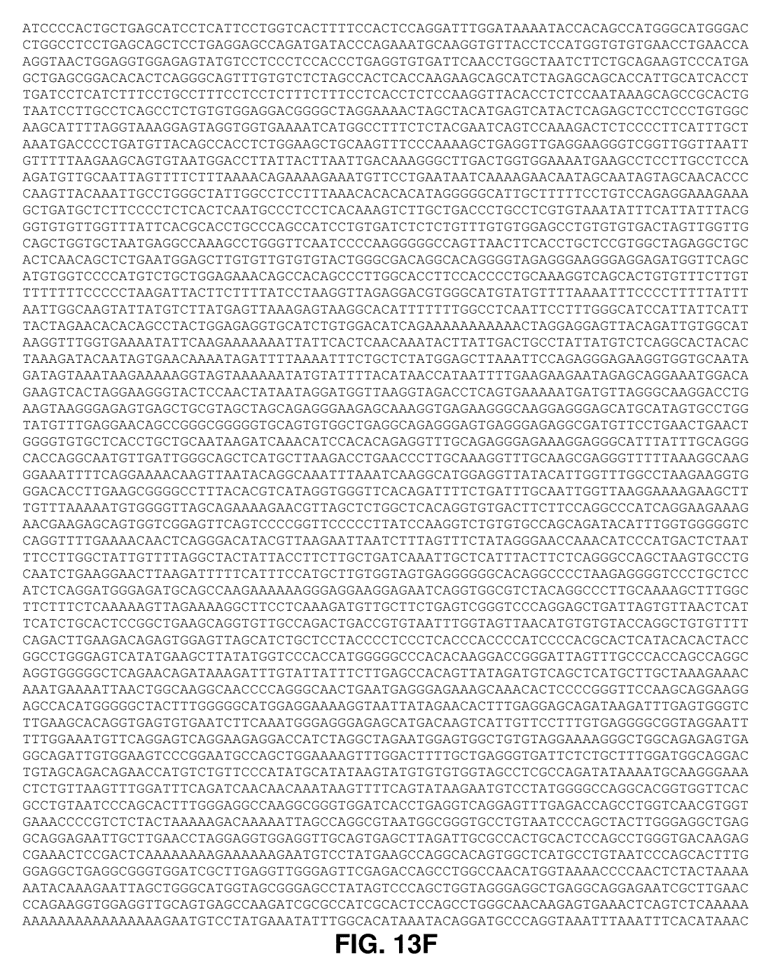

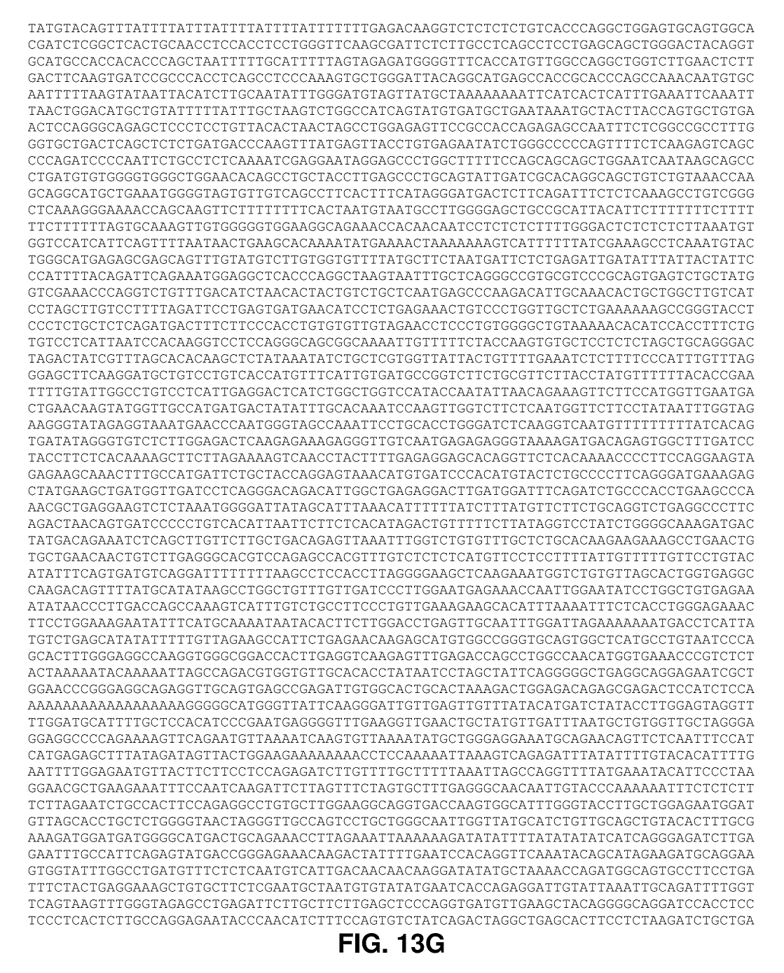

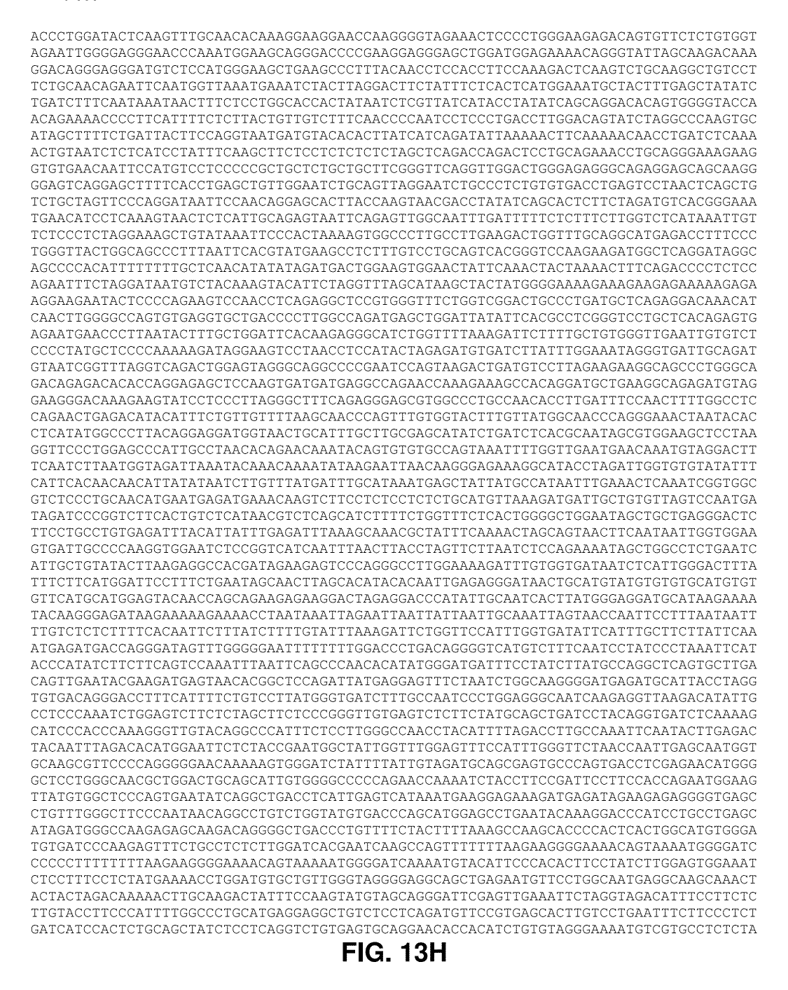

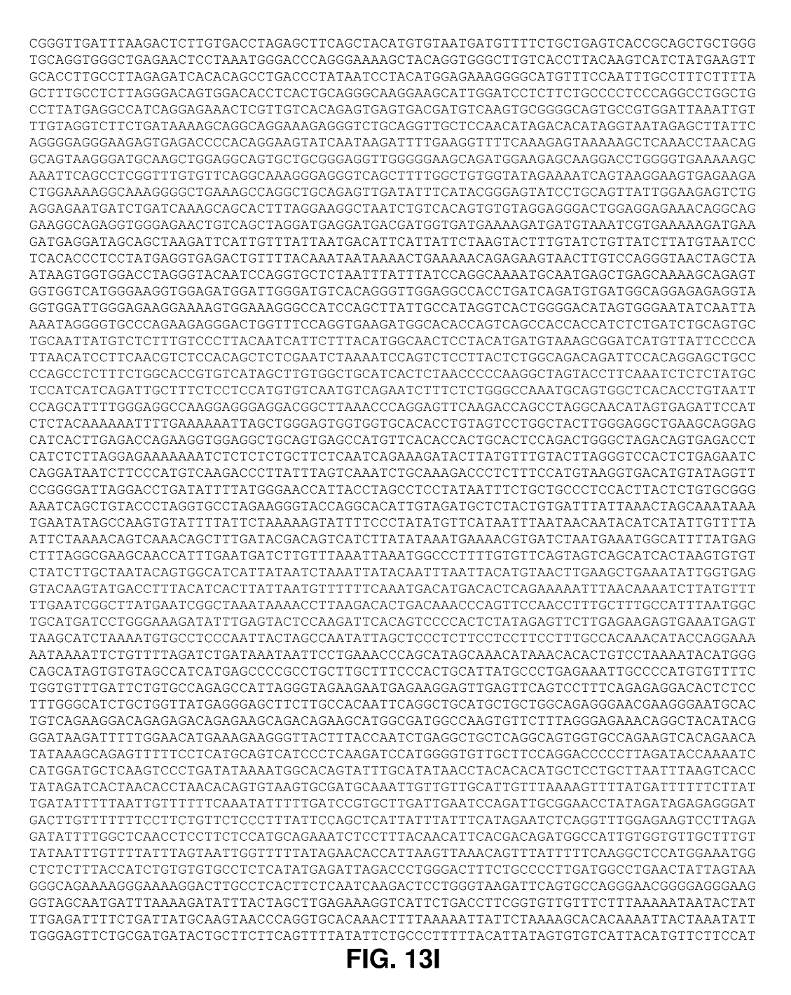

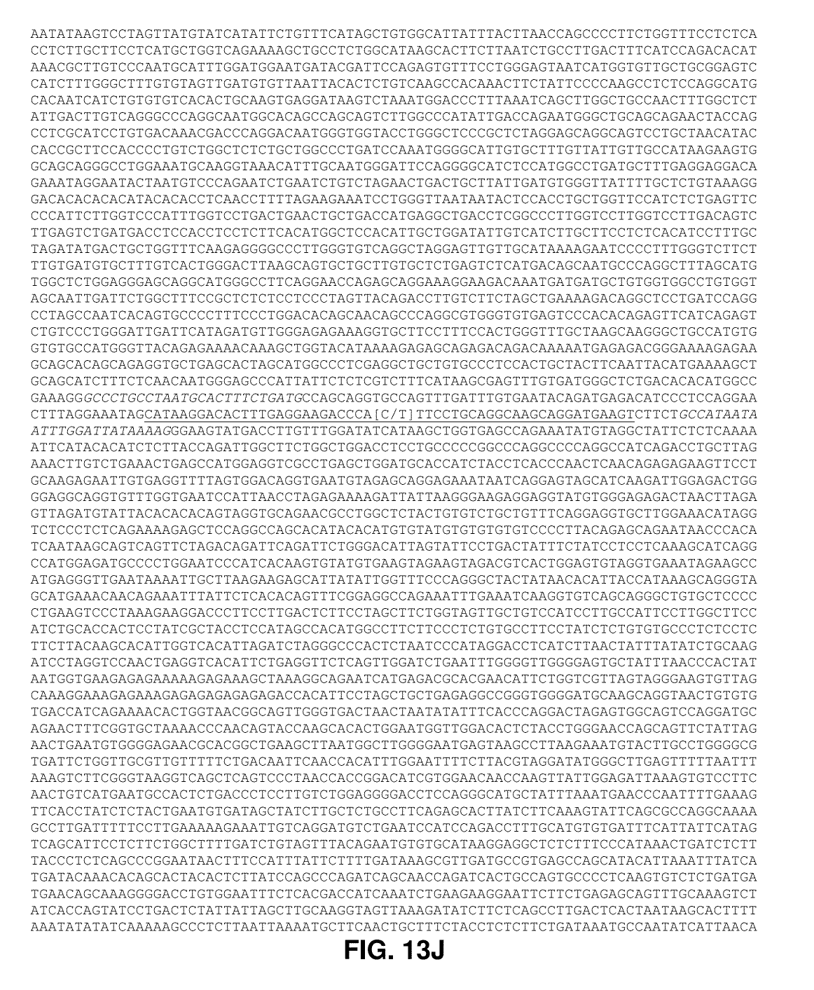

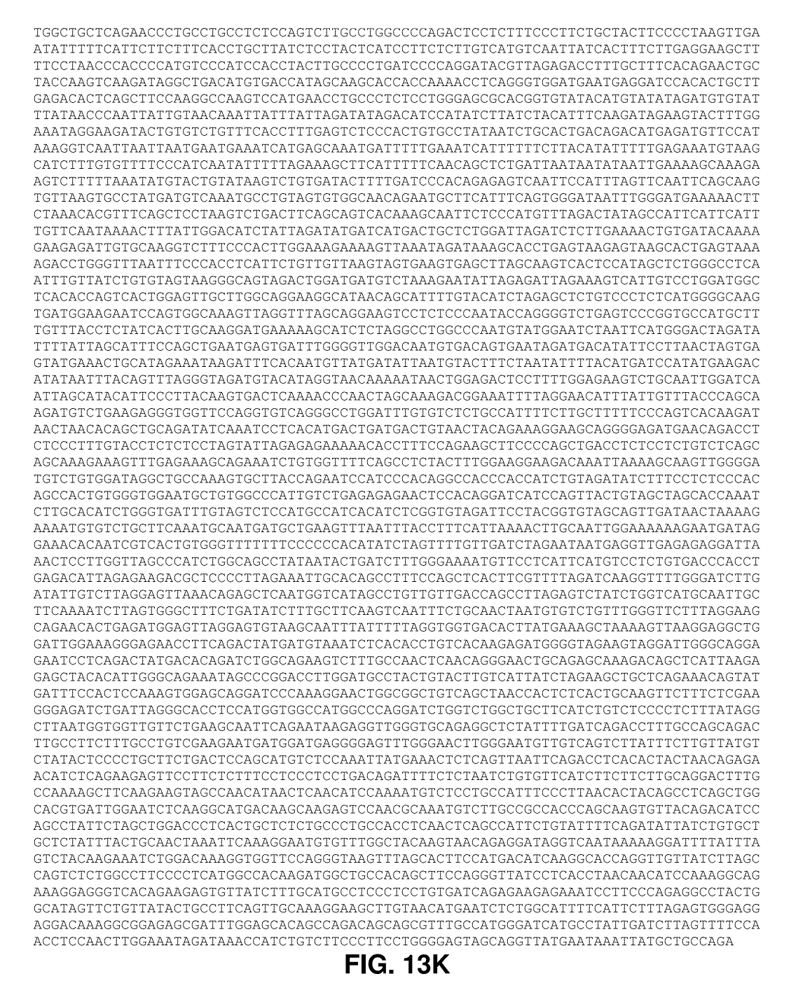

SNP rs1156026 (which is identical to SNP rs58530358) is located at a position corresponding to nucleotide 43 726 345 in Homo sapiens chromosome 13, GRCh37.p9 Primary Assembly (NCBI Reference Sequence: NC_{—}000013.10). SNP rs1156026 is located in a coding region from a yet uncharacterized gene, which spans nucleotides 43683486 to 43733602 of NCBI Reference Sequence NC_{—}000013.10 (corresponding to nucleotides 24663486 to 24713602 of NCBI Reference Sequence: NT_{—}024524.14). The sequence of this gene is depicted in FIGS. 13A-13K (SEQ ID NO: 9). This gene comprises about 50 kb of genomic DNA positioned between DNAJC15 and ENOX1, and comprises 5 putative exons making a total of about 5 kb of coding DNA. SNP rs1156026 is located in putative exon 3 of the gene, which spans nucleotides 43726126 to 43726400 of NCBI Reference Sequence NC_{—}000013.10. The sequence of rs1156026 provided in the NCBI SNP database is: CATAAGGACACTTTGAGGAAGACCCA[C/T]TTCCTGCAGGCAAGCAGGATGAAGT (SEQ ID NO: 1). This sequence is underlined in FIG. 13J. SNP rs1156026 was recently shown to be located in long intergenic non-protein coding RNA 400 (LINC00400).

In an embodiment, the psychiatric disorder is Schizophrenia, Schizophrenia-related disorders (e.g., SAD), bipolar disorder or Tourette's Syndrome. In an embodiment, the psychiatric disorder is major psychosis, such as schizophrenia (SZ), schizo-affective disorder (SAD) or bipolar disorder (BP). In a further embodiment, the psychiatric disorder is Schizophrenia or bipolar disorder. In yet a further embodiment, the onset of said Schizophrenia, schizo-affective disorder or bipolar disorder is at or before the age of about 26. In a further embodiment, the onset of said bipolar disorder is at or before the age of about 31.

In another aspect, the present invention provides a method for screening a subject for susceptibility or predisposition to BP, said method comprising determining the identity of one or more of insertions/deletions (indels) rs10569822, rs113886253 and rs146020684 alleles set forth in SEQ ID NOs: 12, 17 and 22, respectively, in a biological sample from said subject, and diagnosing said susceptibility or predisposition to BP based on the identity of said one or more indels.

Indels rs10569822, rs113886253 and rs146020684 are located in introns of the DnaJ (Hsp40) homolog, subfamily C, member 15 (DNAJC15) gene (Gene ID No. 29103, spanning positions 43023226 to 43109170 of Homo sapiens chromosome 13, GRCh38p2 Primary Assembly, NCBI Reference Sequence: NC_{—}000013.11).

Indel rs10569822 is located at a position corresponding to nucleotides 43098065 and 43098066 of chromosome 13 (intron 6 of the DNAJC15 gene), and the sequence provided in the NCBI SNP database is: TGGCCAAAGCAGTGGTTAGAGAAAA[-/AG]AAAATTTGCTATACTGAGAAACTTA (SEQ ID NO: 12).

Indel rs113886253 is located at a position corresponding to nucleotides 43036153 and 43036158 of chromosome 13 (intron 1 of the DNAJC15 gene), and the sequence provided in the NCBI SNP database is: TTAAATTGTGATTGGACAAAATGTT[-/TTTCAG]TTTCTGTCTTTTTTTTTTTTTTTTT (SEQ ID NO: 17).

Indel rs146020684 is located at a position corresponding to nucleotides 43051652 and 43051663 of chromosome 13 (intron 1 of the DNAJC15 gene), and the sequence provided in the NCBI SNP database is: ACTTCATGGTGTGTGTGTGTGTGTG[-/TGTGTGTGTGTA]TATATATCACATTTTCTTTATCCGC (SEQ ID NO: 22).

In another aspect, the present invention provides a method for the differential diagnosis of SZ and BP (or of a susceptibility/predisposition to SZ versus BP or vice versa) in a subject, said method comprising: (i) determining the identity of single nucleotide polymorphism (SNP) rs1156026 alleles set forth in SEQ ID NO: 1 in a biological sample (i.e. determining which allele(s) is/are present in the sample at the polymorphic nucleotide position of SNP rs1156026 (SEQ ID NO: 1)) from said subject); (ii) determining the identity of one or more of indels rs10569822, rs113886253 and/or rs146020684 alleles set forth in SEQ ID NOs: 12, 17 and 22, respectively, in said biological sample (i.e. determining which allele(s) is/are present in the sample at the polymorphic nucleotide positions of indels rs10569822, rs113886253 and/or rs146020684 (SEQ ID NOs: 12, 17 and 22)); wherein: (a) the presence of two cytosine (C)-containing alleles of SNP rs1156026 (CC genotype) and the absence of a deletion in rs10569822, rs113886253 and/or rs146020684 is indicative that said subject is more likely to suffer from BP, or has a susceptibility/predisposition thereto; (b) the presence of one or two thymine (T)-containing alleles of SNP rs1156026 (TC or TT genotype) and the absence of a deletion in rs10569822, rs113886253 and/or rs146020684 is indicative that said subject is more likely to suffer from SZ, or has a susceptibility/predisposition thereto. In an embodiment, the method comprises determining the identity of indels rs10569822, rs113886253 and rs146020684 in the sample.

The determination of the sequence of variants rs1156026, rs10569822, rs113886253 and/or rs146020684 in a biological sample may be performed by a number of methods which are known in the art (see, e.g., Syvanen, Nat Rev Genet. 2001 December; 2(12):930-42). Examples of suitable methods for determining sequences and polymorphisms at the nucleic acid level include sequencing of the nucleic acid sequence encompassing variants rs1156026, rs10569822, rs113886253 and/or rs146020684, e.g., in the genomic DNA; hybridization of a nucleic acid probe capable of specifically hybridizing to a nucleic acid sequence comprising one of the alleles and not to (or to a lesser extent to) a corresponding nucleic acid sequence comprising the other allele (under comparable hybridization conditions, such as stringent hybridization conditions) (e.g., molecular beacons); amplification of a nucleic acid fragment comprising the variants rs1156026, rs10569822, rs113886253 and/or rs146020684 using a primer specific for one of the allele (targeted allele), wherein the primer produces an amplified product if the targeted allele is present and does not produce the same amplified product when a nucleic acid sequence not comprising the targeted allele (comprising the other allele) is used as a template for amplification (e.g., allele-specific PCR), allele specific ligation, dynamic allele-specific hybridization (DASH) genotyping (Howell W., et al. (1999) Nat Biotechnol. 17(1):87-8), nucleic acid sequence based amplification (Nasba), primer extension assay, FLAP endonuclease assay (Invader assay, Olivier M. (2005). Mutat Res. 573(1-2):103-10), 5′ nuclease assay (McGuigan F. E. and Ralston S. H. (2002) Psychiatr Genet. 12(3):133-6), oligonucleotide ligase assay. Other methods include in situ hybridization analyses, single-stranded conformational polymorphism analyses, temperature gradient gel electrophoresis (TGGE), denaturing high performance liquid chromatography (DHPLC). Several SNP genotyping platforms are commercially available. Additional methods will be apparent to one of skill in the art.

In an embodiment, the present invention provides a method for diagnosing a psychiatric disorder (e.g., SZ) or a predisposition thereto in a subject, said method comprising sequencing a region of a nucleic acid encompassing SNP rs1156026 (SEQ ID NO: 1) in a sample from the subject to determine the identity of SNP rs1156026 alleles (i.e. to determine which allele(s) is/are present in the sample at the polymorphic nucleotide position of SNP rs1156026 (SEQ ID NO: 1)), and diagnosing said psychiatric disorder or predisposition thereto based on the identity of said SNP.

In an embodiment, the present invention provides a method for screening a subject for susceptibility or predisposition to BP, said method comprising sequencing a region of a nucleic acid encompassing rs10569822, rs113886253 and/or rs146020684 alleles set forth in SEQ ID NOs: 12, 17 and 22, respectively, in a biological sample from said subject to determine the identity of indels rs10569822, rs113886253 and/or rs146020684 alleles (i.e. to determine which allele(s) is/are present in the sample at the polymorphic nucleotide position of indels rs10569822, rs113886253 and/or rs146020684 (SEQ ID NOs: 12, 17 and 22)), and diagnosing said susceptibility or predisposition to BP based on the identity of said one or more indels. In an embodiment, the presence of a deletion allele of indels rs10569822, rs113886253 and/or rs146020684 (SEQ ID NOs: 15, 20 and 25) is indicative that the subject has a susceptibility or predisposition to BP.

In another aspect, the present invention provides a method for the differential diagnosis of SZ and BP (or of a susceptibility/predisposition to SZ versus BP or vice versa) in a subject, said method comprising:

(A) sequencing a region of a nucleic acid encompassing SNP rs1156026 (SEQ ID NO: 1) in a sample from the subject to determine the identity of SNP rs1156026 alleles (i.e. to determine which allele(s) is/are present in the sample at the polymorphic nucleotide position of SNP rs1156026 (SEQ ID NO: 1));
(B) sequencing a region of a nucleic acid encompassing rs10569822, rs113886253 and/or rs146020684 alleles set forth in SEQ ID NOs: 12, 17 and 22, respectively, in a biological sample from said subject to determine the identity of indels rs10569822, rs113886253 and/or rs146020684 alleles (i.e. to determine which allele(s) is/are present in the sample at the polymorphic nucleotide position of indels rs10569822, rs113886253 and/or rs146020684 (SEQ ID NOs: 12, 17 and 22)); wherein:
(a) the presence of two cytosine (C)-containing alleles of SNP rs1156026 (CC genotype) and the absence of a deletion in rs10569822, rs113886253 and/or rs146020684 is indicative that said subject is more likely to suffer from BP, or has a susceptibility/predisposition thereto; (b) the presence of one or two thymine (T)-containing alleles of SNP rs1156026 (TC or TT genotype) and the absence of a deletion in rs10569822, rs113886253 and/or rs146020684 is indicative that said subject is more likely to suffer from SZ, or has a susceptibility/predisposition thereto. In an embodiment, the method comprises sequencing and determining the identity of indels rs10569822, rs113886253 and rs146020684 in the sample.

The skilled person would understand that the determination of the identity of variants rs1156026, rs10569822, rs113886253 and/or rs146020684 may be performed “indirectly” by determining the identity (e.g., sequence) of the reverse complement of SEQ ID NOs: 1, 12, 17 and/or 22 in the biological sample, using for example any of the above-noted SNP genotyping methods.

All these detection techniques may also be employed in the format of microarrays (e.g., SNP microarrays), antibody microarrays (e.g., using anti-DNA antibodies capable of specifically binding to one of the allele), tissue microarrays, electronic biochip (see Schena M., Microarray Biochip Technology, Eaton Publishing, Natick, Mass., 2000).

Further, nucleic acid-containing sequences may be amplified prior to or in conjunction with the detection methods noted herein. The design of various primers for such amplification is known in the art. For example, a nucleic acid (e.g., genomic DNA) comprising the variant(s) may be amplified using primers hybridizing to sequences upstream and downstream of variants rs1156026 (see FIGS. 13A-13K, particularly FIG. 13J), rs10569822, rs113886253 and/or rs146020684, to amplify a nucleic acid encompassing variants rs1156026, rs10569822, rs113886253 and/or rs146020684 (a nucleic acid of any length, for example from about 100 to 1000 nucleotides). Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the 013 replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). In an embodiment, amplification is carried out using PCR.

Polymerase chain reaction (PCR) may be carried out in accordance with known techniques. See, e.g., U.S. Pat. Nos. 4,683,195; 4,683,202; 4,800,159; and 4,965,188. In general, PCR involves a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out for example by visualization following Ethidium Bromide (EtBr) staining of the DNA following gel electrophoresis, or using a detectable label in accordance with known techniques, and the like. For a review on PCR techniques (see PCR Protocols, A Guide to Methods and Amplifications, Michael et al. Eds, Acad. Press, 1990). The identity of the SNP may be detected using a detectable (labeled) probe, for example.

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel, et al. (eds), 1989, Current Protocols in Molecular Biology, Vol. 1, Green Publishing Associates, Inc., and John Wiley & Sons, Inc., New York) and are commonly known in the art. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (see Ausubel, et al. (eds), 1989, supra). In other examples of hybridization, a nitrocellulose filter can be incubated overnight at 65° C. with a labeled probe specific to one or the other two alleles in a solution containing 50% formamide, high salt (5×SSC or 5×SSPE), 5×Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York). The selected temperature is based on the melting temperature (Tm) of the DNA hybrid (Sambrook et al. 1989, supra). Generally, stringent conditions are selected to be about 5° C. lower than the thermal melting point for the specific sequence at a defined ionic strength and pH.

In an embodiment, the present invention also provides a method for diagnosing a psychiatric disorder or a predisposition thereto in a subject, said method comprising (i) amplifying a region of a nucleic acid encompassing SNP rs1156026 (SEQ ID NO: 1) in a sample from the subject, (ii) determining the identity of SNP rs1156026 alleles in the sample comprising the amplified product (i.e. to determine which allele(s) is/are present in the sample comprising the amplified product at the polymorphic nucleotide position of SNP rs1156026 (SEQ ID NO: 1)), and (iii) diagnosing said psychiatric disorder or predisposition thereto based on the identity of said SNP. In an embodiment, the amplifying comprises contacting the nucleic acid with a set of primers, and under conditions, allowing amplification of the region encompassing SNP rs1156026 (SEQ ID NO: 1). An example of a pair of primers suitable to amplify a region encompassing SNP rs1156026 is 5′-GCCCTGCCTAATGCACTTTCTGATG-3′ (SEQ ID NO: 10, forward primer) and 5′-CTTTTATAATCCAAATTATTATGGC-3′ (SEQ ID NO: 11, reverse primer), which generates an amplified product of about 170-180 (e.g., 176) base pairs. The sequences corresponding to SEQ ID NO: 10, and to the reverse complement of SEQ ID NO: 11, are italicized in FIG. 13J.

In an embodiment, the present invention also provides a method for screening a subject for susceptibility or predisposition to BP, said method comprising (i) amplifying one or more regions of a nucleic acid encompassing one or more of indels rs10569822, rs113886253 and/or rs146020684 (SEQ ID NOs: 12, 17 and/or 22) in a sample from the subject, (ii) determining the identity of indels rs10569822, rs113886253 and/or rs146020684 alleles in the sample comprising the amplified product(s) (i.e. to determine which allele(s) is/are present in the sample comprising the amplified product(s) at the polymorphic nucleotide positions of indels rs10569822, rs113886253 and/or rs146020684), and (iii) determining the subject's susceptibility or predisposition to BP based on the identity of said one or more indels. In an embodiment, the amplifying comprises contacting the nucleic acid with one or more sets of primers, and under conditions, allowing amplification of the region(s) encompassing indels rs10569822, rs113886253 and/or rs146020684.

An example of a pair of primers suitable to amplify a region encompassing indel rs10569822 is 5′-TGAGGAAAGATCACTCCTGG-3′ (SEQ ID NO: 27, forward primer) and 5′-CTGTCCTCATAGAGGCATAC-3′ (SEQ ID NO: 28, reverse primer), which generates an amplified product of about 172-174 base pairs. The deletion and native reference sequences correspond to PCR fragments of 172 and 174 base pairs, respectively.

An example of a pair of primers suitable to amplify a region encompassing indel rs113886253 is 5′-CTGTGCCCAGTCCAAAGTAC-3′ (SEQ ID NO: 29, forward primer) and 5′-AGCCTGGATGACAGTGAGAC-3′ (SEQ ID NO: 30, reverse primer), which generates an amplified product of about 209-217 base pairs. The deletion and native reference sequences correspond to PCR fragments of 211 and 217 base pairs, respectively.

An example of a pair of primers suitable to amplify a region encompassing indel rs146020684 is 5′-GCTGCAAATGCCGTTATTTG-3′ (SEQ ID NO: 31, forward primer) and 5′-TCTTGGGTATCCAACCAGAG-3′ (SEQ ID NO: 32, reverse primer), which generates an amplified product of about 232-250 base pairs. The main deletion and native reference sequences correspond to PCR fragments 234 and 246 base pairs, respectively, and one additional rarer deletion generating a PCR fragment of 232, as well as two rare insertions generating PCR fragments of 248 and 250, were also detected.

In another aspect, the present invention provides a method for the differential diagnosis of SZ and BP (or of a susceptibility/predisposition to SZ versus BP or vice versa) in a subject, said method comprising:

(A) (i) amplifying a region of a nucleic acid encompassing SNP rs1156026 (SEQ ID NO: 1) in a sample from the subject, (ii) determining the identity of SNP rs1156026 alleles in the sample comprising the amplified product (i.e. to determine which allele(s) is/are present in the sample comprising the amplified product at the polymorphic nucleotide position of SNP rs1156026 (SEQ ID NO: 1)),
(B) (i) amplifying one or more regions of a nucleic acid encompassing one or more of indels rs10569822, rs113886253 and/or rs146020684 (SEQ ID NOs: 12, 17 and/or 22) in a sample from the subject, (ii) determining the identity of indels rs10569822, rs113886253 and/or rs146020684 alleles in the sample comprising the amplified product(s) (i.e. to determine which allele(s) is/are present in the sample comprising the amplified product(s) at the polymorphic nucleotide positions of indels rs10569822, rs113886253 and/or rs146020684); wherein:
(a) the presence of two cytosine (C)-containing alleles of SNP rs1156026 (CC genotype) and the absence of a deletion in rs10569822, rs113886253 and/or rs146020684 is indicative that said subject is more likely to suffer from BP, or has a susceptibility/predisposition thereto; (b) the presence of one or two thymine (T)-containing alleles of SNP rs1156026 (TC or TT genotype) and the absence of a deletion in rs10569822, rs113886253 and/or rs146020684 is indicative that said subject is more likely to suffer from SZ, or has a susceptibility/predisposition thereto. In an embodiment, the method comprises sequencing and determining the identity of indels rs10569822, rs113886253 and rs146020684 in the sample.

In an embodiment, the present invention also provides a method for diagnosing a psychiatric disorder or a predisposition thereto in a subject, said method comprising (i) amplifying a region of a nucleic acid encompassing SNP rs1156026 (SEQ ID NO: 1) in a sample from the subject, (ii) (ii) sequencing the amplified product, or a portion thereof encompassing SNP rs1156026 (SEQ ID NO: 1) in a sample from the subject to determine the identity of SNP rs1156026 alleles (i.e. to determine which allele(s) is/are present in the sample at the polymorphic nucleotide position of SNP rs1156026 (SEQ ID NO: 1)), and (iii) diagnosing said psychiatric disorder or predisposition thereto based on the identity of said SNP. In an embodiment, the amplifying comprises contacting the nucleic acid with a set of primers, and under conditions, allowing amplification of the region encompassing SNP rs1156026 (SEQ ID NO: 1).

In an embodiment, the present invention also provides a method for screening a subject for susceptibility or predisposition to BP, said method comprising (i) amplifying a region of a nucleic acid encompassing one or more of indels rs10569822, rs113886253 and/or rs146020684 (SEQ ID NOs: 12, 17 and/or 22) in a sample from the subject, (ii) sequencing the amplified product, or a portion thereof encompassing one or more of indels rs10569822, rs113886253 and/or rs146020684 to determine the identity of the indels rs10569822, rs113886253 and/or rs146020684 alleles in the sample (i.e. to determine which allele(s) is/are present in the sample comprising the amplified product at the polymorphic nucleotide positions of indels rs10569822, rs113886253 and/or rs146020684), and (iii) determining the subject's susceptibility or predisposition to BP based on the identity of said one or more indels. In an embodiment, the amplifying comprises contacting the nucleic acid with one or more sets of primers, and under conditions, allowing amplification of the region(s) encompassing indels rs10569822, rs113886253 and/or rs146020684.

In an embodiment, the present invention also provides a method for diagnosing a psychiatric disorder or a predisposition thereto in a subject, said method comprising (i) contacting a nucleic acid in a sample from the subject with a probe capable of specifically hybridizing to one of the alleles of SNP rs1156026, (ii) determining which allele(s) is/are present in the sample based on the presence or absence of hybridization between the probe and the nucleic acid, and (iii) diagnosing said psychiatric disorder or predisposition thereto based on the identity of the allele(s) present in the sample. In an embodiment, the method further comprises amplifying a region of a nucleic acid encompassing SNP rs1156026 (SEQ ID NO: 1) in a sample from the subject, and contacting the amplified product with the probe. In a further embodiment, the amplifying comprises contacting the nucleic acid with a set of primers, and under conditions, allowing amplification of the region encompassing SNP rs1156026 (SEQ ID NO: 1).

In an embodiment, the present invention also provides a method for screening a subject for susceptibility or predisposition to BP, said method comprising (i) contacting a nucleic acid in a sample from the subject with one or more probe(s) capable of specifically hybridizing to one of the alleles of one or more of indels rs10569822, rs113886253 and/or rs146020684, (ii) determining which allele(s) is/are present in the sample based on the presence or absence of hybridization between the probe(s) and the nucleic acid, and (iii) determining the subject's susceptibility or predisposition to BP based on the identity of the allele(s) present in the sample. In an embodiment, the method further comprises amplifying one or more regions of a nucleic acid encompassing indels rs10569822, rs113886253 and/or rs146020684 (SEQ ID NOs: 12, 17 and/or 22) in a sample from the subject, and contacting the amplified product(s) with the probe(s). In a further embodiment, the amplifying comprises contacting the nucleic acid with one or more sets of primers, and under conditions, allowing amplification of the region(s) encompassing indels rs10569822, rs113886253 and/or rs146020684 (SEQ ID NOs: 12, 17 and/or 22).

In embodiments, one or more of the methods described herein are performed in vitro.

In an embodiment, the above-noted method further comprises selecting a subject suspected of suffering from a psychiatric disorder, or suspected of being predisposed to developing a psychiatric disorder (e.g., based on family antecedents and/or other risk factors, for example).

In another embodiment, the above-noted method further comprises obtaining or collecting a biological sample from a subject. In various embodiments, the above-noted sample can be from any source that contains biological material suitable for the detection of the SNP, such as genomic DNA, for example a tissue or body fluid from the subject (blood, immune cells (e.g., lymphocytes), epithelia, a neural cell sample, etc. The sample may be subjected to commonly used isolation and/or purification techniques for enrichment in nucleic acids (e.g., genomic DNA). Accordingly, in an embodiment, the method may be performed on an isolated nucleic acid sample, such as isolated genomic DNA. The biological sample may be collected using any methods for collection of a biological fluid, tissue or cell sample, such as venous puncture for collection of blood-derived samples (e.g., whole blood, peripheral blood mononuclear cells (PBMCs), etc.).

In an embodiment, the above-mentioned method is an aid for the diagnosis of psychiatric disorders (e.g., SZ, BP). Accordingly, the above-mentioned methods may be performed in combination with other assays, methods or markers for diagnosing psychiatric disorders, for example evaluation by a trained mental-health professional, administration of a variety of personality tests and neuropsychological tests, neurocognitive measurements, gathering of background (including medical) information about the individual (e.g., patient's self-reported experiences, behavior reported by relatives or friends), presence of biological and/or other genetic markers associated with the psychiatric disorder, electroretinographic (ERG) measurements (see, U.S. provisional application No. 61/781,520 and WO 2014/138987), etc. For example, the above-mentioned method may be combined with the criteria set forth in the Diagnostic and Statistical Manual of Mental Disorders 5^{th} Edition (DSM-V), or the World Health Organization's International Classification of Diseases and Related Health Problems (ICD-10), or with any other biological markers (e.g., other SNPs or blood biomarkers) known to be associated with a psychiatric disorder or a predisposition thereto (see, e.g., Chan et al., Int Rev Neurobiol. 2011; 101:95-144). In an embodiment, the disorder is SZ, a SZ-related disorder or bipolar disorder and the method further comprises measuring an episodic memory parameter in the subject. In a further embodiment, the episodic memory parameter is verbal episodic memory (VEM), visual episodic memory (VisEM), or both.

In an embodiment, the disorder is TS and the method further comprises determining the sequence of one or more of SNPs rs1323142 (SEQ ID NO: 6), rs883877 (SEQ ID NO: 7) and rs9567343 (SEQ ID NO: 8) in a biological sample from said subject. In a further embodiment, the method further comprises determining the sequence of SNP rs9567343 (SEQ ID NO: 8) in a biological sample from the subject, in yet a further embodiment the method comprises determining the sequence of SNPs rs1323142 (SEQ ID NO: 6), rs883877 (SEQ ID NO: 7) and rs9567343 (SEQ ID NO: 8) in a biological sample from the subject.

The invention further provides an oligonucleotide (e.g., a probe or a primer), capable of specifically hybridizing to a nucleotide sequence corresponding to one of the alleles and not to (or to a lesser extent to) a nucleic acid sequence corresponding to the other allele (under comparable hybridization conditions). Such hybridization may be under moderately stringent, or preferably stringent, conditions, as noted herein. Such an oligonucleotide may in embodiments be attached to a solid substrate, as noted herein. Such oligonucleotides may be used to specifically detect the presence of a nucleic acid (genomic DNA, cDNA) corresponding to one of the alleles in a sample. In an embodiment, such an oligonucleotide hybridizes to a portion of the nucleic acid of SEQ ID NO: 1 encompassing the SNP, or to its complement. In another embodiment, such an oligonucleotide hybridizes to a portion of the nucleic acid of SEQ ID NOs: 12, 17 or 22 encompassing the indel, or to its complement.

In an embodiment, the oligonucleotide specifically hybridizes to the following nucleotide sequence or a portion thereof encompassing the T underlined therein CATAAGGACACTTTGAGGAAGACCCATTTCCTGCAGGCAAGCAGGATGAAGT (SEQ ID NO: 2), or to the complement thereof (ACTTCATCCTGCTTGCCTGCAGGAAATGGGTCTTCCTCAAAGTGTCCTTATG, SEQ ID NO: 3), but not to (or to a lesser extent to) a corresponding sequence or portion thereof wherein the T underlined is replaced with a C, or to the complement thereof.

In another embodiment, the oligonucleotide specifically hybridizes to the following nucleotide sequence or a portion thereof encompassing the C underlined therein CATAAGGACACTTTGAGGAAGACCCACTTCCTGCAGGCAAGCAGGATGAAGT (SEQ ID NO: 4), or to the complement thereof (ACTTCATCCTGCTTGCCTGCAGGAAGTGGGTCTTCCTCAAAGTGTCCTTATG, SEQ ID NO: 5), but not to (or to a lesser extent to) a corresponding sequence or portion thereof wherein the C underlined is replaced with a T (e.g., SEQ ID NO: 2), or to the complement thereof (e.g., SEQ ID NO: 3).

In an embodiment, the oligonucleotide specifically hybridizes to the following nucleotide sequence or a portion thereof encompassing the AG underlined therein TGGCCAAAGCAGTGGTTAGAGAAAAAGAAAATTTGCTATACTGAGAAACTTA (SEQ ID NO: 13) or to the complement thereof TAAGTTTCTCAGTATAGCAAATTTTCTTTTTCTCTAACCACTGCTTTGGCCA (SEQ ID NO: 14), but not to (or to a lesser extent to) a corresponding sequence or portion thereof wherein the AG underlined is absent (SEQ ID NO: 15), or to the complement thereof (SEQ ID NO: 16).

In an embodiment, the oligonucleotide specifically hybridizes to the following nucleotide sequence or a portion thereof TGGCCAAAGCAGTGGTTAG--AAAAAGAAAATTTGCTATACTGAGAAACTTA (SEQ ID NO: 13) or to the complement thereof TAAGTTTCTCAGTATAGCAAATTT--TTTTTCTCTAACCACTGCTTTGGCCA (SEQ ID NO: 14), but not to (or to a lesser extent to) a corresponding sequence or portion thereof wherein the - are replaced by AG (SEQ ID NO: 13), or to the complement thereof (SEQ ID NO: 14).

In an embodiment, the oligonucleotide specifically hybridizes to the following nucleotide sequence or a portion thereof encompassing the TTTCAG underlined therein: TTAAATTGTG ATTGGACAAAATGTTTTTCAGTTTCTGTCTTTTTTTTTTTTTTTTT (SEQ ID NO: 18) or to the complement thereof AAAAAAAAAAAAAAAAAGACAGAAACTGAAAAACATTTTGTCCAATCACAATTTAA (SEQ ID NO: 19), but not to (or to a lesser extent to) a corresponding sequence or portion thereof wherein the TTTCAG underlined is absent (SEQ ID NO: 20), or to the complement thereof (SEQ ID NO: 21).

In an embodiment, the oligonucleotide specifically hybridizes to the following nucleotide sequence or a portion thereof TTAAATTGTGATTGGACAAAATGTT------ TTTCTGTCTTTTTTTTTTTTTTTTT (SEQ ID NO: 20) or to the complement thereof AAAAAAAAAAAAAAAAAGACAGAAA------AACATTTTGTCCAATCACAATTTAA (SEQ ID NO: 21), but not to (or to a lesser extent to) a corresponding sequence or portion thereof wherein the ------ are replaced by TTTCAG (SEQ ID NO: 18), or to the complement thereof (SEQ ID NO: 19).

In an embodiment, the oligonucleotide specifically hybridizes to the following nucleotide sequence or a portion thereof encompassing the TGTGTGTGTGTA underlined therein:

(SEQ ID NO: 23)
ACTTCATGGTGTGTGTGTGTGTGTGTGTGTGTGTGTATATATATCACATT
TTCTTTATCCGC

or to the complement thereof

(SEQ ID NO: 24)
GCGGATAAAGAAAATGTGATATATATACACACACACACACACACACACAC
ACACCATGAAGT,

but not to (or to a lesser extent to) a corresponding sequence or portion thereof wherein the TGTGTGTGTGTA underlined is absent (SEQ ID NO: 25), or to the complement thereof (SEQ ID NO: 26).

In an embodiment, the oligonucleotide specifically hybridizes to the following nucleotide sequence or a portion thereof ACTTCATGGTGTGTGTGTGTGTGTG------------TATATATCACATTTTCTTTATCCGC (SEQ ID NO: 25) or to the complement thereof GCGGATAAAGAAAATGTGATATATA------------CACACACACACACACACCATGAAGT (SEQ ID NO: 26), but not to (or to a lesser extent to) a corresponding sequence or portion thereof wherein the ------------ are replaced by TGTGTGTGTGTA (SEQ ID NO: 23), or to the complement thereof (SEQ ID NO: 24).

Oligonucleotide probes or primers of the present invention may be of any suitable length, depending on the particular assay format and the particular needs and targeted sequences employed. In general, the oligonucleotide probes or primers are at least about 6 nucleotides in length, in an embodiment at least about 12 nucleotides in length, preferably from about 12 to about 100 nucleotides in length, in embodiments from about 12 to about 50, from about 12 to about 30, or from about 15 to about 24 nucleotides in length. They may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2^{nd} Edition, CSH Laboratories; Ausubel et al., 1989, in Current Protocols in Molecular Biology, John Wiley & Sons Inc., N.Y.).

Probes or primers of the invention can be utilized with naturally-occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes or primers of the invention can be constructed of either ribonucleic acid (RNA) or deoxyribonucleic acid (DNA), and preferably of DNA.

Although the present invention is not specifically dependent on the use of a label for the detection of a particular nucleic acid sequence, such a label might be beneficial for the detection of SNPs. Furthermore, it enables automation. Probes can be labeled according to numerous well-known methods (Sambrook et al., 1989, supra). Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will be understood by the person of ordinary skill that the choice of a particular label dictates the manner in which it is bound to the probe.

As commonly known, radioactive nucleotides can be incorporated into probes by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g., uniformly labeled DNA probe using random oligonucleotide primers in low-melt gels), using the SP6/T7 system to transcribe a DNA segment in the presence of one or more radioactive NTP, and the like.

In an embodiment, the method comprises contacting a sample from the subject, the sample comprising a nucleic acid (genomic DNA), with one or more oligonucleotides (probes and/or primers) as defined herein to determine the identity of single nucleotide polymorphism (SNP) rs1156026 (set forth in SEQ ID NO: 1), and/or insertion/deletion (indel) rs10569822 (set forth in SEQ ID NO: 12), rs113886253 (set forth in SEQ ID NO: 17) and/or rs146020684 (set forth in SEQ ID NO: 22).

The present invention also relates to a kit for diagnosing a psychiatric disorder or a predisposition thereto, or for determining the risk that a subject develops schizophrenia (SZ), schizo-affective disorder (SAD) or early onset (e.g., before the age of about 26) SZ, SAD or BP, the kit comprising one or more suitable reagents to detect a nucleic acid comprising SNP rs1156026, such as a probe, primer (or primer pair).

In another aspect, the present invention provides a kit for screening a subject for susceptibility or predisposition to BP, the kit comprising (i) at least one reagent such as a probe, primer (or primer pair) as defined herein for determining the identity of one or more of insertion/deletion (indel) rs10569822, rs113886253 and/or rs146020684 alleles set forth in SEQ ID NOs: 12, 17 and 22, respectively, in a biological sample from said subject.

In another aspect, the present invention provides a kit for the differential diagnosis of SZ and BP (or of a susceptibility/predisposition to SZ versus BP or vice versa) in a subject, said kit comprising: (i) at least one reagent such as a probe, primer (or primer pair) as defined herein for determining the identity of single nucleotide polymorphism (SNP) rs1156026 set forth in SEQ ID NO: 1 in a biological sample from the subject; and (ii) at least one reagent for determining the identity of one or more of insertion/deletion (indel) rs10569822, rs113886253 and/or rs146020684 alleles set forth in SEQ ID NOs: 12, 17 and 22, respectively, in a biological sample from said subject.

For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers may for example include a container which will accept the test sample (e.g., DNA and/or cells), a container which contains the primers used in the assay, containers which contain enzymes, containers which contain wash reagents, and containers which contain the reagents used to perform the method and/or detect the indicator products (buffers, solutions, enzymes, etc.). In an embodiment, the kit further comprises instructions for diagnosing a psychiatric disorder or a predisposition thereto, or for determining the risk that a subject develops early onset schizophrenia (SZ), schizo-affective disorder (SAD) or bipolar disorder (BP), using the above-mentioned methods.

In another aspect, the present invention provides an assay mixture comprising: (i) a biological sample comprising nucleic acids from a subject, e.g., a subject suspected or at risk of suffering from a psychiatric disorder); and (ii) at least one reagent such as a probe, primer (or primer pair) as defined herein for determining the identity of single nucleotide polymorphism (SNP) rs1156026 set forth in SEQ ID NO: 1.

In another aspect, the present invention provides an assay mixture comprising: (i) a biological sample comprising nucleic acids from a subject, e.g., a subject suspected or at risk of suffering from a psychiatric disorder; and (ii) at least one reagent such as a probe, primer (or primer pair) as defined herein for determining the identity of one or more of insertion/deletion (indel) rs10569822, rs113886253 and/or rs146020684 alleles set forth in SEQ ID NOs: 12, 17 and 22, respectively.

In another aspect, the present invention provides an assay mixture comprising: (i) a biological sample comprising nucleic acids from a subject, e.g., a subject suspected or at risk of suffering from a psychiatric disorder (e.g., suspected or at risk of suffering from SZ or BP); (ii) at least one reagent such as a probe, primer (or primer pair) as defined herein for determining the identity of single nucleotide polymorphism (SNP) rs1156026 set forth in SEQ ID NO: 1; and (iii) at least one reagent such as a probe, primer (or primer pair) as defined herein for determining the identity of one or more of insertion/deletion (indel) rs10569822, rs113886253 and/or rs146020684 alleles set forth in SEQ ID NOs: 12, 17 and 22, respectively.

In an embodiment, the assay mixture further comprises one or more reagents to perform the assay (reagents to perform nucleic acid amplification, hybridization and/or detection), for example suitable buffers, solutions and/or enzymes.

In certain embodiments, the methods of the present invention further comprise sending the diagnostic results (i.e., whether or not the subject has a psychiatric disorder or a predisposition thereto) to a clinician, e.g., a psychiatrist or a general practitioner.

In an embodiment, the above-mentioned methods further comprise selecting and/or administering a course of therapy or prophylaxis to said subject in accordance with the diagnostic result.

Accordingly, in another aspect, the present invention provides a method for preventing or treating a psychiatric disorder, said method comprising:
identifying a subject suffering from a psychiatric disorder or a predisposition thereto using a method defined above; and
administering a course of therapy or prophylaxis to said subject.

The term “course of therapy” or “therapy” includes any therapeutic approach taken to relieve and/or prevent one or more symptoms associated with a psychiatric disorder. The term encompasses administering any compound, drug, therapeutic agent, procedure, or regimen useful for improving the health of a subject with a psychiatric disorder. In an embodiment, the course of therapy or prophylaxis comprises administration of a psychotropic medication. Psychotropic medication as used herein refers to drugs used for the management of mental and emotional disorders such as psychiatric disorders, and includes for example antidepressants, stimulants, antipsychotics, mood stabilizers, anxiolytics. In an embodiment, the psychotropic medication comprises an antipsychotic medication. In an embodiment, the method comprises identifying a subject suffering from SZ or having a predisposition thereto using the methods defined herein, and administering a suitable therapy for SZ to said subject. In another embodiment, the method comprises identifying a subject suffering from BP or having a predisposition thereto using the methods defined herein, and administering a suitable therapy for BP to said subject.

In an embodiment, one or more steps of the above-mentioned methods are performed using or by a computer (e.g., using computer/computing algorithms), using a suitably programmed computer. According to various embodiments, the method can further comprise determining the identity of SNP rs1156026 set forth in SEQ ID NO: 1, indel rs10569822 (set forth in SEQ ID NO: 12), indel rs113886253 (set forth in SEQ ID NO: 17) and/or indel rs146020684 (set forth in SEQ ID NO: 22) in a subject. In an embodiment, the data obtained can subsequently be stored in a computer in a suitable computer readable form. The computer can subsequently be used to analyze the data and compare them to a control, determine an algorithm, apply the algorithm, etc. The data or results can then be displayed, for example, on a monitor, and/or printed. In embodiments, the methods further comprise transmitting the data or results over a communication network. For example, the data or results may be transferred from a laboratory testing facility (e.g., diagnostic laboratory) to a health care provider, who may analyse the data/results and/or choose the appropriate course of action based on the data/results (e.g., initiate therapy, continue therapy, interrupt therapy, modify the therapy, etc.).

In another embodiment, the methods of the present invention can include processing or converting the raw target detection data (e.g., mathematically, statistically or otherwise) using a statistical method (e.g., logistic or logit regression, cluster analysis, ANCOVA) that takes into account subject data or other data. Subject data may include (but is not limited to): age; race; disease stage/phase, medication, family history, etc. The algorithm may also take into account factors such as the presence, diagnosis and/or prognosis of a subject's condition other than the major psychiatric disorder. As will be clear to the skilled artisan to which the present invention pertains, from above and below, numerous combinations of data parameters and/or factors may be used by the algorithm or algorithms encompassed herein, to obtain the desired output.

As used herein, the term “subject” means an individual. In an embodiment, the subject is a human. As used herein, a “subject” is the same as a “patient,” and the terms can be used interchangeably. In an embodiment, the subject is suspected of suffering from, or of having a predisposition to, the psychiatric disorder.

### MODE(S) FOR CARRYING OUT THE INVENTION

The present invention is illustrated in further details by the following non-limiting examples.

### Example 1

### Methods

Study Subjects and Phenotype Definition.

The case-control sample consisted of 247 unrelated SZ cases and 250 unrelated normal controls without any psychiatric diagnosis from the Eastern Quebec population. All subjects were Caucasian of French-Canadian ancestry. The proportion of males was 79 percent among the cases and 78 percent among the controls. Controls were adults, with a median age of 45 at the time of psychiatric evaluation. Among cases, the median age of onset of SZ was 24 and the interquartile range 20-29.5. A lifetime best-estimate DSM-IV diagnosis was made for the unrelated SZ cases and the kindred members using personal interview, information from relatives and extensive medical records. A stringent diagnosis procedure outlined in previous reports (Maziade, M., et al., Am J Psychiatry, 1992. 149(12): p. 1674-86; Roy, M. A., et al., Am J Psychiatry, 1997. 154(12): p. 1726-33) was applied. In brief, all available information across lifetime from different sources (all medical records, family informant interviews, personal structured interview) was reviewed blindly by four research diagnosticians. The board of diagnosticians also specified the presence or absence of psychotic features in BP patients according to DSM-IV. Family history of mental disorders of the SZ unrelated cases was also obtained from the same sources. The kindred sample consisted of 48 multigenerational families of which 21 were mainly affected by BP (<15% of the affected family members had SZ), 15 mainly affected by a SZ spectrum disorder (<15% had BP) and 12 were mixed pedigrees, i.e. affected almost equally by SZ and BP. In the kindred sample, a narrow SZ definition restricted to SZ and a broad definition comprising SZ narrow plus schizophreniform disorder and schizotypal personality was used as phenotype. The BP narrow phenotype was restricted to BP I, and the broad definition included BP I, BP II, and recurrent major depression. A narrow and broad “common locus” (CL) phenotype was also defined. The narrow CL phenotype included BP narrow, SZ narrow and schizoaffective disorder (SAD). The broad CL definition included the broad definitions of BP and SZ, in addition to SAD. The number of affected subjects for each phenotype definition is reported in Table 1. A comparison group for the association analyses with the 467 genotyped non affected subjects from the family sample satisfying the following criteria was formed: A) no diagnosis in the broad definition of CL, B) age greater or equal to 25 years and C) not a parent of a CL case. These subjects are referred to as non-affected adult relatives (NAARs). In total, 845 subjects were included in the association analyses.

TABLE 1
Number of affected subjects for each phenotype definition.
Phenotypes

| | | |
|---|---|---|
| CL | SZ | BP |

| narrow | broad | narrow | broad | narrow | broad |
|---|---|---|---|---|---|
| 281 | 378 | 125 | 136 | 120 | 205 |

Abbreviations: SZ, schizophrenia; BP, bipolar; CL refers to the “common locus” phenotype as defined above.

Candidate Region Definition.

A wide region was encompassed, given the uncertainty in the boundaries of linkage signals for complex traits. The two markers delimiting the linkage region with a −log₁₀ (p-value) above 3.0 in the kindred sample, D1351491 and D1351272, were taken as anchor points (Maziade, M., et al., Eur J Hum Genet, 2009. 17(8): p. 1034-42), and the candidate region was defined as extending 5 Mb proximal from D1351491 to 5 Mb distal from D1351272. The interval corresponds to coordinates 33.564 to 50.086 Mb in human genome assembly build GRCH37.3.

Genotyping and CNV Detection.

In the case-control sample, SNPs were analyzed using the mini-sequencing approach of the Illumina™ genotyping platform with a customized array including the HumanHap300 BeadChip™ and 57,000 additional SNPs, for a total of 375,174 SNPs. All subjects had a genotype called for at least 97 percent of the SNPs. The region of interest derived from the linkage evidence in our kindred sample contained 2,150 SNPs. For replication in the kindred sample, SNPs were analyzed using an in house minisequencing approach (Sun, X., et al., Nucleic Acids Research, 2000. 28(12): p. e68) adapted for the LiCor™ sequencers, where genotypes are called automatically using the software SAGA™ (LICOR). A melting temperature procedure with a cold oligonucleotide probe specific to one of the two nucleotides of the SNP was also used with High Resolution™ Melting kit and a real time PCR (480 LightCycler™), both from Roche. Mendelian inheritance was checked using the computer software PedCheck™ (O'Connell, J. R. and D. E. Weeks, American Journal of Human Genetics, 1998. 63: p. 259-266), and 10% blind replicates were included for genotyping quality control. Copy number variants (CNVs) were inferred using the hidden Markov model implemented in PennCNV™ (Wang, K., et al., Genome Res, 2007. 17(11): p. 1665-74), which uses the log R ratio and B allele frequency produced by the Illumina BeadStudio™ software to infer hidden states corresponding to copy number. Population frequencies of the B allele were estimated from our sample and other model parameters were set to the values estimated by Wang et al. (2007, supra).

Genotyping Quality Criteria in the Case-Control Sample.

Only SNPs with a minimum call rate of 98 percent and minor allele frequency above one percent in the combined case-control sample were retained. SNPs with Hardy-Weinberg equilibrium chi-square test p-value less than 2.5×10⁻⁵, corresponding to a 0.05 significance level divided by the number of SNPs in the region, were discarded. This left 2,081 SNPs to be included in the analysis. Only 2 discordant genotypes were observed at these 2,081 SNPs among 30 subjects genotyped in duplicate, for a concordance rate of 99.997 percent.

Assessment of Population Substructure.

A principal component (PC) analysis of the genotypes of the 339,228 autosomal SNPs was performed with Eigensoft™ version 3.0 (Patterson, N., A. L. Price, and D. Reich, PLoS Genet, 2006. 2(12): p. e190, genepath.med.harvard.eduhreich/Software.htm) to investigate population substructure in our case-control sample. Ancestry differences between the case and control samples were tested by comparing the two groups on the first ten PCs using a Tracy-Widom statistic implemented in Eigensoft. The software was also used to estimate the inflation factor λ based on the genomic control method (Devlin, B., K. Roeder, and L. Wasserman, Genomic control, a new approach to genetic-based association studies. Theor Popul Biol, 2001. 60(3): p. 155-66).

Association Analysis in the Case-Control Sample.

Standard allelic, trend and genotypic tests under a dominant and an additive model were performed and allelic and genotypic odds ratios were estimated on the genotyped SNPs and on untyped SNPs imputed using genotype data from the 1000 Genomes Project. Association to both genotyped and imputed SNPs was also tested conditionally on genotypes and on allele counts of the genotyped SNPs showing the strongest association. Haplotype association tests were performed on sliding windows of three and five consecutive SNPs.

More specifically, allelic association with genotyped SNPs was tested using Fisher exact tests in the 2×2 table of alleles x case-control status, as well as Cochran-Armitage trend tests in the 3×2 table of genotype x case-control status. Fisher exact tests were also performed with genotype frequencies under the dominant and recessive models. We tested association with untyped variants in the region extending 200 kb on either side of the association signal detected using genotyped SNPs in an attempt to better characterize the association. Two complementary approaches were applied: imputation of genotypes at untyped SNPs and global tests of haplotypes (frequency >1%) over short windows of SNPs (Huang, B. E., C. I. Amos, and D. Y. Lin, Genet Epidemiol, 2007. 31(8): p. 803-12). Genotype imputation was performed using genotype data from the 1000 Genomes Project (June 2011 data release, www.1000genomes.org, The 1000 Genomes Project Consortium, Nature 2010 vol. 467: 1061-73) using IMPUTE™ version 2 (Howie, B. N., P. Donnelly, and J. Marchini PLoS Genet, 2009. 5(6): p. e1000529, mathgen.stats.ox.ac.uk/impute/impute.html). Association with the imputed SNP genotypes was then tested using a score test derived from the missing data likelihood under a logistic model implemented in SNPTEST™ version 2.2 (mathgen.stats.ox.ac.uk/genetics_software/snptest/snptest.html; J. Marchini, et al. (2007). Nature Genetics 39: 906-913). Association to both genotyped and imputed SNPs was also tested conditionally on genotypes and on allele counts of the genotyped SNPs showing the strongest association. We used the score test under a logistic model implemented in SNPTEST™. Haplotype association tests were performed on sliding windows of three and five consecutive SNPs. Association to any haplotype formed by the SNPs in each window and to individual haplotypes was tested using score tests accounting for missing phase information and missing genotypes at a subset of markers using the haplo.score R function (Schaid, D. J., et al., Am J Hum Genet, 2002. 70(2): p. 425-34). ORs attached to haplotypes were estimated under the generalized linear model with missing phase information of Lake et al. (Lake, S. L., et al., Hum Hered, 2003. 55(1): p. 56-65) using the haplo.cc R function.

Association Analysis in the Family Sample.

Allelic and genotypic log-odds ratios (ORs) were estimated and Wald tests of association were performed under a logistic model estimated using generalized estimating equations (GEEs) with an independence working correlation structure between the subjects in the same family and an empirical variance estimate robust to intra-familial correlation (Zeger, S. L., K. Y. Liang, and P. S. Albert, Biometrics, 1988. 44(4): p. 1049-6). The same approach was then applied to the combined case-control and family samples. The generalized disequilibrium test (GDT) (Chen, W. M., A. Manichaikul, and S. S. Rich, Am J Hum Genet, 2009. 85(3): p. 364-76), a score test robust to population stratification, was also applied to confirm the GEE Wald test results. For haplotypic association, the most likely haplotype pair for each subject given the genotype data on the entire kindred was inferred by maximum likelihood using Superlink (Fishelson, M., N. Dovgolevsky, and D. Geiger, Hum Hered, 2005. 59(1): p. 41-60, cbl-fog.cs.technion.ac.il/superlink/) and haplotypes were recoded as alleles of a multi-allelic marker for the analysis. All statistical tests were two-sided.

Association Analysis in the Combined Case-Control and Family Samples.

The logistic regression model included a term for the allele count of a SNP and a term for the sample of origin (case-control vs. family) to adjust for differences in allele frequency between the samples. The model was estimated using GEE as for the analysis in the family sample, with the subjects from the case-control sample treated as one-member families.

Evaluation of Linkage Disequilibrium (LD).

The squared correlation and the Lewontin's D′ coefficient among pairs of genotyped SNPs were estimated in the control sample using standard algorithms implemented in the R function LD.

Age of Onset.

First, the mean age of onset between genotypes in patients was compared. Second, genetic association in the kindred sample was tested by comparing the patients with onset before age 26 to the NAARs. Age 26 was chosen based on the median age of first definitive episode of SZ in the kindred sample. In the case-control sample the SZ patients with onset before age 26 were compared to the controls.

Genotyping of the Deletions

A semi-automated genotyping procedure using laser infrared automatic LiCor™ DNA sequencers (Li-Cor, Lincoln, Nebr., USA) was used. PCR primers were synthesized (IDT, Coralville, USA) after adding an M13 tail to the forward primer. PCR fragments were amplified in presence of an infrared tagged M13 primer and separated according to their size in 6% acrylamide-urea gels run on the sequencers. Ins/Del genotypes were called automatically from electronic gel image using SAGA™ software (LICOR). After automatic genotyping manual editing of the results was performed if needed. Genotyping was made blind to the phenotypes. Mendelian inheritance was checked using PedCheck™ software (O'Connell, J. R. and D. E. Weeks, American Journal of Human Genetics, 1998. 63: p. 259-266). Subjects who failed the Mendelian test were reanalyzed completely, that is, from the PCR to the genotyping. When Mendelian errors persisted, these genotypes were discarded. Missing genotypes plus those discarded reached less than 1% of the total sample for all three Ins/Del markers. Some 10% of blind replicates were also included randomly among the DNAs for genotyping quality control: the replication rate was set at 95% that was reach by all three Ins/Del markers.

### Example 2

### Matching of Cases to Controls on Ancestry

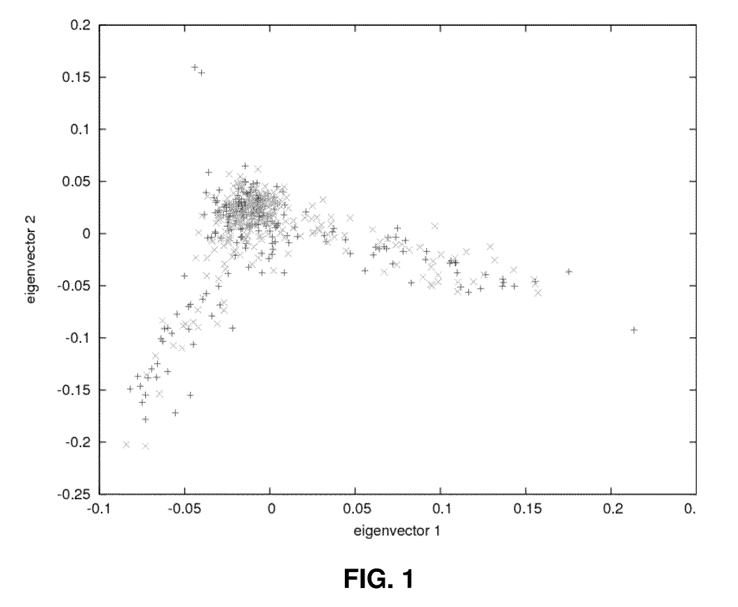

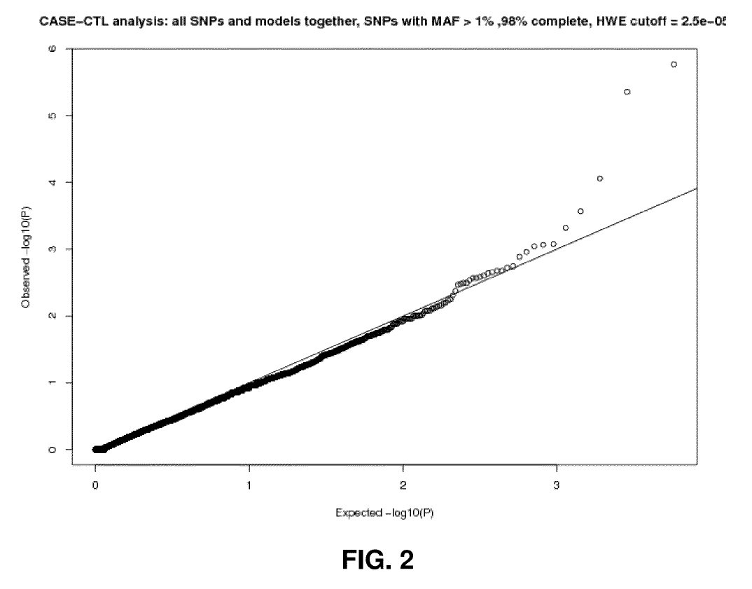

Comparison of the case and control groups along the first ten PCs revealed no important difference (p-values between 0.033 and 0.72). FIG. 1 shows the near perfect overlap between the two groups on the first two PCs. The inflation factor estimated by the genomic control method applied to the genotype data was only 1.006. The good fit of the observed distribution of p-values to the expected one in the candidate region can be seen on a quantile-quantile plot (FIG. 2). In consideration of this absence of evidence of population substructure differences between cases and controls, it was decided to not apply any correction.

### Example 3

### Association with Genotyped SNPs in the Case-Control Sample

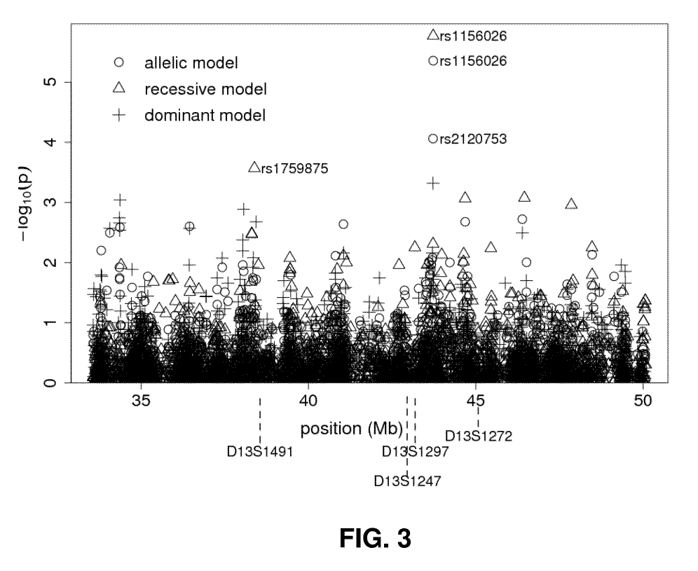

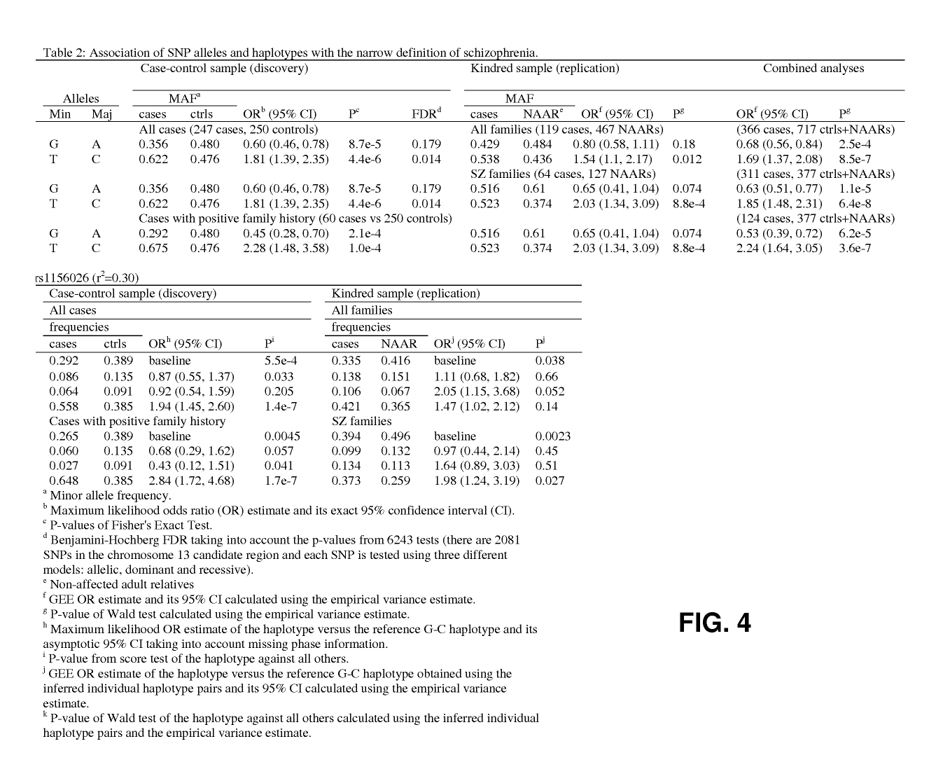

The SNP rs1156026 was the only SNP associated to SZ with a FDR<0.05 in the primary analysis of the SNPs individually using Fisher's exact test, with an OR of 1.81 for the T allele (FIGS. 3 and 4) and 2.63 (95% confidence interval (CI) [1.74, 4.00]) for the TT genotype against the others. Results with the Cochran-Armitage trend test were nearly identical to the Fisher exact allelic test. When the subset of 60 cases with positive family history of SZ, psychosis or paranoia in first, second or third degree relatives was compared to the control group, rs1156026 remained the SNP with the lowest p-value in the region. The SNP effect size was larger in this familial subset (T allele OR=2.28, TT genotype OR=3.10, 95% CI [1.64, 5.86]) but the greatly reduced number of cases resulted in a less significant association (Table 2, FIG. 4). When the case sample was restricted to the 133 cases with onset of SZ before age 26 rs1156026 was again the SNP with the lowest p-value in the region. The ORs remained about the same as in the primary analysis of the full case group and the p-value was less significant because of the lower power provided by a smaller sample. There was no significant difference in mean age of onset between the three rs1156026 genotypes. SNP rs1156026 is located about 500 kb from D13S1297, the marker where the linkage signal peaked in the kindred sample. The sequence of SNP rs1156026 is:

CATAAGGACACTTTGAGGAAGACCCA[C/T]TTCCTGCAGGCAAGCAGGATGAAG T (SEQ ID NO: 1).

### Example 4

### Association with Untyped Variants

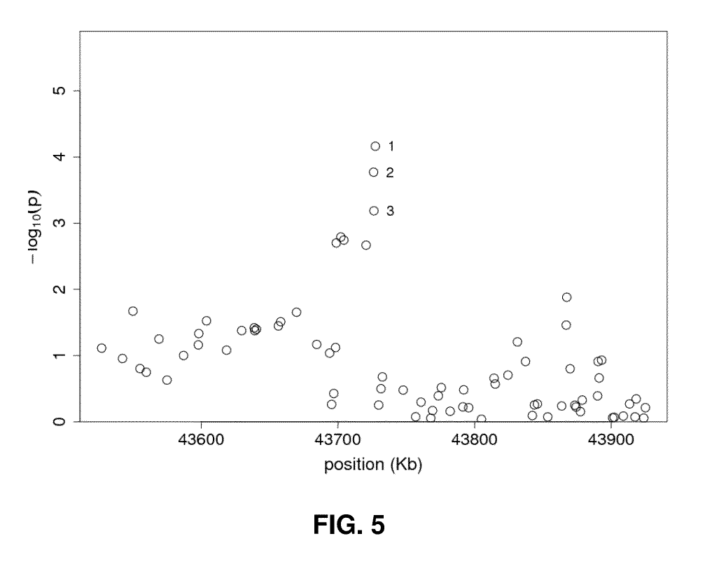

Imputation of SNPs located within 200 kb of rs1156026 using the 1000 Genome Project data revealed no other SNP with stronger association. The lowest p-value obtained was 2.9×10⁻⁵, with rs2657116. Over the same interval, the two windows of three consecutive SNPs showing the strongest association to SZ in global haplotype association tests included rs1156026 (FIG. 5). Windows of five consecutive SNPs yielded no p-value below 10⁻⁴. However, the examination of the association of individual haplotypes in the top two three-SNP windows revealed that the rs2120753-rs2657100-rs1156026 AAT haplotype was associated to SZ with a p-value of 2×10⁻⁵ after correction for testing eight haplotypes, and that the AGT haplotype was also associated to SZ. This indicated that the association was driven by the rs2120753-rs1156026 AT haplotype. It was therefore tested association with haplotypes formed by these two correlated SNPs and obtained an odds ratio of 1.94 for the AT haplotype compared to the reference GC haplotype (Table 2, FIG. 4). When the subset of 60 cases with positive family history was compared to the control group, the odds ratio for the AT haplotype raised to 2.84. In both instances, the uncorrected p-value of the score test of the AT haplotype versus all others is close to 10⁻⁷.

### Example 5

### Conditional Analysis

The association of the genotyped and imputed SNPs conditional on the T/T genotype and the allele count of rs1156026 was tested to determine whether other SNPs are associated to SZ independently of rs1156026. All p-values were non-significant considering multiple testing (p>0.0005). The same conclusion was obtained when conditioning on the allele count at rs2120753 (p>0.001), but it is noted that rs1156026 ranked in first place.

### Example 6

### Linkage Disequilibrium (LD)

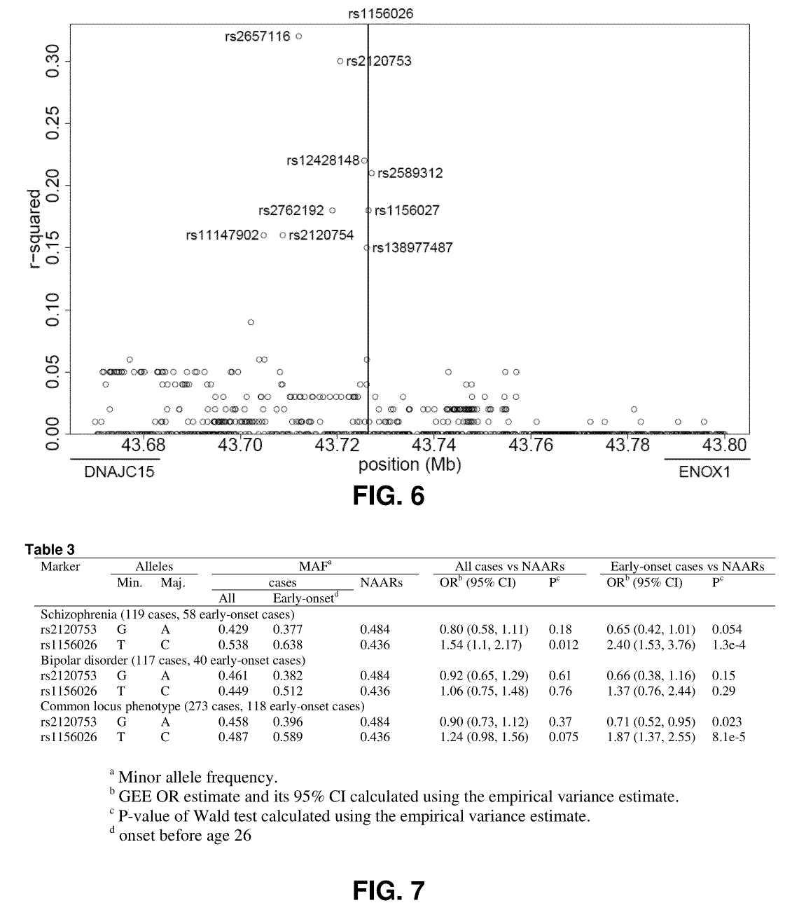

The LD between rs1156026 and other neighboring frequent SNPs was examined in subjects of European ancestry in the 1000 Genomes Project to interpret the association results in the present dataset and other studies. Only 8 SNPs and one insertion/deletion (indel) are correlated at a r²>0.1 with rs1156026 and all are within 25 kb (FIG. 6). Among them, only rs2120753 was genotyped, and the r² with rs1156026 in the control sample is similar to the 1000 Genomes estimate.

### Example 7

### Copy Number Variants (CNVs)

CNVs were detected in 245 cases and 137 controls genotyped in the same batch, to ensure that genotyping signal intensities were comparable. The region from rs1998697 to rs9574453 spanning 6 kb is deleted in one case and duplicated in another. A deletion spanning 10 kb from rs1407608 to rs9646096 has been detected in a control. Overlapping duplications and deletions have been reported in the Database of Genomic Variants (DGV) (Zhang, J., et al., Cytogenet Genome Res, 2006. 115(3-4): p. 205-14, projects.tcag.ca/variation). A duplication spanning 9 kb from rs11619167 to rs2234211 detected in a case has not been reported in the DGV.

### Example 8

### Replication of the SNP Association in the Kindred Sample

A significant association of the T allele of SNP rs1156026 to SZ was also detected in the kindred sample. FIG. 4 shows the results of the GEE analysis for the narrow SZ phenotype (results for the broad SZ phenotype were similar). The estimated OR of 1.54 increased to 2.03 when the analysis was restricted to the sample of families where SZ was the predominant disorder, and the statistical significance of the GEE Wald test also improved from 0.012 to 8.8×10⁻⁴. The GDT gave a similar result in the full sample (p=0.027) but a less significant one in the subsample of SZ families (p=0.072). The combination of the case-control sample with the sub-sample of SZ families gave the strongest overall evidence of association (p=6.4×10⁻⁸). Contrary to the case-control sample, little association was observed with the rs2120753 A allele, and the rs2120753-rs1156026 AT haplotype was no more strongly associated to SZ than the T allele of SNP rs1156026 by itself. Instead, the GC haplotype appeared protective against SZ. Also, there was less evidence of association under the recessive model in the kindred sample than in the case-control sample. The association to the narrow BP and CL phenotype was also examined, which gave linkage signals in the kindred sample (Maziade, M., et al., Eur J Hum Genet, 2009. 17(8): p. 1034-42). Neither rs2120753 nor rs1156026 were significantly associated (Table 3, FIG. 7).

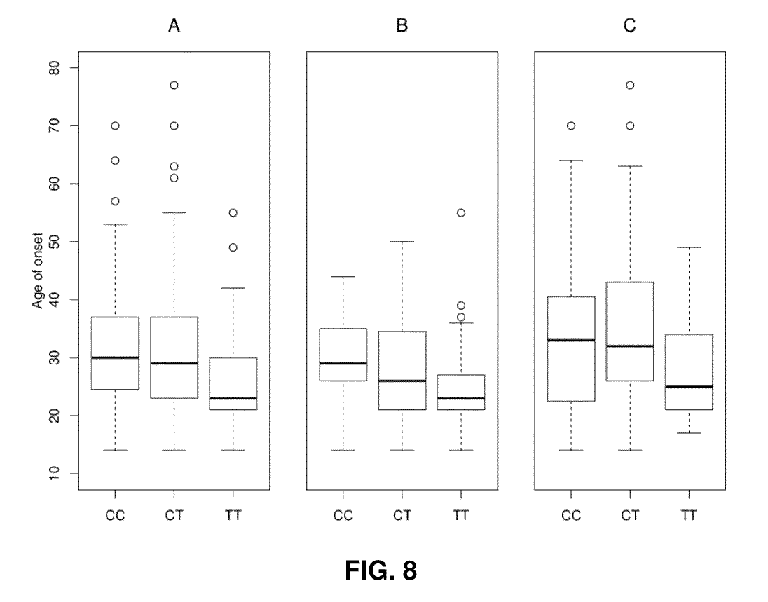

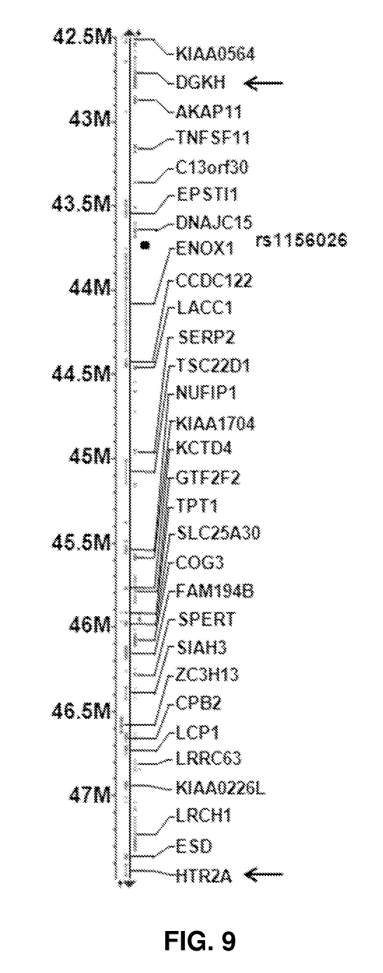

In the kindred sample the mean age at the first probable episode was 26.2 years (standard deviation (SD)=7.8) for narrow SZ, which is close to reports in general samples of SZ (Hafner H, Maurer K, Loffler W, Riecher-Rossler A, Br J Psychiatry, 1993. 162:80-86. Kirkbride J B, Errazuriz A, Croudace T J, Morgan C, Jackson D, Boydell J, et al. PLoS One, 2012. 7:e31660. Rajji T K, Ismail Z, Mulsant B H, Br J Psychiatry, 2009. 195:286-293). For narrow BP the mean onset age was 30.8 years (SD=11.8). Age of onset depended on rs1156026 genotype for the narrow BP (p=0.017) and CL (p=1.1×10⁻⁴) phenotypes in 2 df F tests comparing the three genotypes, driven mainly by an earlier onset in TT carriers (FIG. 8, panels a and b). For SZ, a linear decreasing trend in the mean age of onset with the number of T alleles was observed (−2 years per copy of the T allele, p=0.034, FIG. 8, panel c). The age of onset in carriers of the TT genotype was on average approximately 5 years earlier than in carriers of the CC genotype for both SZ and BP, and therefore also CL. The case sample was then restricted to those with onset of their disorder before age 26 in the association analysis (excluding older onset cases from the analysis). For SZ, the OR for the T allele of rs11556026 increased to 2.40 (Table 3, FIG. 7). The enrichment in T alleles in BP with onset before age 26 was insufficient to obtain a significant association. Grouping together SZ, BP and SAD cases with onset before age 26 in the CL phenotype however revealed a significant association with the T allele (p=8.1×10⁻⁵), with an OR=1.87. Analysis of the haplotypes formed by rs2120753 and rs1156026 did not improve the signal in any of the above analyses, the association being driven by the rs1156026 T allele.

### Example 9

### Linkage to the Associated SNPs

Parametric linkage analysis using the same model parameters as in previous reports (Maziade, M., et al., Molecular Psychiatry, 2005. 10(5): p. 486-99; Maziade, M., et al., Eur J Hum Genet, 2009. 17(8): p. 1034-42) revealed no evidence of linkage to rs2120753 and rs1156026 either analyzed individually or together in a multipoint analysis. The maximized logarithm of the odds (MOD) score was equal to 0.6 for the broad CL phenotype and 0.21 for the broad SZ phenotype.

### Example 10

### Prediction of Schizophrenia by Combining Measures of Episodic Memory with the SNP Rs1156026 T Allele

Young high-risk (HR) offspring of schizophrenia (SZ) or bipolar disorder (BP) parents share important deficits in both verbal episodic memory (VEM) and visual episodic memory (VisEM) (Maziade, M., et al., Schizophr Bull, 2009. 35(5): p. 919-30), in fact the largest differences with normal controls were for these 2 cognitive domains (effect sizes (ESs) of 0.8-0.9). It was assessed in the studies herein whether the combined use of VEM or VisEM impairments and the SNP rs1156026 T allele identified above may be used in the prediction of SZ.

Methods

Descriptions of samples and neuropsychological assessments are as described in Maziade et al., 2010 (Maziade, M., et al., Schizophr Bull, 2010. 37(6): p. 1218-28).

Nonaffected Adult Relatives (NAARs) Subsample.

The inclusion criteria were (1) having a first-degree relative with a definite DSM-IV SZ or BP disorder and (2) having had a neuropsychological evaluation before being 55-year old.

Patients Subsample.

The adult members affected by SZ came from the same large densely affected multigenerational kindreds and from the sample of unrelated SZ cases. The inclusion criteria were (1) a definite DSM-IV SZ or a BP diagnosis, (2) having undergone a neuropsychological evaluation before age 55, and (3) being in a clinical status allowing a reliable cognitive assessment. The exclusion criteria were a brain disorder, trauma, and metabolic disorder known to cause neuropsychological impairments.

Neuropsychological Assessments.

In this study, we focused on the free recall measures of tests because they showed the largest ESs in the comparison of offspring with controls. VEM was assessed with the California Verbal Learning Test (CVLT) (Delis, D. C., et al., California Verbal Learning Test Manual, 1987, San Antonio, Tex.: Psychological Corporation) “total and delayed recalls” in which subjects had to learn a series of words presented orally over 5 trials, and to immediately recall them after each presentation (total recall of 5 trials), or with a 20-min delay (delayed recall). VisEM was assessed with the Rey Complex Figure Test (RCF) (Meyers, J. E. and K. R. Meyers, Rey Complex Figure Test and Recognition Trial (RCFT) 1995, Odessa, Fla.: Psychological Assessment Resources) immediate and delayed recalls. Subjects had to copy a figure and then recall it after 3 min (immediate recall) and 30 min (delayed recall). The tests were administered in the same order in all subjects. Subjects were individually assessed in a quiet room for a period of 3-4 h by certified psychologists or Ph.D. students who were blind to diagnoses and supervised by a senior neuropsychologist (N.R.). Pauses were offered when needed.

Statistical Analysis.

VEM and VisEM test results were converted into percentiles based on age and gender specific published scales. An impairment on VEM or VisEM was defined as a test result below the 16^{th} percentile. Logistic regression was used to model the association of VEM or VisEM impairment and the number of rs1156026 T alleles with SZ diagnosis, separately in univariate analyses and in bivariate analyses of one memory impairment with the SNP (models with more than two variables were not fitted due to sample size constraints). In the bivariate models, the interaction term between the memory impairment and the SNP was tested. Interaction terms that were not significant at the 0.05 level were removed from the model, leaving only the independent main effect terms. Odds ratios (ORs) between two combinations of levels of the variables estimated from the logistic models approximate the relative risk of SZ between the two combinations due to the low prevalence of SZ.

The predictive power of the logistic models was measured by the area under the receiver operating curve (ROC) produced by the model. The ROC curve shows the sensitivity of the model SZ prediction against its false positive rate (one minus the specificity), for all thresholds of the predicted probability of SZ above which a prediction of SZ would be issued. The area under the ROC curve varies from 0.5 (no predictive power) to 1 (perfect prediction).

Results

A total of 52 SZ cases and 93 NAARs underwent a neuropsychological assessment and were genotyped for the SNP rs1156026. The interaction term was not statistically significant at the 0.05 level in any of the memory impairment—SNP combinations tested. All bivariate models therefore contain only main effect terms for the rs1156026 T allele and the memory impairments were similar in the univariate and bivariate analyses, indicating that the two factors were independently associated to SZ (Tables 4 and 5). Logistic models without interaction terms imply that ORs for two variables in the same model are multiplicative. This translates for instance in a risk of SZ 17.4 times greater in subjects with a VisEM impairment (immediate recall) and a CT genotype compared to no impairment and a CC genotype (Table 4) and a risk of SZ 18.1 times greater in subjects with a VEM impairment (total recall) compared to no impairment and a CC genotype (Table 5).

TABLE 4
Logistic regression modeling of schizophrenia (SZ) diagnosis
as a function of the number of SNP rs1156026 T alleles and VisEM
deficits (either delayed recall or immediate recall) in a sample
of 51 SZ cases and 91 unaffected adult relatives.
VisEM: immediate recall
Joint analysis model with variables rs1156026
and VisEM: p-value of LR test = 3.1e−06

| rs1156026 | VisEM | OR (95% CI) |
|---|---|---|
| CC | No deficit | 1.00 (NA) |
| CT | No deficit | 2.17 (1.12, 4.19) |
| CC | deficit | 8.04 (2.77, 23.36) |
| CT | deficit | 17.43 (4.91, 61.82) |

Models for marginal effects

| variable rs1156026 only | | Variable VisEM only | |
|---|---|---|---|
| p-value of LR | | p-value of LR | |
| test | OR (95% CI) | test | OR (95% CI) |
| 0.0064 | 2.39 (1.26, 4.54) | 4.2e−06 | 8.71 (3.06, 24.76) |

VisEM: delayed recall
Joint analysis model with variables rs1156026
and VisEM: p-value of LR test = 0.00029

| rs1156026 | VisEM | OR (95% CI) |
|---|---|---|
| CC | No deficit | 1.00 (NA) |
| CT | No deficit | 2.28 (1.18, 4.40) |
| CC | deficit | 4.06 (1.59, 10.38) |
| CT | deficit | 9.25 (2.89, 29.57) |

Models for marginal effects

| variable rs1156026 only | | Variable VisEM only | |
|---|---|---|---|
| p-value of | | p-value of | |
| LR test | OR (95% CI) | LR test | OR (95% CI) |
| 0.0064 | 2.39 (1.26, 4.54) | 0.00092 | 4.36 (1.75, 10.85) |

TABLE 5
Logistic regression modeling of schizophrenia (SZ) diagnosis
as a function of the number of SNP rs1156026 T alleles and
VEM deficits (either delayed recall or total recall) in a
sample of 51 SZ cases and 92 unaffected adult relatives.
VEM: total recall
Joint analysis model with variables rs1156026
and VEM: p-value of LR test = 3.2e−5

| rs1156026 | VEM | OR (95% CI) |
|---|---|---|
| CC | No deficit | 1.00 (NA) |
| CT | No deficit | 2.12 (1.08, 4.19) |
| CC | deficit | 8.53 (2.58, 28.25) |
| CT | deficit | 18.11 (4.57, 71.8) |

Models for marginal effects

| | |
|---|---|
| variable rs1156026 only | Variable VEM only |

| p-value of | | p-value of | |
|---|---|---|---|
| LR test | OR (95% CI) | LR test | OR (95% CI) |
| 0.0064 | 2.39 (1.26, 4.54) | 9.9e−5 | 8.79 (2.79, 27.73) |

VEM: delayed recall
Joint analysis model with variables rs1156026
and VEM: p-value of LR test = 5.3e−5

| rs1156026 | VEM | OR (95% CI) |
|---|---|---|
| CC | No deficit | 1.00 (NA) |
| CT | No deficit | 2.51 (1.28, 4.96) |
| CC | deficit | 5.26 (2.04, 13.62) |
| CT | deficit | 13.24 (3.82, 45.92) |

Models for marginal effects

| | |
|---|---|
| variable rs1156026 only | Variable VEM only |

| p-value of | | p-value of | |
|---|---|---|---|
| LR test | OR (95% CI) | LR test | OR (95% CI) |
| 0.0064 | 2.39 (1.26, 4.54) | 0.00083 | 4.45 (1.83, 10.85) |

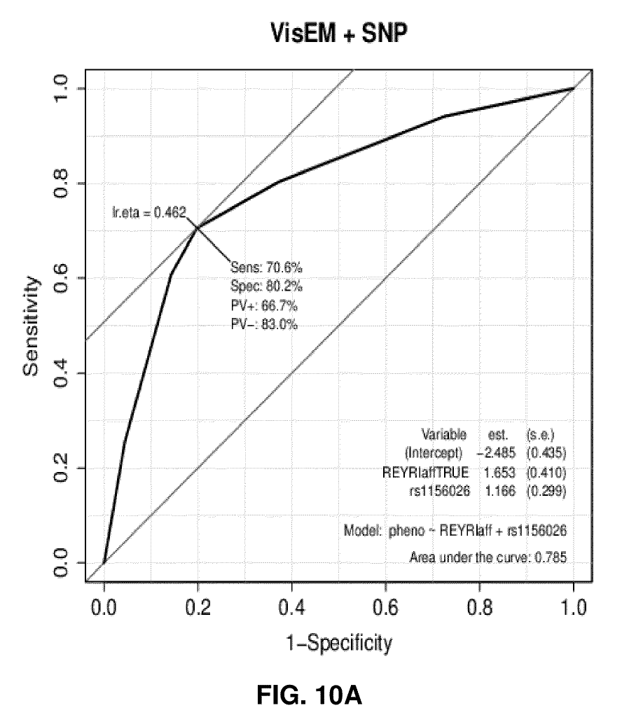

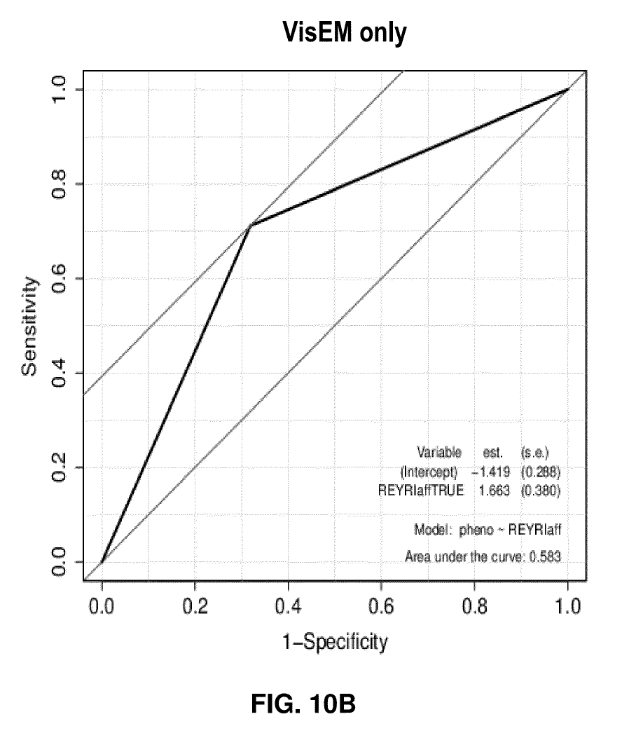

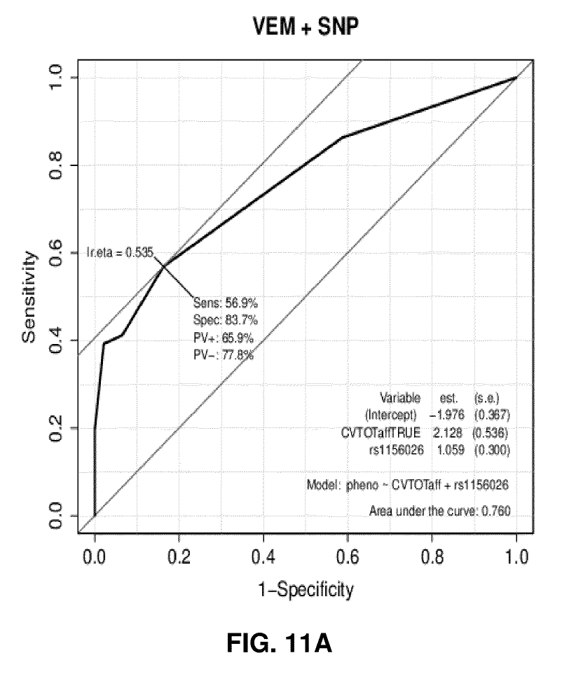

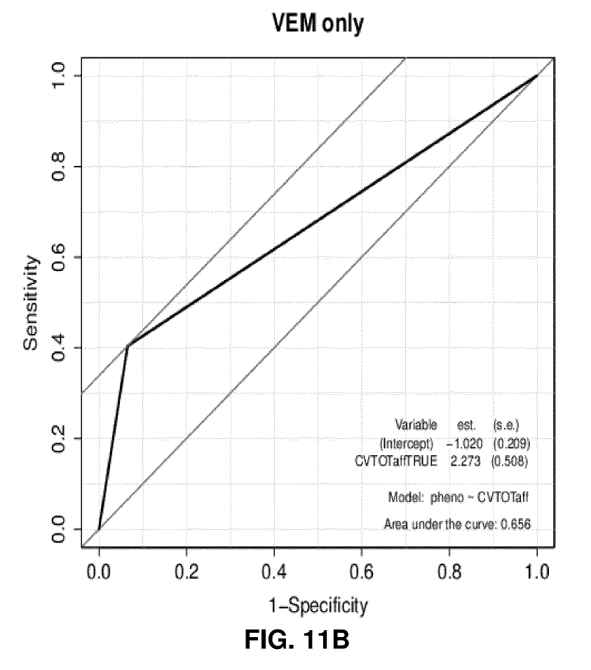

The ROC curve for the bivariate models and the model with the memory impairment only are displayed on FIGS. 10A and 10B for VisEM immediate recall and on FIGS. 11A and 11B for VEM total recall. For VisEM, the area under the ROC curve improves from 0.58 to 0.79 when adding the T allele of rs1156026 to the logistic model. For VEM total recall, the corresponding improvement is from 0.66 to 0.76. At the point of highest discrimination, a diagnostic test involving VisEM immediate recall and the SNP rs1156026 would have a sensitivity of 70.6% and a specificity of 80.2%. For VEM total recall and the SNP rs1156026, the corresponding figures are a sensitivity of 56.9% and a specificity of 83.7%.

### Example 11

### Association of SNPs Genotyped at 13q13-14 and Tourette Syndrome

Association analyses were performed on a sample of 91 nuclear families (81 trios, 8 dyads and 2 families) in which 93 subjects are affected by Tourette's syndrome (TS). Likelihood ratio tests of association and odds ratio (OR) estimates were obtained for SNP alleles and haplotypes of selected SNPs by maximizing the likelihood function of Dudbridge (Hum Hered 2008; 66: 87-98) using the Unphased package (http://unphased.sourceforge.net/), which handles partially missing parental genotypes and multiple siblings. Linkage disequilibrium (LD) coefficients between SNPs were estimated from parental haplotypes using the Family Based Association Testing (FBAT) software package.

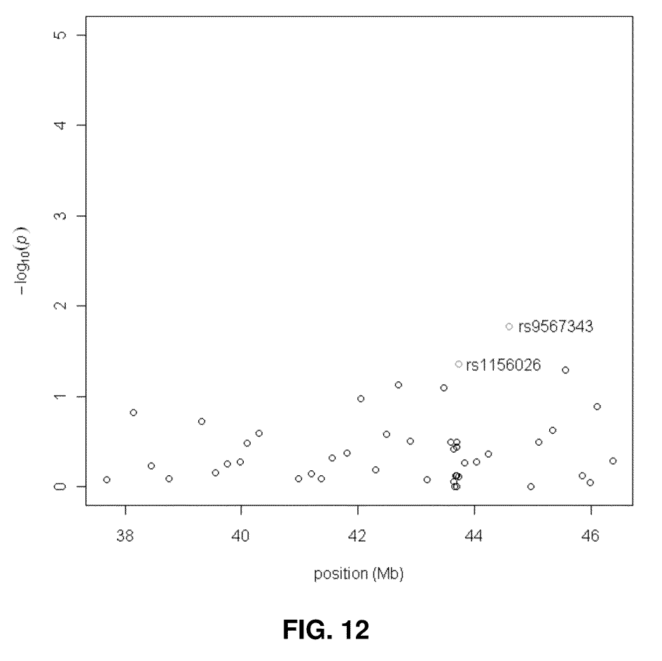

FIG. 12 shows the results of the association test with the 45 SNPs. Association to TS at the 0.05 level was detected with the C allele of rs1156026 (OR=1.6, p=0.043) and the G allele of rs9567343 (OR=1.9, p=0.016). There was a moderate level of LD between these two SNPs (D′=0.26, r²=0.009). These two SNPs were selected with rs1323142 and rs883877, the two SNPs in between showing moderate LD (r²<0.03) with either rs1156026 or rs9567343 to evaluate haplotypic association. It was observed that TS cases are more likely to receive any haplotype other than the T-G-C-A haplotype from SNPs rs1156026-rs1323142-rs883877-rs9567343 with an OR=3.2 (for all other haplotypes against T-G-C-A, p=0.004), implying TGCA as being a protective haplotype against TS (see Table 6).

TABLE 6
Association of the most frequent haplotypes
(freq > 0.5) of the SNPs rs1156026-rs1323142-
rs883877-rs9567343 with Tourette syndrome.

| Haplotype | Frequency | T-Freq | U-Freq | OR (95% CI)^{a} | p-value^{b} |
|---|---|---|---|---|---|
| T-A-C-A | .210 | .185 | .229 | 0.70 (0.41, 1.21) | 0.257 |
| C-A-C-A | .174 | .212 | .143 | 1.74 (0.94, 3.20) | 0.084 |
| T-G-T-A | .134 | .093 | .143 | 0.57 (0.26, 1.22) | 0.140 |
| T-G-C-A | .098 | .052 | .165 | 0.32 (0.14, 0.73) | 0.004 |
| C-G-C-A | .067 | .082 | .054 | 1.57 (0.59, 4.23) | 0.402 |
| C-G-T-A | .059 | .064 | .061 | 1.04 (0.42, 2.60) | 0.934 |

^{a}Maximum likelihood estimate of the OR for the listed haplotype versus all others and its asymptotic 95% CI
^{b}P-value from score test of the haplotype versus all other haplotypes

### Example 12

### Association with Deletions and the SNP Rs1156026 in the Sample of Kindreds Linked to the 13q13-q14 Region

The three deletions rs10569822, rs113886253 and rs146020684 were found on a haplotype segregating with the CL narrow phenotype in kindreds linked to the 13q13-q14 region. These deletions located in introns of the DnaJ (Hsp40) homolog, subfamily C, member 15 (DNAJC15) gene were genotyped in our kindred sample. Kindreds linked to the 13q13-q14 region were defined as the 10 kindreds with a nonparametric linkage (NPL) score >1 for the CL narrow phenotype (Maziade, M., et al., Eur J Hum Genet, 2009. 17(8): p. 1034-42) and allelic association of the three deletions and the SNP rs1156026 defined herein to the SZ, BP and CL narrow phenotypes in this subset of linked kindreds were examined (Table 7).

TABLE 7
Association of deletions and SNP alleles for schizophrenia, bipolar
disorder and the common locus phenotype in the linked kindred sample

| | | | | | | |
|---|---|---|---|---|---|---|
| | Minor | Major | Case | NAAR^{b} | | |
| Marker | allele | allele | MAF^{a} | MAF | OR^{c} (95% CI) | P^{d} |

Bipolar disorder, narrow

| | | | | | | |
|---|---|---|---|---|---|---|
| rs10569822 | 172 | 174 | 0.304 | 0.196 | 1.79 (1.03, 3.11) | 0.038 |
| rs113886253 | 211 | 217 | 0.304 | 0.201 | 1.71 (0.95, 3.08) | 0.075 |
| rs146020684 | 234 | Other | 0.304 | 0.203 | 1.7 (0.94, 3.05) | 0.078 |
| | | alleles | | | | |
| rs1156026 | T | C | 0.404 | 0.348 | 1.27 (0.54, 2.97) | 0.582 |

Schizophrenia, narrow

| | | | | | | |
|---|---|---|---|---|---|---|
| rs10569822 | 172 | 174 | 0.233 | 0.196 | 1.25 (0.49, 3.19) | 0.638 |
| rs113886253 | 211 | 217 | 0.233 | 0.201 | 1.2 (0.51, 2.83) | 0.672 |
| rs146020684 | 234 | Other | 0.224 | 0.203 | 1.13 (0.49, 2.64) | 0.773 |
| | | alleles | | | | |
| rs1156026 | T | C | 0.533 | 0.348 | 2.31 (1.42, 3.76) | 0.0008 |

Common locus phenotype, narrow

| | | | | | | |
|---|---|---|---|---|---|---|
| rs10569822 | 172 | 174 | 0.243 | 0.196 | 1.31 (0.84, 2.05) | 0.231 |
| rs113886253 | 211 | 217 | 0.243 | 0.201 | 1.26 (0.83, 1.93) | 0.280 |
| rs146020684 | 234 | Other | 0.239 | 0.203 | 1.23 (0.8, 1.88) | 0.346 |
| | | alleles | | | | |
| rs1156026 | T | C | 0.456 | 0.348 | 1.57 (1, 2.45) | 0.048 |

^{a}MAF: minor allele frequency
^{b}NAAR: non affected adult relative
^{c}OR: odds ratio of the phenotype versus the NAARs, CI: confidence interval
^{d}P: two-sided allelic Wald test p-value

The three deletions were significantly associated to BP at the 0.1 level (0.05 level for rs10569822), but not to SZ or the CL phenotype combining SZ, BP and SAD. The association of the T allele of rs1156026 to SZ in these kindreds linked to the 13q13-q14 region is stronger than the association in the full kindred sample reported in Example 8 above. However, no association of rs1156026 to BP was observed.

### Example 13

### DNAJC15 Deletions Interacting with SNP Rs1156026 in Predicting Schizophrenia and Bipolar Disorder

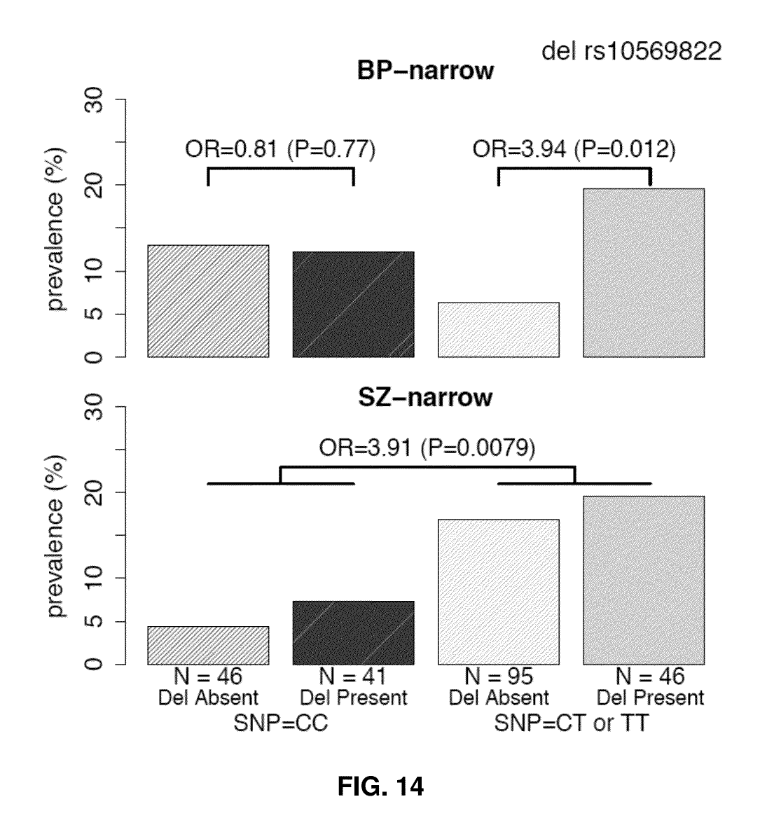

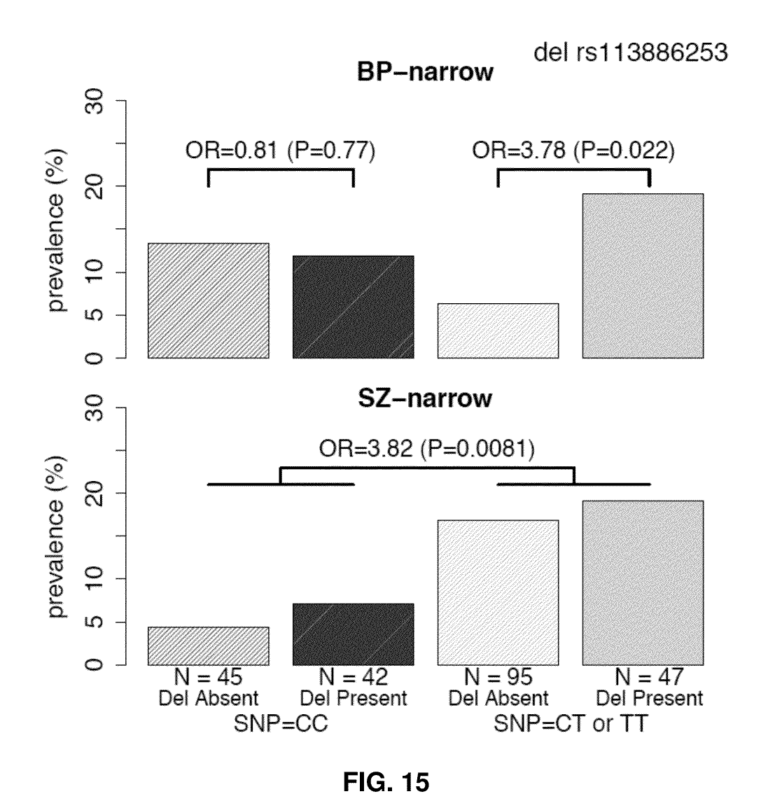

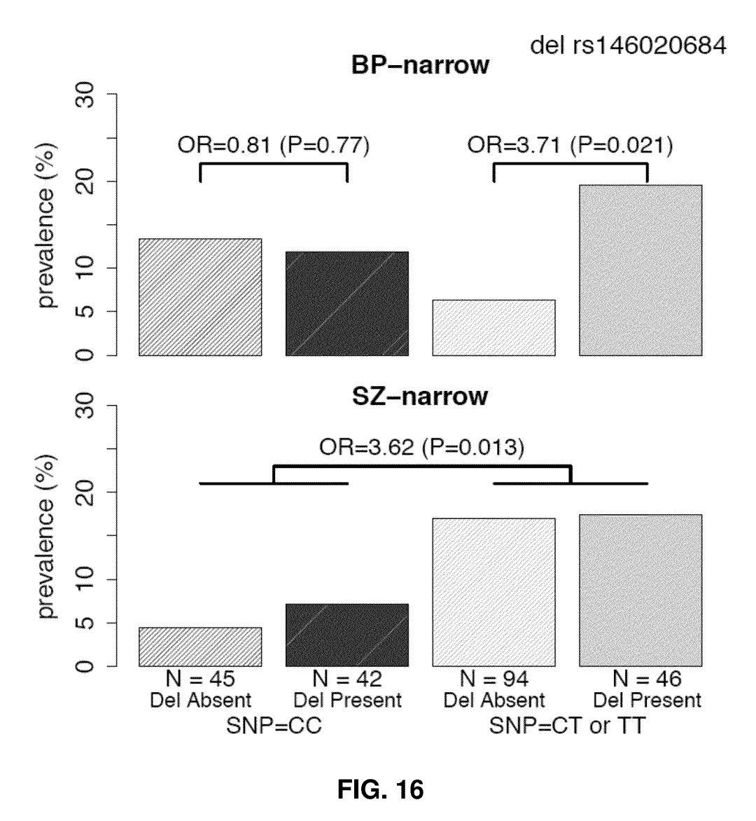

The three DNAJC15 intronic deletions rs10569822, rs113886253 and rs146020684 are strongly correlated (r²>0.9 in unaffected members of the kindred sample). Since the three deletions are essentially interchangeable in the association results, they will be collectively referred to as the deletions. The association of the deletions and rs1156026 jointly was next examined using a dominant coding given the low frequency of homozygous carriers of the deletion. FIGS. 14-16 show that the deletions are associated to BP among carriers of the T allele of SNP rs1156026 (FIGS. 14-16, top) in the linked kindreds. This association was not observed in subjects with the CC genotype of SNP rs1156026. This implies a qualitative interaction between the two genetic variants in their association to BP, and results in an interaction under a logistic model of BP for rs10569822 (p=0.09), rs113886253 (p=0.11) and rs146020684 (p=0.11).

The odds ratio of SZ versus NAARs between carriers and non-carriers of the T allele of rs1156026 was shown to be independent of the deletions. Consistent with the results in Table 7, the deletions do not show any significant evidence of association to SZ (FIGS. 14-16, bottom).

Based on these results, a differential diagnosis of SZ and BP in patients presenting a first episode of non-specific psychosis may be achieved via assessment of various combinations of the SNP rs1156026 and one or more of the deletions described herein. For such patients, the above observations on the associations with rs1156026 and the deletions lead to the inferences presented in Table 8.

TABLE 8
Differential diagnosis of patients presenting
a first episode of non-specific psychosis.

| rs1156026 genotype | Deletion carrier | Diagnosis |
|---|---|---|
| CC | No | BP |
| CC | Yes | Unresolved* |
| CT ou TT | No | SZ |
| CT ou TT | Yes | Unresolved* |

*Unresolved means that the SZ/BP ratio is not statistically different from 1.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

1. A method for diagnosing a psychiatric disorder or a predisposition thereto in a subject, said method comprising determining the identity of single nucleotide polymorphism (SNP) rs1156026 set forth in SEQ ID NO: 1 in a biological sample comprising a nucleic acid from said subject, and diagnosing said psychiatric disorder or predisposition thereto based on said identity.

2. The method of claim 1, wherein the identity of SNP rs1156026 is determined by sequencing a region encompassing SNP rs1156026 in the nucleic acid present in said biological sample.

3. (canceled)

4. The method of claim 1, wherein said psychiatric disorder is major psychosis.

5. The method of claim 4, wherein said psychiatric disorder is schizophrenia (SZ).

6. (canceled)

7. The method of claim 4, wherein the presence of one or two thymine (T)-containing alleles of SNP rs1156026 is indicative that said subject suffers from said psychiatric disorder, or has a predisposition thereto.

8. (canceled)

9. The method of claim 7, wherein the presence of two thymine (T)-containing alleles of SNP rs1156026 is indicative that said subject has a predisposition to develop said psychiatric disorder before the age of about 26.

10. The method of claim 1, further comprising measuring an episodic memory parameter in said subject.

11. The method of claim 10, wherein said episodic memory parameter is verbal episodic memory (VEM), visual episodic memory (VisEM), or both.

12. The method of claim 1, wherein said psychiatric disorder is Tourette's syndrome (TS).

13. The method of claim 12, wherein the presence of one or two cytosine (C)-containing alleles of SNP rs1156026 is indicative that said subject suffers from TS, or has a predisposition thereto.

14. The method of claim 12, wherein said method further comprises determining the identity of one or more of SNPs rs1323142 (SEQ ID NO: 6), rs883877 (SEQ ID NO: 7) and rs9567343 (SEQ ID NO: 8) in a biological sample from said subject.

15. A kit for diagnosing a psychiatric disorder or a predisposition thereto in a subject, or for determining the risk that a subject develops major psychosis before the age of about 26, the kit comprising (i) at least one reagent, wherein said at least one reagent is for determining the identity of single nucleotide polymorphism (SNP) rs1156026 set forth in SEQ ID NO:1 in a biological sample from the subject, and (ii) a container.

16. (canceled)

17. The kit of claim 15, wherein the identity of SNP rs1156026 is determined by sequencing a region encompassing SNP rs1156026 in a nucleic acid present in said sample.

18. (canceled)

19. The kit of claim 15, wherein said psychiatric disorder is major psychosis.

20. The kit of claim 19, wherein said psychiatric disorder is schizophrenia (SZ).

21. The kit of claim 15, wherein said psychiatric disorder is Tourette's syndrome (TS).

22. The kit of claim 21, further comprising at least one reagent for determining the identity of one or more of SNPs rs1323142 (SEQ ID NO: 6), rs883877 (SEQ ID NO: 7) and rs9567343 (SEQ ID NO: 8) in a biological sample from said subject.

23. The kit of claim 15, wherein said at least one reagent is one or more oligonucleotides.

24-27. (canceled)

28. A method for preventing or treating a psychiatric disorder, said method comprising identifying a subject suffering from a psychiatric disorder or a predisposition thereto using the method of claim 1; and administering a course of therapy or prophylaxis to said subject.

29. The method of claim 28, wherein the course of therapy or prophylaxis comprises administration of a psychotropic medication.
